(19) United States
(12) Patent Application Publication
(10) Pub. No.: US 2024/0131075 A1
(43) Pub. Date: Apr. 25, 2024

(54) USE OF MBV FOR TREATING AUTOIMMUNE DISEASE

(71) Applicant: University of Pittsburgh - Of the Commonwealth System of Higher Education, Pittsburgh, PA (US)

(72) Inventors: Stephen Badylak, Pittsburgh, PA (US); George S. Hussey, Pittsburgh, PA (US); Raphael Crum, Pittsburgh, PA (US)

(73) Assignee: University of Pittsburgh - Of the Commonwealth System of Higher Education, Pittsburgh, PA (US)

(21) Appl. No.: 17/769,592

(22) PCT Filed: Oct. 22, 2020
(86) PCT No.: PCT/US2020/056899

§ 371 Date: Apr. 14, 2022

Related U.S. Application Data
(60) Provisional application No. 62/925,129, filed on Oct. 23, 2019.

(51) Int. Cl. A61K 35/22; A61K 9/127; A61K 35/38; A61P 17/06; A61P 19/02; A61P 29/00

(52) U.S. Cl. CPC A61K 35/22; A61K 9/127; A61K 35/38; A61P 17/06; A61P 19/02; A61P 29/00

(57) ABSTRACT

Methods are disclosed for treating an autoimmune disorder in a subject in need thereof. These methods include administering to the subject a pharmaceutical preparation comprising isolated matrix bound vesicles (MBV) derived from extracellular matrix. The administration can be systemic. In some embodiments, the subject has rheumatoid arthritis or psoriasis.

### CROSS REFERENCE TO RELATED APPLICATIONS

This claims the benefit of U.S. Application No. 62/925,129, filed Oct. 23, 2019, which is incorporated by reference herein.

### FIELD

This relates to administration of matrix bound vesicles (MBV) for treating autoimmune disorders.

### BACKGROUND

A major challenge in the treatment of autoimmune conditions, such as rheumatoid arthritis, psoriasis, lupus, multiple sclerosis, among others, is to selectively modulate immune responses responsible for autoimmunity while retaining the host protective immune response to infectious agents. To date, the most frequent side effect of currently used immunosuppressive therapies is an increased risk for infections and malignancy. Thus, a need remains for other therapeutic agents for treating these conditions.

### SUMMARY

Methods are disclosed for treating an autoimmune disorder in a subject in need thereof that include administering to the subject a pharmaceutical preparation that includes a therapeutically effective amount of isolated matrix bound nanovesicles (MBV) derived from extracellular matrix, thereby treating the autoimmune disorder. In some non-limiting examples, the administration is systemic. In other non-limiting examples, the disorder is rheumatoid arthritis. In further non-limiting examples, the disorder is psoriasis, lupus, pemphigus, pemphigoid, or multiple sclerosis. The methods can include selecting these subjects for treatment.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1G show morphological characterization of liquid phase extracellular vesicles (EV) and matrix bound nanovesicles (MBV). FIG. 1A shows scanning electron microscopy images of an ECM scaffold derived from urinary bladder matrix (UBM), in which discrete spherical bodies approximately 100 nm in diameter are dispersed throughout the matrix. Scale bars=1 μm. FIG. 1B shows an illustration of the 3T3 fibroblast cell culture model used to selectively harvest vesicles from a liquid-phase (EV) or solid-phase extracellular compartment (MBV). FIG. 1C shows phase contrast microscopy, hematoxylin and eosin (H&E) staining, and 4′,6-diamidino-2-phenylindole (DAPI) staining demonstrating the absence of cells and absence of intact cell nuclei following decellularization. FIG. 1D shows transmission electron microscopy images of liquid-phase EV and MBV isolated from the 3T3 fibroblast cell culture model. Scale bars=100 nm. FIG. 1E shows size distribution plots from nanoparticle tracking analysis (NTA) of liquid-phase EV and MBV isolates from the 3T3 fibroblast cell culture. FIG. 1F shows immunoblot analysis comparing CD9, CD63, CD81 and Hsp70 expression levels in liquid-phase EV and MBV. FIG. 1G shows silver stain analysis of electrophoretically separated proteins in liquid-phase EV and MBV.

FIGS. 2A-2E depict differences in miRNA cargo between EV and MBV. FIG. 2A shows bioanalyzer analysis of total RNA isolated from 3T3 parental cells, their secreted liquid-phase EV, and their MBV. FIG. 2B shows principal-component analysis (PCA) comparing liquid-phase EV (green), MBV (blue) and cellular (red) RNA-seq datasets. FIG. 2C shows volcano plots demonstrating the differential expression of miRNAs in liquid-phase EV, MBV, and the parental cells. The inclusion criteria were a 2-fold difference of log₂ (fold-change) in either direction, with a P-value<0.05. Each dot represents a specific miRNA transcript; green dots to the right of the vertical dashed line (and above the horizontal dashed line) correspond to a relative increase in expression level, and red dots to the left (and above the horizontal dashed line) correspond to a relative decrease in expression level. Blue dots (which appear below the horizontal dashed line) indicate miRNA with no significant change in expression level. FIG. 2D shows RT-qPCR validation of the results of miRNA sequencing, *p<0.05, n=4. FIG. 2E shows Ingenuity Pathway Analysis (IPA functional analysis). Significantly enriched molecular functions identified by IPA functional analysis were determined considering differentially expressed miRNA in MBV (red-bottom bar) and liquid-phase EV (blue-top bar). There is no red bar for cellular growth & proliferation, cell morphology, cell-to-cell signaling, and tissue development; there is no blue bar for digestive system development & function, hepatic system development and function and organ development and function.

FIGS. 3A-3H depict differences in miRNA cargo of MBV based on the cellular origin of the MBV. FIG. 3A shows a phase contrast microscopy image of a decellularized BMSC cell culture plate showing the absence of cells. FIG. 3B shows a transmission electron microscopy image of MBV isolated from the decellularized BMSC culture plate. Scale bars=100 nm. FIG. 3C-FIG. 3E show size distribution plots from nanoparticle tracking analysis (NTA) of MBV isolated from BMSC (FIG. 3C), ASC (FIG. 3D), and UCSC (FIG. 3E) decellularized culture plates. FIG. 3F shows bioanalyzer analysis of total RNA isolated from BMSC, ASC, and UCSC-derived MBV. FIG. 3G shows principal-component analysis (PCA) comparing BMSC MBV (green; middle left), UCSC MBV (blue; top right) and ASC MBV (red; bottom right) RNA-seq datasets. FIG. 3H shows volcano plots demonstrating the differential expression of miRNAs in BMSC, ASC and UCSC-derived MBV. The inclusion criteria was a 2-fold difference of log₂ (fold-change) in either direction with a P-value<0.05. Each dot represents a specific miRNA transcript; green dots to the right of the vertical dashed line (and above the horizontal dashed line) correspond to a relative increase in expression level, and red dots to the left (and above the horizontal dashed line) correspond to a relative decrease in expression level. Blue dots (below the horizontal dashed line) indicate miRNA with no significant change in expression level.

FIGS. 4A-4E show LC/MS characterization of phospholipids between MBV, liquid-phase EV, and the parent cells. FIG. 4A shows a typical total ion chromatogram of phospholipids obtained from MBV. FIG. 4B shows mass spectra of the major phospholipid classes in MBV. Assessment included quantification of saturated (double bond number=0), monounsaturated (double bond number-1) and polyunsaturated (double bond number=2-10) species of phospholipids. FIG. 4C shows pie plots showing the total content of major phospholipids. The data are presented as % of total phospholipids. FIG. 4D and FIG. 4E show the contents of different phospholipid molecular species. The data are presented as heat maps autoscaled to Z-scores and coded blue (low values) to red (high values). Abbreviations are: EV, exosomal vesicles; MBV, matrix-bound vesicles; PC, phosphatidylcholine; PCd, PC diacyl species; PCp, PC plasmalogens; PE, phosphatidylethanolamine, PEd, PE diacyl species; PEp, PE plasmalogens; PI, phosphatidylinositol; PS, phosphatidylserine; BMP, bis-monoacylglycerophosphate; PA, phosphatidic acid; PG, phosphatidylglycerol; and SM, sphingomyelin.

FIGS. 5A-5D show LC/MS characterization and follow-up analyses examining differences in LPE, LPA and LPG between MBV, liquid-phase EV, and the parent cells. FIG. 5A shows typical mass spectra of major lyso-phospholipids obtained from MBV. FIG. 5B shows pie plots showing the total content of major lyso-phospholipids. Data are presented as % of total lyso-phospholipids. FIG. 5C and FIG. 5D show the contents of lyso-phospholipid molecular species. The data are presented as heat maps autoscaled to Z-scores and coded blue (low values) to red (high values), with n=3. Abbreviations are: EV, exosomal vesicles; MBV, matrix-bound vesicles; LPC, lyso-phosphatidylcholine; LPE, lyso-phosphatidylethanolamine; LPI, lyso-phosphatidylinositol; LPS, lyso-phosphatidylserine; LPA, lyso-phosphatidic acid; LPG, lyso-phosphatidylglycerol; and mCL, mono-lyso-cardiolipin.

FIGS. 6A-6C demonstrate that levels of PUFA-containing phospholipids and their oxidatively modified molecular species are higher in MBV compared to those in liquid-phase EV. Content of free PUFA (FIG. 6A) and their oxygenated metabolites (FIG. 6B) in parent cell, liquid phase EV and MBV was assessed. Data are presented as mean±s.d., *p<0.05 and compared to cells or MBV, with n=3. FIG. 6C shows the contents of singly-, doubly- and triply-oxygenated phospholipid species in parent cells, liquid phase EV, and MBV. The data are presented as heat maps autoscaled to Z-scores and coded blue (low values) to red (high values). Abbreviations are: EV, exosomal vesicles; MBV, matrix-bound vesicles; PL, phospholipids; PC, phosphatidylcholine; PE, phosphatidylethanolamine, PI, phosphatidylinositol; PS, phosphatidylserine; BMP, bis-monoacylglycerophosphate; PA, phosphatidic acid; PG, phosphatidylglycerol; and CL, cardiolipin.

FIG. 7 depicts routes of administration of MBV for treatment of rheumatoid arthritis in rat animal models.

FIGS. 8A-8E show the individual arthritis scores of control and arthritis rat models treated with Pristane-only, intraperitoneal (IP) methotrexate (MTX), periarticular (PA) MBV, or intravenous (IV) MBV. FIG. 8A shows arthritis scores across treatment groups at day 7, FIG. 8B shows arthritis scores across treatment groups at day 10, FIG. 8C shows arthritis scores across treatment groups at day 13, FIG. 8D shows arthritis scores across treatment groups at day 17, and FIG. 8E shows arthritis scores across treatment groups at day 21

FIG. 9A shows photographs taken from multiple views of Sprague-Dawley rats induced to phenocopy clinical arthritis through Pristane and treated with Pristane-only, IP methotrexate, PA MBV, or IV MBV. FIG. 9B shows a closer view of disease control and periarticular MBV treated rat paws.

FIG. 10 shows the average arthritis scores over the first 21 days of treatment in the control and arthritis rat models treated with Pristane-only, IP methotrexate, PA MBV, or IV MBV.

FIG. 11A shows digital images taken of paws of control and arthritis rat models treated with IP methotrexate, PA MBV, and IV MBV. FIG. 11B shows the average arthritis scores over the first 77 days of treatment in the arthritic-rat model treated with IP methotrexate, PA MBV, and IV MBV.

FIGS. 12A-12E show that local and systemic administration of MBV significantly reduce both acute and chronic pristane-induced arthritis disease severity. A) Experimental design and treatment regimen. B) Representative images of fore- and hind-paws for each group at day 0 and day 100. Substantial edema, erythema, and distortion of the fore- and hind-paw is evident in the Pristane+PBS group with no superficial changes observed in the remaining treatment groups. C) Intra-peritoneal methotrexate significantly reduces disease scoring between days 10-21 and days 84-100 compared to Pristane+PBS (p<0.05). D) Peri-articular MBV significantly reduces disease scoring between days 10-21 and days 70-100 compared to Pristane+PBS (p<0.05). E) Intravenous MBV significantly reduces disease scoring between days 10-21 and days 70-100 compared to Pristane+PBS (p<0.05). All values represented in panels C-E are Mean±SEM and n=8 for days 7-28 and n=4 days 28-100.

FIGS. 13A-13E provide evidence that matrix-bound nanovesicles reduce synovial inflammation, cartilage degradation, and proteoglycan loss in chronic pristane-induced arthritis. A) Representative 10× H&E and 10× Toluidine blue images of the tibiotalar (tibia=Ti, talus=Ta) joint and 40× H&E images of the adjacent synovium (syn). B) On examination of 40× H&E images of the synovium, i.p. MTX, p.a. MBV, and i.v. MBV all significantly reduced synovial inflammation and relative proportion of infiltrating immune cells (p<0.05). C) On examination of 10× Toluidine blue images, i.p. MTX, p.a. MBV, and i.v. MBV reduced the severity of cartilage damage compared to Pristane+PBS. D) On examination of 10× H&E images, i.p. MTX, p.a. MBV, and i.v. MBV reduced the severity of proteoglycan loss compared to Pristane+PBS. E) Pristane+I.p. MTX, +p.a. MBV, and +i.v. MBV all significantly reduced the cumulative histopathologic score compared to Pristane+PBS (p<0.05). All values in panels 13B-13E represent mean±SEM (n=3).

FIGS. 14A-14C document that matrix-bound nanovesicles modulate joint macrophage phenotype from an M1-predominant phenotype seen in pristane induced arthritis towards an M2-predominant phenotype. A) Representative 20× IHC images of synovial tissue adjacent to the tibiotalar joint. Composite images of the four channels are pictured as well as individual color channels for each target (nucleus, CD68, TNFalpha, and CD206). B) Compared to Control+PBS, Pristane+PBS significantly increases the ratio of M1 macrophages (TNFalpha+/CD68+) compared to M2 macrophages (CD206+/CD68+) (p<0.05). All three treatment groups (i.p. MTX, p.a. MBV, and i.v. MBV) significantly reduce the ratio of M1:M2 macrophages in the synovial tissue (p<0.05). C) There is an increase in the ratio of M2:M1 macrophages in all three treatment groups compared to Control+PBS and Pristane+PBS (p>0.05). All values in panels B and C represent mean±SEM (n=3).

FIGS. 15A-15B are digital images showing that systemic and local administration of matrix-bound nanovesicles prevent adverse bone remodeling in chronic pristane-induced arthritis by microCT imaging. A) Representative microCT images of forepaws at day 100. Substantial bone remodeling and damage is seen in the Pristane+PBS group throughout the proximal bones of the forepaw while all three treatment groups show minimal to absent changes in bone morphology. B) Representative microCT images of hindpaws at day 100. Substantial bone remodeling and damage is seen in the Pristane+PBS group localized to the tibiotalar joint while all three treatment groups show minimal to absent changes in bone morphology, especially in the region of the tibiotalar joint.

FIG. 16 are digital images showing treatment of Imiquimod-induced murine psoriasis with matrix-bound nanovesicles. Images are of dorsal skin that is shaved, depilated, and treated with either IMQ (control 1) or petroleum jelly (control 2). Systemic administration of MBV reduces erythema and scaling in imiquimod-induced psoriasis. Additionally, systemic administration of MBV reduces skin thickness in imiquimod-induced psoriasis.

FIG. 17 is a line graph of cumulative psoriasis scoring. Cummulative psoriasis scoring, as derived from the Psoriasis Areas and Severity index (PASI) was used to score disease severity. Systemic administration of MBV (by intraperitoneal administration) substantially reduced cumulative psoriasis scoring in the IMQ-induced psoriasis model of cutaneous inflammation. Cumulative scoring was based on the modified clinical Psoriasis Area and Severity Index (PASI) (Maximum score=12). Erythema and scaling was scored independently on a scale from 0-4: 0, none; 1, slight; 2, moderate; 3, marked; and 4, very marked. The thickness of both ears was measured using a caliper, and the percentage change from baseline was calculated as an indicator of skin thickness. Systemic administration of MBV reduces overall disease score compared to imiquimod-induced psoriasis.

FIG. 18 is a bar graph of the results from a Keyhole Limpet Hemocyanin IgM Assay after MBV. Systemic administration of MBV before immunization with KLH does not affect the ability of a host animal to mount a normal IgM antibody response to the KLH antigen. Cyclophosphamide, a known immunosuppressant significantly reduces anti-KLH IgM levels at days 7, 14, and 21 compared to the Vehicle+KLH control. There is no significant difference between the Vehicle+KLH control and the MBV treated animals in the level of IgM produced. These results demonstrate that systemic administration of MBV does not suppress a physiologic antibody immune response.

FIG. 19 is a bar graph of FIG. 8 the results from a Keyhole Limpet Hemocyanin IgG Assay after MBV. Systemic administration of MBV before immunization with KLH does not affect the ability of a host animal to mount a normal IgG antibody response to the KLH antigen. Cyclophosphamide, a known immunosuppressant significantly reduces anti-KLH IgG levels at days 7, 14, and 21 compared to the Vehicle+KLH control. There is no significant difference between the Vehicle+KLH control and the MBV treated animals in the level of IgG produced. These results demonstrate that systemic administration of MBV does not suppress a physiologic antibody immune response.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Oct. 22, 2020, 1.097 bytes], which is incorporated by reference herein.

### DETAILED DESCRIPTION

Biologic scaffolds composed of extracellular matrix (ECM) have been developed as surgical mesh materials and are used in clinical applications including ventral hernia repair (Alicuban et al., Hernia. 2014; 18(5):705-712), musculoskeletal reconstruction (Mase et al., Orthopedics. 2010; 33(7):511), esophageal reconstruction (Badylak et al., Tissue Eng Part A. 2011; 17(11-12):1643-50), dura mater replacement (Bejjani et al., J Neurosurg. 2007; 106(6):1028-1033), tendon repair (Longo et al., Stem Cells Int. 2012; 2012:517165), breast reconstruction (Salzber, Ann Plast Surg. 2006; 57(1):1-5), amongst others (Badylak et al., Acta Biomater. 2009; 5(1):1-13).

Matrix bound nanovesicles (MBV) are embedded within the fibrillar network of the ECM. These nanoparticles shield their cargo from degradation and denaturation during the ECM-scaffold manufacturing process.

Exosomes are vesicles that previously have been identified almost exclusively in body fluids and cell culture supernatant. It has been demonstrated that MBV and exosomes are distinct. MBV differ from other vesicles, for example, as they are resistant to detergent and/or enzymatic digestion, have a unique lipid profile, contain a cluster of different microRNAs. MBV do not have the same characteristic surface proteins found in other vesicles, such as exosomes.

As disclosed herein, MBV modulate a systemic healing response (such as through systemic administration), for example, to preserve or to restore biological function. For example, administration of MBV may be to preserve or to restore immune response, such as to treat an autoimmune disorder (such as rheumatoid arthritis, scleroderma, ulcerative colitis, pemphigus, pemphigoid, Crohn's disease, psoriasis, psoriatic arthritis, sclerosis, or systemic lupus erythematosus).

### Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural subjects and is considered equivalent to the phrase “comprising at least one subject.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GENBANK® Accession Nos. referred to herein are the sequences available at least as early as Sep. 16, 2015. All references, patent applications and publications, and GENBANK® Accession numbers cited herein are incorporated by reference. Unless otherwise indicated, “about” indicates within five percent. In order to facilitate review of the various embodiments of the disclosure, the following explanations of specific terms are provided:

Administration: The introduction of a composition (such as MBV or a pharmaceutical preparation that includes MBV) into a subject by a chosen route. The route can be local or systemic. For example, if the chosen route is intravenous, the composition is administered by introducing the composition into a vein of the subject. If the chosen route is local, the composition can be administered by introducing the composition directly into a tissue of the subject.

Animal: Living multi-cellular vertebrate organisms, a category that includes, for example, mammals and birds. The term “mammal” includes both human and non-human mammals. Similarly, the term “subject” includes both human and veterinary subjects.

Arthritis: Arthritis is a disease that affects the synovial membranes of one or more joints in the body. It is the most common type of joint disease and is characterized by inflammation of the joint. The disease is usually oligoarticular (affects few joints), but may be generalized. The joints commonly involved include the hips, knees, lower lumbar and cervical vertebrae, proximal and distal interphalangeal joints of the fingers, first carpometacarpal joints, and first tarsometatarsal joints of the feet. Symptoms include joint pain and stiffness, redness, warmth, swelling, and decreased range of motion of the affected joints. In some embodiments, the compositions and methods disclosed herein can be used to treat arthritis.

One type of arthritis is rheumatoid arthritis. Rheumatoid arthritis is a chronic, systemic, autoimmune disease that affects the synovial membranes of multiple joints in the body. Because the disease is systemic, there are many extra-articular features of the disease. For example, neuropathy, scleritis, lymphadenopathy, pericarditis, splenomegaly, arteritis, and rheumatoid nodules are frequent components of the disease. In most cases of rheumatoid arthritis, the subject has remissions and exacerbations (also referred to as “flares or flare-ups”) of the symptoms. Rheumatoid arthritis is considered an autoimmune disease that is acquired and in which genetic factors appear to play a role. In some embodiments, the compositions and methods disclosed herein can be used to treat rheumatoid arthritis.

Another type of arthritis is psoriatic arthritis, a seronegative spondyloarthropathy, which is a long-term form of autoimmune arthritis that occurs in people affected by psoriasis. Psoriatic arthritis presents as swelling of entire fingers and toes with a sausage-like appearance, which can be associated with changes to the nails (such as small depressions in the nail, thickening of the nails, and detachment of the nail from the nailbed), skin changes consistent with psoriasis (such as red, scaly, and itchy plaques, which frequently occur before onset of psoriatic arthritis). Psoriatic arthritis affects up to 30% of people with psoriasis and occurs in both children and adults. Various types of psoriatic arthritis are included, such as oligoarticular, polyarticular, arthritis mutilans, arthritis mutilans, spondyloarthritis, and distal interphalangeal predominant. Treatment can include NSAIDs (such as ibuprofen, naproxen, diclofenac, indomethacin, and etodolac), disease-modifying antirheumatic drugs (DMARDs, such as methotrexate, leflunomide, cyclosporin, azathioprine, and sulfasalazine), biological response modifiers (such as TNF-α inhibitors, including infliximab, etanercept, golimumab, certolizumab pegol, and adalimumab; the IL-12/IL-23 inhibitor ustekinumab; and the Jak inhibitor tocifitinib, or XELJANZ®), phosphodiesterase-4 inhibitors (such as apremilast), low level laser therapy, retinoid etretinate, photochemotherapy with methoxy psoralen and long-wave ultraviolet light (PUVA), joint injections with corticosteroids, and orthopedic surgery (such as joint replacement). In some embodiments, the compositions and methods disclosed herein can be used to treat psoriatic arthritis.

Autoimmune disorder: Autoimmune disorders include a broad range of related diseases in which a person's immune system produces an inappropriate response (e.g., a B cell or a T cell response) against an endogenous antigen, with consequent injury to its own cells, tissues, and/or organs, resulting in inflammation and damage. There are over 80 different autoimmune diseases. The injury may be localized to certain organs or tissue, such as Sjogren's disease, or may be systemic, such as psoriasis. Although symptoms can vary with type of autoimmune disease, common symptoms include fatigue, achy muscles, swelling and redness, low-grade fever, trouble concentrating, numbness and tingling in the hands and feet, hair loss, and skin rashes. Tests for autoimmune disorders similarly vary with types, but typical tests include an antinuclear antibody test (ANA) and tests for specific autoantibodies produced in certain disorder types as well as an examination for inflammation in the body.

In some examples, autoimmune diseases include Addison's disease, alopecia areata, ankylosing spondylitis, anti-phospholipid antibody syndrome, autoimmune hepatitis, autoimmune encephalitis, Celiac disease, Crohn's disease, Goodpasture's Syndrome, Grave's disease, Guillain-Barre syndrome, Hashimoto's thyroiditis, immune thrombocytopenia, IgA Nephropathy, inflammatory bowel disease (IBD), multiple sclerosis, myasthenia gravis, pemphigoid, pemphigus, polyglandular autoimmune syndrome type 2, psoriasis, psoriatic arthritis, rheumatoid arthritis, scleroderma, Sjogren's syndrome, systemic lupus erythematosus, Takayasu's arteriosis, type 1 diabetes, ulcerative colitis, or undifferentiated connective tissue disease (UCTD).

A variety of treatments for autoimmune diseases can be administered. In some examples, anti-inflammatories and/or immunosuppressing drugs can be administered.

Biocompatible: Any material, that, when implanted in a mammalian subject, does not provoke an adverse response in the subject. A biocompatible material, when introduced into an individual, is able to perform its' intended function, and is not toxic or injurious to that individual, nor does it induce immunological rejection of the material in the subject.

Autoimmune Encephalitis: Inflammation of the brain with varying severity, due to autoimmune disease. Symptoms can include headache, fever, confusion, a stiff neck, and vomiting, and complications can include seizures, hallucinations, trouble speaking, memory problems, and problems with hearing. Various types of autoimmune encephalitis are included, such as antibody-mediated anti-N-methyl-D-aspartate-receptor encephalitis (anti-NMDA receptor encephalitis, which can be accompanied by ovarian teratoma and mostly affects women 18-45 years of age) and Rasmussen encephalitis. Other autoimmune diseases can cause autoimmune encephalitis, such as systemic lupus erythematosus, Hashimoto's encephalopathy, autoimmune limbic encephalitis, and Sydenham's chorea. In some embodiments, the compositions and methods disclosed herein can be used to treat autoimmune encephalitis.

Enriched: A process whereby a component of interest, such as a nanovesicle, that is in a mixture has an increased ratio of the amount of that component to the amount of other undesired components in that mixture after the enriching process as compared to before the enriching process.

Extracellular matrix (ECM): A complex mixture of structural and functional biomolecules and/or biomacromolecules including, but not limited to, structural proteins, specialized proteins, proteoglycans, glycosaminoglycans, and growth factors that surround and support cells within tissues and, unless otherwise indicated, is acellular. ECM preparations can be considered to be “decellularized” or “acellular”, meaning the cells have been removed from the source tissue through processes described herein and known in the art. By “ECM-derived material,” such as an “ECM-derived nanovesicle,” “Matrix bound nanovesicle,” “MBV” or “nanovesicle derived from an ECM” it is a nanovesicle that is prepared from a natural ECM or from an in vitro source wherein the ECM is produced by cultured cells. ECM-derived nanovesicles are defined below.

Flare-up or flare: An exacerbation of a disease or disorder, such as an autoimmune disorder. A flare-up occurs when symptoms of a disease or disorder that have been present for a time suddenly worsen. For example, in a flare-up, severity of a disease or disease symptom(s) transiently worsens but eventually subside(s) or lessen(s). For example, in an autoimmune disorder, inflammation or other signs or symptoms of the autoimmune disorder can worsen during a flare-up.

Inflammatory bowel diseases (IBD): An autoimmune disease characterized by chronic, relapsing intestinal inflammation of obscure origin. In patients with IBD, ulcers and inflammation of the inner lining of the intestines lead to symptoms of abdominal pain, diarrhea, and rectal bleeding. There are two primary types of IBD, Crohn's disease (CD) and ulcerative colitis (UC); both of these diseases appear to result from the unrestrained activation of an inflammatory response in the intestine mediated by autoantibodies against intestinal epithelial cells.

The main difference between CD and UC is the location and nature of the inflammatory changes. CD can affect any part of the gastrointestinal tract, from mouth to anus (skip lesions), although a majority of the cases start in the terminal ileum. Ulcerative colitis, in contrast, is restricted to the colon and the rectum. Symptoms of IBD most commonly include fever, vomiting, diarrhea, bloody stool (hematochezia), abdominal pain, and weight loss but also may include a host of other problems. The severity of symptoms may impair the quality of life of patients that suffer from IBD. For most patients, IBD is a chronic condition with symptoms lasting for months to years. It is most common in young adults, but can occur at any age. IBD especially common in people of Jewish descent and has racial differences in incidence as well.

Diagnosis of IBD can be based on the clinical symptoms or the use of a barium enema, but direct visualization (sigmoidoscopy or colonoscopy) is the most accurate test. Protracted IBD is a risk factor for colon cancer, and treatment of IBD can involve medications and surgery.

Some patients with UC only have disease in the rectum (proctitis). Others with UC have disease limited to the rectum and the adjacent left colon (proctosigmoiditis). Yet others have UC of the entire colon (universal IBD). Symptoms of UC are generally more severe with more extensive disease (larger portion of the colon involved with disease). The prognosis for patients with disease limited to the rectum (proctitis) or UC limited to the end of the left colon (proctosigmoiditis) is better than that of full colon UC. In patients with more extensive disease, blood loss from the inflamed intestines can lead to anemia, and may require treatment with iron supplements or even blood transfusions.

Rarely, the colon can acutely dilate to a large size when the inflammation becomes very severe. This condition is called toxic megacolon. Patients with toxic megacolon are extremely ill with fever, abdominal pain and distention, dehydration, and malnutrition. Unless the patient improves rapidly with medication, surgery is usually necessary to prevent colon rupture.

CD can occur in all regions of the gastrointestinal tract. With this disease intestinal obstruction due to inflammation and fibrosis occurs in a large number of patients. Granulomas and fistula formation are frequent complications of CD. Disease progression consequences include intravenous feeding, surgery, and colostomy.

Remission of IBD can be measured in many ways. Clinical remission refers to a lack of symptoms of the disease. Histological and endoscopic remission refers to an absence of inflammation from biopsied bowel tissue removed during an endoscopic procedure.

Isolated: An “isolated” biological component (such as a nucleic acid, protein cell, or nanovesicle) has been substantially separated or purified away from other biological components in the cell of the organism or the ECM, in which the component naturally occurs. Nucleic acids and proteins that have been “isolated” include nucleic acids and proteins purified by standard purification methods. MBV that have been isolated are removed from the fibrous materials of the ECM. The term also embraces nucleic acids and proteins prepared by recombinant expression in a host cell as well as chemically synthesized nucleic acids.

Lysyl oxidase (Lox): A copper-dependent enzyme that catalyzes formation of aldehydes from lysine residues in collagen and elastin precursors. These aldehydes are highly reactive, and undergo spontaneous chemical reactions with other lysyl oxidase-derived aldehyde residues, or with unmodified lysine residues. In vivo, this results in cross-linking of collagen and elastin, which plays a role in stabilization of collagen fibrils and for the integrity and elasticity of mature elastin. Complex cross-links are formed in collagen (pyridinolines derived from three lysine residues) and in elastin (desmosines derived from four lysine residues) that differ in structure. The genes encoding Lox enzymes have been cloned from a variety of organisms (Hamalainen et al., Genomics 11:508, 1991; Trackman et al., Biochemistry 29:4863, 1990; incorporated herein by reference). Residues 153-417 and residues 201-417 of the sequence of human lysyl oxidase have been shown to be important for catalytic function. There are four Lox-like isoforms, called LoxL1, LoxL2, LoxL3 and LoxL4.

Macrophage: A type of white blood cell that phagocytoses and degrades cellular debris, foreign substances, microbes, and cancer cells. In addition to their role in phagocytosis, these cells play an important role in development, tissue maintenance and repair, and in both innate and adaptive immunity in that they recruit and influence other cells including immune cells such as lymphocytes. Macrophages can exist in many phenotypes, including phenotypes that have been referred to as M1 and M2. Macrophages that perform primarily pro-inflammatory functions are called M1 macrophages (CD86+/CD68+), whereas macrophages that decrease inflammation and encourage and regulate tissue repair are called M2 macrophages (CD206+/CD68+). The markers that identify the various phenotypes of macrophages vary among species. It should be noted that macrophage phenotype is represented by a spectrum that ranges between the extremes of M1 and M2. F4/80 (encoded by the adhesion G protein coupled receptor E1 (ADGRE1) gene) is a macrophage marker, see GENBANK® Accession No. NP_001243181.1, Apr. 6, 2018 and NP_001965, Mar. 5, 2018, both incorporated herein by reference. It is believed that MBV have the ability to modulate the phenotype of macrophages, leading to an increase in M2-like, regulatory, or pro-remodeling macrophages. Thus MBV can be used to induce an M2 phenotype in macrophages and inhibit M1 macrophages in a subject.

MicroRNA: A small non-coding RNA that is about 17 to about 25 nucleotide bases in length, that post-transcriptionally regulates gene expression by typically repressing target mRNA translation. A miRNA can function as negative regulators, such that greater amounts of a specific miRNA will correlates with lower levels of target gene expression. There are three forms of miRNAs, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nucleotide overhang at the 3′ end. The cleavage product, the premature miRNA (pre-miRNA) is about 60 to about 110 nucleotides long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nucleotides in length.

Multiple sclerosis (MS): An autoimmune disease classically described as a central nervous system white matter disorder disseminated in time and space that presents as relapsing-remitting illness in 80-85% of patients. It is a chronic, typically progressive disease involving damage to sheaths of nerve cells in the brain and spinal cord. Diagnosis can be made by brain and spinal cord magnetic resonance imaging (MRI), analysis of somatosensory evoked potentials, and analysis of cerebrospinal fluid to detect increased amounts of immunoglobulin or oligoclonal bands. MRI is a particularly sensitive diagnostic tool. MRI abnormalities indicating the presence or progression of MS include hyperintense white matter signals on T2-weighted and fluid attenuated inversion recovery images, gadolinium enhancement of active lesions, hypointensive “black holes” (representing gliosis and axonal pathology), and brain atrophy on T1-weighted studies. Serial MRI studies can be used to indicate disease progression. The status of MS patients can be evaluated by longitudinal, monthly follow-up of magnetic resonance (MRI) activity in the brain of MS patients. MRI offers a unique set of outcome measures for phase I/II clinical trials in small cohorts of patients, and is thus well suited to establish data for proof of principle for novel therapeutic strategies (e.g., see Harris et al., Ann. Neurol. 29:548-555, 1991; MacFarland et al., Ann. Neurol. 32:758-766, 1992; Stone et al., Ann. Neurol. 37:611-619, 1995).

Relapsing-remitting multiple sclerosis is a clinical course of MS that is characterized by clearly defined, acute attacks with full or partial recovery and no disease progression between attacks. During remissions, all symptoms may disappear, or some symptoms may continue and become permanent. However, there is no apparent progression of the disease during the periods of remission.

Secondary-progressive multiple sclerosis is a clinical course of MS that is initially relapsing-remitting and then becomes progressive at a variable rate, possibly with an occasional relapse and minor remission. Primary progressive multiple sclerosis presents initially in the progressive form.

Symptoms of MS include numbness, impairment of speech and muscular coordination, blurred vision, and severe fatigue.

Treatment for MS include interferon beta-la, interferon beta-Ib, glatiramer acetate, mitoxantrone, natalizumab, fingolimod, teriflunomide, dimethyl fumarate, alemtuzumab, ocrelizumab, siponimod, cladribine, ocrelizumab, rituximab, and alternative/complementary medicine. In some embodiments, the compositions and methods disclosed herein can be used to treat MS.

Nanovesicle: An extracellular vesicle that is a nanoparticle of about 10 to about 1,000 nm in diameter. Nanovesicles are lipid membrane bound particles that carry biologically active signaling molecules (e.g. microRNAs, proteins) among other molecules. Generally, the nanovesicle is limited by a lipid bilayer, and the biological molecules are enclosed and/or can be embedded in the bilayer. Thus, a nanovesicle includes a lumen surrounded by plasma membrane. The different types of vesicles can be distinguished based on diameter, subcellular origin, density, shape, sedimentation rate, lipid composition, protein markers, nucleic acid content and origin, such as from the extracellular matrix or secreted. A nanovesicle can be identified by its origin, such as a matrix bound nanovesicle from an ECM (see above), protein content and/or the miR content.

An “exosome” or “liquid phase extracellular vesicle (EV)” is a membranous vesicle which is secreted by a cell, and ranges in diameter from 10 to 150 nm. Generally, late endosomes or multivesicular bodies contain intralumenal vesicles which are formed by the inward budding and scission of vesicles from the limited endosomal membrane into these enclosed vesicles. These intralumenal vesicles are then released from the multivesicular body lumen into the extracellular environment, typically into a body fluid such as blood, cerebrospinal fluid or saliva, during exocytosis upon fusion with the plasma membrane. An exosome is created intracellularly when a segment of membrane invaginates and is endocytosed. The internalized segments which are broken into smaller vesicles and ultimately expelled from the cell contain proteins and RNA molecules such as mRNA and miRNA. Plasma-derived exosomes largely lack ribosomal RNA. Extra-cellular matrix derived exosomes include specific miRNA and protein components, and have been shown to be present in virtually every body fluid such as blood, urine, saliva, semen, and cerebrospinal fluid. Exosomes can express CD11c, CD63, CD81, and/or CD9, and thus can be CD11c and/or CD63+ and/or C81+ and/or CD9+. Exosomes do not have high levels of lysyl oxidase on their surface.

A “nanovesicle derived from an ECM” “matrix bound nanovesicle,” “MBV” or an “ECM-derived nanovesicle” all refer to the same membrane bound particles, ranging in size from 10 nm-1000 nm, present in the extracellular matrix, which contain biologically active signaling molecules such as protein, lipids, nucleic acid, growth factors and cytokines that influence cell behavior. The terms are interchangeable, and refer to the same vesicles. These nanovesicles are embedded within, and bound to, the ECM and are not simply attached to the surface or circulating freely in body fluids. These nanovesicles are resistant to harsh isolation conditions, such as freeze-thawing and digestion with proteases such as pepsin, elastase, hyaluronidase, proteinase K, and collagenase, and digestion with detergents. MBV are distinct from other extracellular vesicles including exosomes and have a phospholipid composition distinct from exosomes. In certain circumstances, MBV can also be distinguished from exosomes based on the absence of certain markers commonly attributed to exosomes. In some embodiments, MBV are characterized by one or more of the following features of protein expression or lipid content:
(i) MBV may not express one or more of CD63 and/or CD81 and/or CD9 or have low or barely detectable levels of CD63 and/or CD81 and/or CD9 (CD63^{lo} and/or CD81^{lo} and/or CD9^{lo})(see, e.g., Example 1) compared with other vesicles, such as exosomes. A variety of methods can be used to distinguish low, barely detectable, or absent expression of CD63 and/or CD81 and/or CD9 in MBV, for example, antibody-based methods, such as western blotting or flow cytometry (see, e.g., Bashashati and Brinkman, Adv Bioinformatics, 2009: 584603). In some embodiments, MBV expression of CD63 and/or CD81 and/or CD9 is considered low or barely detectable compared with other vesicles where the expression of CD63 and/or CD81 and/or CD9 in MBV is at least one standard deviation or at least two standard deviations below the mean expression of other vesicles, such as exosomes;
(ii) MBV have a phospholipid content wherein at least 55% of total phospholipids comprise phosphatidylcholine (PC) and phosphatidyl inositol (PI) in combination;
(iii) MBV have a phospholipid content wherein 10% or less of total phospholipids comprise sphingomyelin (SM);
(iv) MBV have a phospholipid content wherein 20% or less of total phospholipids comprise phosphatidylethanolamine (PE);
(v) MBV have a phospholipid content wherein 15% or greater of the total phospholipid content comprises phosphatidylinositol (PI) with the percent representing the percent of lipid concentration.

In some embodiments, MBV are characterized by all of the following features:
(i) do not express one or more of CD63 and/or CD81 and/or CD9 or have low or barely detectable levels of CD63 and/or CD81 and/or CD9 (CD63^{lo} and/or CD81^{lo} and/or CD9^{lo}) (as further described above);
(ii) a phospholipid content wherein at least 55% of total phospholipids comprise phosphatidylcholine (PC) and phosphatidyl inositol (PI) in combination;
(iii) a phospholipid content wherein 10% or less of total phospholipids comprise sphingomyelin (SM);
(iv) a phospholipid content wherein 20% or less of total phospholipids comprise phosphatidylethanolamine (PE); and
(v) a phospholipid content wherein 15% or greater of the total phospholipid content is phosphatidylinositol (PI).

In some embodiments, MBV are characterized by all of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

In some embodiments, MBV are characterized by one or more of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

The ECM from which MBV are isolated can be an ECM from a tissue, can be produced from cells in culture, or can be purchased from a commercial source.

Pemphigus: An autoimmune disease that affects the skin and mucous membranes. Autoantibodies form against desmoglein, which forms the attachments between adjacent epidermal cells through desmosomes. The autoantibodies attack desmogleins, which separates cells and the epidermis (“acantholysis”), forming blisters that slough off and turn into sores; the blisters can cover a significant area of the skin. A variety of pemphigus types are included, such as pemphigus vulgaris, pemphigus foliaceus, intraepidermal neutrophilic IgA dermatosis, paraneoplastic pemphigus, and Endemic pemphigus foliaceus. Treatment for pemphigus includes topical steroids (such as clobetasol), intralesional injection of steroids (such as dexamethasone), immunosuppressant drugs (such as CELLCEPT® or mycophenolic acid), serum or plasma pooled products (such as intravenous gamma globulin (IVIG), especially for severe pemphigus, for example, paraneoplastic pemphigus), and biologics (such as Rituxan®, or rituximab, especially for severe cases of recalcitrant pemphigus vulgaris). In some embodiments, the compositions and methods disclosed herein can be used to treat pemphigus.

Pemphigoid: A rare autoimmune disease that presents as blistering skin. Pemphigoid appears similar pemphigus but does not include acantholysis. Pemphigoid is more common in women and in people over 60 years old. Various types pf pemphigoid are included, such as IgG-mediated pemphigoid, for example, gestational, bullous, and cicatricial pemphigoid, as well as IgA-mediated pemphigoid, for example, IgA-mediated immunobullous diseases. Treatment can include Rituxan® or rituximab, corticosteroids (such as topical corticosteroids and systemic corticosteroids), glucocorticoid-sparing drugs, immunosuppressive drugs, anti-inflammatory drugs, biologic therapy, and intravenous immunoglobulin. In some embodiments, the compositions and methods disclosed herein can be used to treat pemphigoid.

Pharmaceutically acceptable carriers: The pharmaceutically acceptable carriers useful in the claimed pharmaceutical preparations are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical preparations to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition capable of inducing a desired therapeutic or prophylactic effect when properly administered to a subject or a cell.

Phospholipid: A class of lipids having a structure consisting of two hydrophobic fatty acid tails and a hydrophilic head consisting of a phosphate group. Major classes of phospholipids include phosphatidylcholine (PC), phosphatidylethanolamine (PE), phosphatidylinositol (PI), phosphatidylserine (PS), phosphatidylglycerol (PG), sphingomyelin (SM), cardiolipin (CL), phosphatidic acid (PA), and bis-monoacylglycerophosphate (BMP). Phospholipids can be measured in a variety of ways. For example, LC-MS based global lipidomics and redox lipidomics can be used. In some embodiments, specific phospholipid content is indicated as the percent concentration of the total phospholipids (such as total phospholipids in MBV), where the percent concentration is weight/weight.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Psoriasis: An autoimmune disease characterized by patches of abnormal skin, such as red or purple, dry, itchy, and scaly patches, and abnormally excessive and rapid growth of the epidermal layer of the skin. Psoriasis symptoms can range from small, localized patches to full body coverage. Various types of psoriasis are included, such as plaque, guttate, inverse, pustular, and erythrodermic psoriasis. The underlying mechanism involves reactions of the immune system to skin cells, and treatment can include steroid creams, vitamin D3 cream, ultraviolet light, and immune system-suppressing medications, such as methotrexate. Psoriasis is associated with an increased risk of psoriatic arthritis, lymphomas, cardiovascular disease, Crohn's disease, and depression. In some embodiments, the compositions and methods disclosed herein can be used to treat psoriasis. Psoriasis flare-ups can be triggered by dry or cold weather, stress, or trauma to the skin and result in formation of the dry, itchy patches characteristic of the disorder.

Purified: The term “purified” does not require absolute purity; rather, it is intended as a relative term. Thus, for example, a purified nucleic acid molecule preparation is one in which the nucleic acid referred to is more pure than the nucleic in its natural environment within a cell. For example, a preparation of a nucleic acid is purified such that the nucleic acid represents at least 50% of the total protein content of the preparation. Similarly, a purified MBV preparation is one in which the exosome is more pure than in an environment including cells, wherein there are microvesicles and exosomes. A purified population of nucleic acids or MBV is greater than about 90%, 91%, 92%, 93%, 94%, 95%, 96%, 97%, 98%, 99% or 100% pure, or free other nucleic acids or cellular components, respectively.

Preventing or treating a disease: “Preventing” a disease refers to inhibiting the development of a disease, for example in a person who is known to have a predisposition to a disease. An example of a person with a known predisposition is someone with a history of a disease in the family, or who has been exposed to factors that predispose the subject to a condition. “Treatment” refers to a therapeutic intervention that ameliorates a sign or symptom of a disease or pathological condition after it has begun to develop.

Remission: A disease state characterized by absence of clinically detectable symptoms or signs of the disease and/or by a lack of disease progression. What constitutes remission may vary depending on the autoimmune disorder in question.

Relapse: A return of disease signs or symptoms or disease progression after a period of remission or an exacerbation of disease signs or symptoms after a period of symptom improvement.

Relapsing-remitting disease: Autoimmune disorders characterized by periods of symptom improvement and lack of disease progression (remitting phase) followed by worsening symptoms of the disorder and signs of disease progression, including increased inflammation in affected tissues. Multiple sclerosis, systemic lupus erythematosus, inflammatory bowel disease (including Crohn's and ulcerative colitis), psoriasis, and rheumatoid arthritis are examples of relapsing-remitting autoimmune disease.

Scleroderma: An autoimmune disease that may change the skin, blood vessels, muscles, and internal organs. The disease can be localized (such as to the skin) or involve other organs. Symptoms can include areas of thickened skin, stiffness, fatigue, and poor blood flow to the fingers or toes upon cold exposure. Various types of scleroderma are included, such as localized scleroderma, for example, localized morphea, morphea-lichen sclerosus et atrophicus overlap (LSA), generalized morphea, atrophoderma of pasini and pierini, pansclerotic morphea, morphea profunda, linear scleroderma, and systemic scleroderma, for example, CREST syndrome and progressive systemic sclerosis. The underlying mechanism involves abnormal growth of connective tissue likely due to the body's immune system attacking healthy tissues. The condition most often begins in middle age, and women are more often affected than men.

Treatment can include corticosteroids, methotrexate, non-steroidal anti-inflammatory drugs (NSAIDs), vasodilators (such as calcium channel blockers, alpha blockers, serotonin receptor antagonists, angiotensin II receptor inhibitors, statins, local nitrates, or iloprost), phosphodiesterase 5 inhibitors (such as sildenafil), bosentan, tetracycline, cyclophosphamide, azathioprine, endothelin receptor antagonists, prostanoids, antacids, prokinetics, angiotensin converting enzyme inhibitors, angiotensin II receptor antagonists, immunosuppressants (such as azathioprine, methotrexate, cyclophosphamide, mycophenolate, intravenous immunoglobulin, rituximab, sirolimus, alefacept, and the tyrosine kinase inhibitors imatinib, nilotinib and dasatinib), endothelin receptor antagonists, beta-glycan peptides, halofuginone, basiliximab, alemtuzumab, abatacept, and haematopoietic stem cell transplantation. In some embodiments, the compositions and methods disclosed herein can be used to treat scleroderma.

Subject: Human and non-human animals, including all vertebrates, such as mammals and non-mammals, such as non-human primates, mice, rabbits, sheep, dogs, cats, horses, cows, chickens, amphibians, and reptiles. In many embodiments of the described methods, the subject is a human. “Subject” is used interchangeably with the term “patient.”

Systemic lupus erythematosus (SLE): Also known as lupus, SLE is an autoimmune disease in which the body's immune system (such as anti-nuclear antibodies) mistakenly attacks healthy tissue. Symptoms range from mild to severe and can vary among subjects as well as over time, including, for example, periods of illness, or flare-ups, and periods of remission with few symptoms. Common symptoms include painful and swollen joints, fever, chest pain, hair loss, mouth ulcers, swollen lymph nodes, feeling tired, and a red rash (such as on the face). Inflammation of joints, skin, kidneys, brain, heart, or lungs may also occur. It most commonly begins between the ages of 15 and 45, but a wide range of ages can be affected. Women of childbearing age and those of African, Caribbean, and Chinese descent are at higher risk.

Treatment can include tacrolimus, disease-modifying antirheumatic drugs (DMARDs, such as corticosteroids; antimalarials, for example, hydroxychloroquine and immunosuppressants, such as methotrexate and azathioprine; hydroxychloroquine; cyclophosphamide; and mycophenolic acid), immunosuppressive drugs, analgesics (such as nonsteroidal anti-inflammatory drugs, or NSAIDs, such as indomethacin and diclofenac), opioids, intravenous immunoglobulins (IVIGs), lifestyle changes (such as avoiding sunlight and activities that induce fatigue), kidney transplantation (such as to treat lupus nephritis), and anticoagulants (such as for antiphospholipid syndrome). In some embodiments, the compositions and methods disclosed herein can be used to treat SLE.

Therapeutically effective amount: A quantity of a specific substance, such as an MBV, sufficient to achieve a desired effect in a subject being treated. When administered to a subject, a dosage will generally be used that will achieve target tissue concentrations (for example, in a bone or joint) that has been shown to achieve a desired in vitro effect.

“Total phospholipids” or “total phospholipid content”: With respect to MBV, refers to the sum of all phospholipids present in a given quantity of isolated MBV, i.e., MBV isolated from the ECM. MBV can be isolated, for example, by enzymatic digestion of decellularized ECM and differential centrifugation. The total phospholipid content can be determined by methods such as LC-MS based global lipidomics and redox lipidomics. The total phospholipid content is measured by weight. A percentage of the total phospholipid content refers to a percent concentration on a weight/weight basis.

Transplanting: The placement of a biocompatible substrate, such as an MBV, into a subject in need thereof.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, or improving a subject's physical or mental well-being. The treatment may be assessed by objective or subjective parameters; including the results of a physical examination, neurological examination, or psychiatric evaluations.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### Overview

It is disclosed herein that MBV have the capacity to treat autoimmune disorders when administered to a subject suffering from such a disorder. In particular, it has been discovered that MBV delivered systemically have a therapeutic effect in treating symptoms of autoimmune disorders commensurate with the therapeutic effect achieved from local administration of MBV to an affected tissue. Accordingly, this positions MBV as a unique systemic therapy for autoimmune disorders.

It is also disclosed herein that, for some conditions, such as, but not limited to psoriasis, local administration of MBV are particularly effective. For example, in the treatment of psoriasis, topical cutaneous administration of MBV is particularly effective.

As described in Example 2 below, in a rat model of rheumatoid arthritis, arthritis scores in rats administered MBV either systemically by tail vein intravenous injection or locally by periarticular injection improved comparably to arthritis scores in rats receiving periarticular methotrexate, the gold standard of treatment for rheumatoid arthritis. Nevertheless, it was a surprising discovery that arthritis score improvements were comparable between rats whether receiving systemic injection or local injection. It was theorized that systemic injection of MBV would result in a dilution effect and therefore not result in any significant level of localized therapeutic effect, but the opposite was seen. Accordingly, systemic administration of MBV has therapeutic potential to treat many autoimmune disorders that are not localized to one part of the body or that are not amenable to local treatment. Many autoimmune disorders cause systemic inflammation or are systemic disorders affecting many tissues. Therefore, local treatment is not an effective or efficient method of treatment. In a disorder, such as pemphigus, which affects the skin, local administration, such as topical administration, may not be practical if a significant area of the skin is affected by the disorder, and in many cases, local application to the skin may not be feasible by an affected subject if they cannot reach an affected area to locally administer a treatment, and therefore systemic treatment options could be more efficient and practical. Further, in a disease like rheumatoid arthritis, injection of therapeutic agents locally into the affected joints is extremely painful and multiple joints in various parts of the body may be affected. Therefore, an option for a systemic therapy provides not only a more comfortable treatment option, but also a more efficient mechanism for treating systemic inflammation caused by autoimmune disorders.

While local administration is contemplated for treating the autoimmune disorders disclosed herein, subjects having these disorders experience unique therapeutic benefits when MBV are administered as a systemic therapy, especially when the disorder affects sensitive tissue not accessible to local administration or not amenable to treatment by local administration for other reasons, or where the disorder affects locations and tissues throughout the body (e.g., systemic disorders). In treating autoimmune disorders where symptoms are found in more than one location in the body, systemic therapy with MBV provides an efficient and effective treatment for such disorders. Intravenous systemic administration may be used to achieve the aforementioned therapeutic effects although other methods of systemic administration are contemplated.

Disclosed herein are methods of treating an autoimmune disorder (such as a chronic autoimmune disorder) in a subject in need thereof that include administering to the subject a pharmaceutical preparation that includes a therapeutically effective amount of isolated matrix bound vesicles (MBV) derived from extracellular matrix (such as MBV derived from extracellular matrix of urinary bladder, small intestine, heart, dermis, liver, kidney, uterus, brain, blood vessel, lung, bone, muscle, pancreas, stomach, spleen, colon, adipose tissue, or esophagus, for example, MBV are derived from urinary bladder matrix (UBM), small intestinal submucosa (SIS), or urinary bladder submucosa (UBS), such as from mammalian vertebrate selected from a human, monkey, pig, cow, or sheep), thereby treating the autoimmune disorder. In some embodiments, administration is systemic.

In some embodiments, the autoimmune disorder is a non-ocular autoimmune disorder. In some embodiments, the autoimmune disorder is not rheumatoid arthritis, scleroderma, or ulcerative colitis. In some embodiments, the autoimmune disorder is Addison's disease, alopecia areata, ankylosing spondylitis, anti-phospholipid antibody syndrome, autoimmune encephalitis, autoimmune hepatitis, Celiac disease, Crohn's disease, Goodpasture's Syndrome, Grave's disease, Guillain-Barre syndrome, Hashimoto's thyroiditis, immune thrombocytopenia, IgA Nephropathy, inflammatory bowel disease (IBD), multiple sclerosis, myasthenia gravis, pemphigoid, pemphigus, polyglandular autoimmune syndrome type 2, psoriasis, psoriatic arthritis, rheumatoid arthritis, scleroderma, Sjogren's syndrome, systemic lupus erythematosus, Takayasu's arteriosis, type 1 diabetes, ulcerative colitis, or undifferentiated connective tissue disease (UCTD). In other embodiments, the autoimmune disorder is Addison's disease, alopecia areata, ankylosing spondylitis, anti-phospholipid antibody syndrome, autoimmune encephalitis, autoimmune hepatitis, Celiac disease, Crohn's disease, Goodpasture's Syndrome, Grave's disease, Guillain-Barre syndrome, Hashimoto's thyroiditis, immune thrombocytopenia, IgA Nephropathy, multiple sclerosis, myasthenia gravis, pemphigoid, pemphigus, polyglandular autoimmune syndrome type 2, psoriasis, psoriatic arthritis, Sjogren's syndrome, systemic lupus erythematosus, Takayasu's arteriosis, Type 1 diabetes, or undifferentiated connective tissue disease (UCTD).

In some embodiments, the autoimmune disorder is rheumatoid arthritis. In a specific non-limiting example, the administration is systemic. In other embodiments, the autoimmune disorder is scleroderma. In more embodiments, the autoimmune disorder is ulcerative colitis. In further embodiments, the autoimmune disorder is pemphigus. In some embodiments the autoimmune disorder is pemphigoid. In other embodiments, the autoimmune disorder is Crohn's disease. In further embodiments, the autoimmune disorder is psoriasis. In more embodiments, the autoimmune disorder is psoriatic arthritis. In yet other embodiments the autoimmune disorder is multiple sclerosis. In some embodiments, the autoimmune disorder is systemic lupus erythematosus. In some embodiments, the autoimmune disorder is autoimmune encephalitis.

In any of these embodiments, the method can include selecting the subject for treatment. The administration can be systemic or local.

In the methods of treating autoimmune disorders disclosed herein, the subject can experience a therapeutic benefit for prolonged period of time after administration of the MBV. In some examples, the subject experiences a therapeutic benefit from the administration of MBV lasting a time period of at least 2 weeks, at least 3 weeks, at least 4 weeks, at least 1 month, at least 2 months, or at least 3 months. In specific non-limiting examples, the therapeutic benefit from the administration lasts at least one week. In specific examples, the therapeutic benefit from the administration last at least two weeks. In specific non-limiting examples, the therapeutic benefit from the administration lasts at least one month. In specific non-limiting examples, the therapeutic benefit from the administration lasts at least two months. In specific examples, the therapeutic benefit from the administration last at least three months or more. In some non-limiting examples, the therapeutic benefit is a reduction in a symptom of the disease or disorder present at the time of administration. In some non-limiting examples, the therapeutic benefit is a reduction in the level of inflammation caused by the autoimmune disorder in the subject over the level of inflammation caused by the autoimmune disorder prior to administration of the MBV. In some non-limiting examples, the therapeutic benefit is a remission of the disease or disorder. In some non-limiting examples, the therapeutic benefit is a reduction in flare-ups of the disease or disorder's symptoms or elimination of flare-ups of the disease or disorder's symptoms during the time period. In some embodiments, the disclosed methods reduce flare ups of the disease, such as, but not limited to, rheumatoid arthritis, multiple sclerosis or systemic lupus erythematosus.

In some embodiments, administration can be systemic. Systemic administration can be intravenous administration, oral administration, enteral administration, parenteral administration, intranasal administration, rectal administration, sublingual administration, buccal administration, sublabial administration, intraperitoneal administration, subcutaneous, or intramuscular administration. In specific non-limiting examples, the systemic administration is intravenous administration. In specific non-limiting examples, the systemic administration is oral administration. In specific non-limiting examples, the systemic administration is enteral administration. In specific non-limiting examples, the systemic administration is parenteral administration. In specific non-limiting examples, the systemic administration is intranasal administration. In specific non-limiting examples, the systemic administration is rectal administration. In specific non-limiting examples, the systemic administration is sublingual administration. In specific non-limiting examples, the systemic administration is buccal administration. In specific non-limiting examples, the systemic administration is sublabial administration. In specific non-limiting examples, the systemic administration is intraperitoneal administration. In specific non-limiting examples, the systemic administration is subcutaneous administration. In specific non-limiting examples, the systemic administration is intramuscular administration.

### Nanovesicles Derived from an Extracellular Matrix (ECM)

Nanovesicles derived from ECM (also called matrix bound nanovesicles, MBV) are generally described in PCT Publication No. WO 2017/151862 and WO2018/204848, which are incorporated herein by reference. It is disclosed that nanovesicles are embedded in the extracellular matrix. These MBV can be isolated and are biologically active. Thus, these MBV can be used for therapeutic purposes, either alone or with another ECM. An extracellular matrix is a complex mixture of structural and functional biomolecules and/or biomacromolecules including, but not limited to, structural proteins, specialized proteins, proteoglycans, glycosaminoglycans, and growth factors that surround and support cells within mammalian tissues and, unless otherwise indicated, is acellular. Generally, the disclosed MBV are embedded in any type of extracellular matrix (ECM), and can be isolated from this location. Thus, MBV are not detachably present on the surface of the ECM, and are not exosomes.

Extracellular matrices are disclosed, for example and without limitation, in U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666; each of which is incorporated by reference in its entirety). However, an ECM can be produced from any tissue, or from any in vitro source wherein the ECM is produced by cultured cells and comprises one or more polymeric components (constituents) of native ECM. ECM preparations can be considered to be “decellularized” or “acellular”, meaning the cells have been removed from the source tissue or culture.

In some embodiments, the ECM is isolated from a vertebrate animal, for example, from a mammalian vertebrate animal including, but not limited to, human, monkey, pig, cow, sheep, etc. The ECM may be derived from any organ or tissue, including without limitation, urinary bladder, intestine (such as small intestine or large intestine), heart, dermis, liver, kidney, uterus, brain, blood vessel, lung, bone, muscle, pancreas, stomach, spleen, colon, adipose tissue, or esophagus. In specific non-limiting examples, the extracellular matrix is isolated from esophageal tissue, urinary bladder (such as urinary bladder matrix or urinary bladder submucosa), small intestinal submucosa, dermis, umbilical cord, pericardium, cardiac tissue, or skeletal muscle. The ECM can comprise any portion or tissue obtained from an organ, including, for example and without limitation, submucosa, epithelial basement membrane, tunica propria, etc. In one non-limiting embodiment, the ECM is isolated from urinary bladder.

The ECM may or may not include the basement membrane. In another non-limiting embodiment, the ECM includes at least a portion of the basement membrane. The ECM material may or may not retain some of the cellular elements that comprised the original tissue such as capillary endothelial cells or fibrocytes. In some embodiments, the ECM contains both a basement membrane surface and a non-basement membrane surface.

In one non-limiting embodiment, the ECM is harvested from porcine urinary bladders (also known as urinary bladder matrix or UBM). Briefly, the ECM is prepared by removing the urinary bladder tissue from a mammal, such as a pig, and trimming residual external connective tissues, including adipose tissue. All residual urine is removed by repeated washes with tap water. The tissue is delaminated by first soaking the tissue in a de-epithelializing solution, for example and without limitation, hypertonic saline (e.g., 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. The relatively fragile epithelial basement membrane is invariably damaged and removed by any mechanical abrasion on the luminal surface. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated. After these tissues are removed, the resulting ECM consists mainly of epithelial basement membrane and subjacent tunica propria.

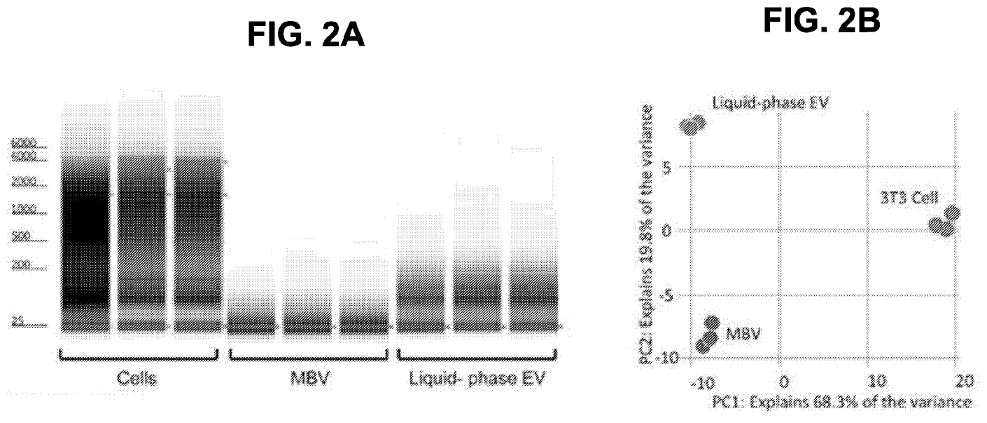

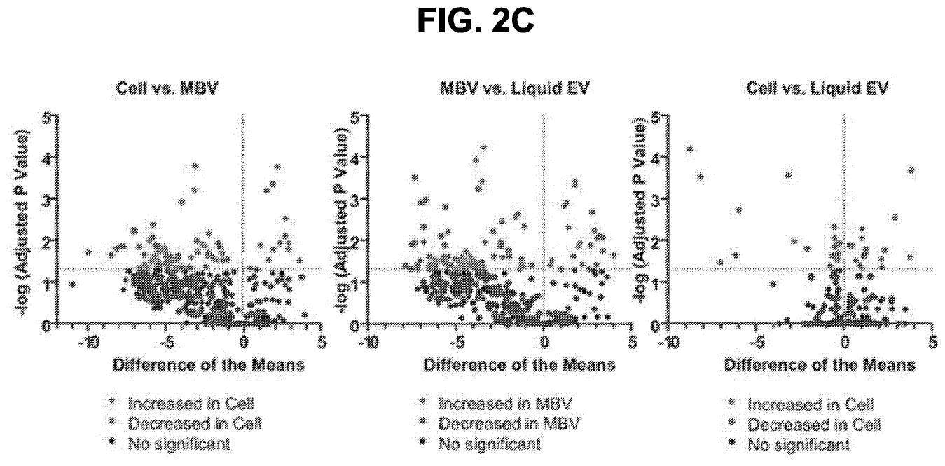

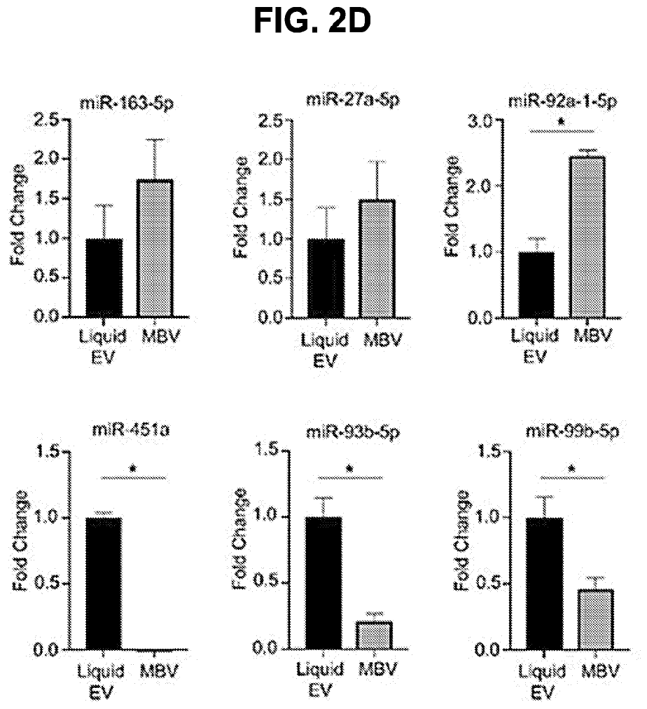

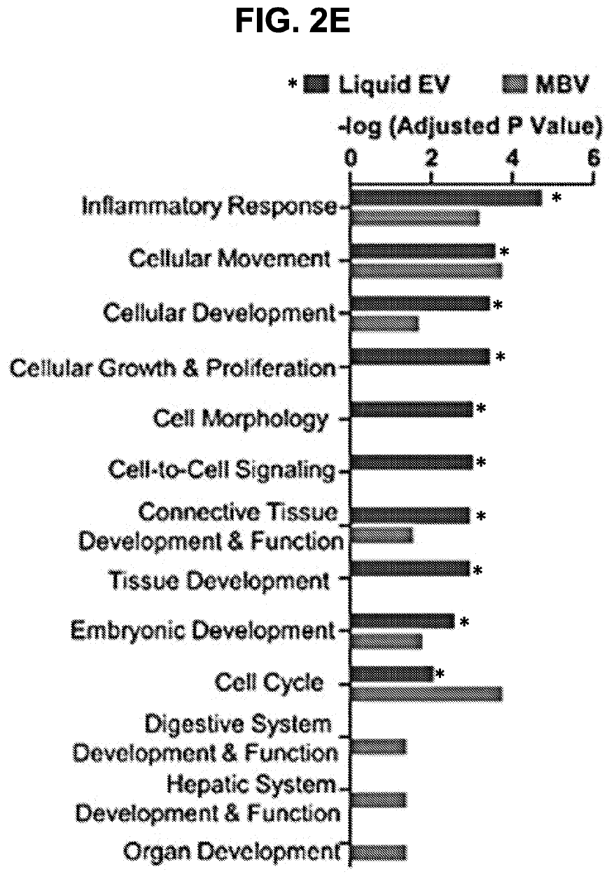

In another embodiment, the ECM is prepared by abrading porcine bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. Care is taken to prevent perforation of the submucosa. After these tissues are removed, the resulting ECM consists mainly of the tunica submucosa (see FIG. 2 of U.S. Pat. No. 9,277,999, which is incorporated herein by reference).

ECM can also be prepared as a powder. Such powder can be made according the method of Gilbert et al., Biomaterials 26 (2005) 1431-1435, herein incorporated by reference in its entirety. For example, UBM sheets can be lyophilized and then chopped into small sheets for immersion in liquid nitrogen. The snap frozen material can then be comminuted so that particles are small enough to be placed in a rotary knife mill, where the ECM is powdered. Similarly, by precipitating NaCl within the ECM tissue the material will fracture into uniformly sized particles, which can be snap frozen, lyophilized, and powdered.

In one non-limiting embodiment, the ECM is derived from small intestinal submucosa or SIS. Commercially available preparations include, but are not limited to, SURGISIS™ SURGISIS-ES™, STRATASIS™, and STRATASIS-ES™ (Cook Urological Inc.; Indianapolis, Ind.) and GRAFTPATCH™ (Organogenesis Inc.; Canton Mass.). In another non-limiting embodiment, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another embodiment, the ECM is derived from urinary bladder. Commercially available preparations include, but are not limited to UBM (ACell Corporation; Jessup, Md.).

MBV can be derived from (released from) an extracellular matrix using the methods disclosed below. Methods are disclosed for example, in Quijano et al., Tissue Engineering, part C, doi.org/10.1089/ten.TEC.2020.0243, Oct. 3, 2020, incorporated herein by reference.

In some embodiments, the ECM is digested with an enzyme, such as pepsin, collagenase, elastase, hyaluronidase, liberase or proteinase K, and the MBV are isolated. In other embodiments, the MBV are released and separated from the ECM by changing the pH with solutions such as glycine HCL, citric acid, ammonium hydroxide, use of chelating agents such as, but not limited to, EDTA, EGTA, by ionic strength and or chaotropic effects with the use of salts such as, but not limited to potassium chloride (KCl), sodium chloride, magnesium chloride, sodium iodide, sodium thiocyanate, or by exposing ECM to denaturing conditions like guanidine HCl or Urea.

In particular examples, the MBV are prepared following digestion of an ECM with an enzyme, such as pepsin, elastase, hyaluronidase, proteinase K, salt solutions, or collagenase. The ECM can be freeze-thawed, or subject to mechanical degradation.

In some embodiments, expression of CD63, CD81, and/or CD9 cannot be detected on the MBV. Thus, in some embodiments the MBV do not express CD63 and/or CD81 and/or CD9. In one specific example, CD63, CD81, and CD9 cannot be detected on the nanovesicles. In other embodiments, the MBV have barely detectable levels of CD63, CD81, and CD9, such as that detectable by Western blot. These MBV are CD63^{lo}CD81^{lo}CD9^{lo}. In other embodiments, MBV do not express detectable levels of one or more of CD63, CD81, or CD9. In other embodiments, MBV express barely detectable levels of one or more of CD63, CD81, or CD9. One of skill in the art can readily identify MBV that are CD63^{lo} and/or CD81^{lo} and/or CD9^{lo}, using, for example, antibodies that specifically bind CD63, CD81, and CD9. A low level of these markers can be established using procedures such as fluorescent activated cell sorting (FACS) and fluorescently labeled antibodies to determine a threshold for low and high amounts of CD63, CD81, and CD9. The disclosed MBV differ from nanovesicles, such as exosomes that may be transiently attached to the surface of the ECM due to their presence in biological fluids, as MBV in vivo are bound to the ECM and not found in biological fluids.

MBV have distinctive phospholipid content, for example, in comparison to exosomes. In some embodiments, the total phospholipid content of the MBV is at least 50%, 55%, 60%, 65%, 70%, 75%, 85%, or 90%, or about 50%-90%, 50%-65%, 50%-60%, 50%-70%, 60%-70%, 60%-90%, or 70%-90% of phosphatidylcholine (PC) and phosphatidyl inositol (PI) in combination. In specific embodiments, the total phospholipid content of the MBV is at least 55% of phosphatidylcholine (PC) and phosphatidyl inositol (PI) in combination. In specific embodiments, the total phospholipid content of the MBV is at least 60% of phosphatidylcholine (PC) and phosphatidyl inositol (PI) in combination. In some embodiments, the phospholipid content of the MBV comprises a phosphatidylcholine (PC) to phosphatidyl inositol (PI) ratio of less than 8:1 (for example, less than 7:1, less than 6:1, less than 5:1, less than 4:1, less than 3:1, or less than 2:1). In some embodiments, the phospholipid content of the MBV comprises a phosphatidylcholine (PC) to phosphatidyl inositol (PI) ratio in the range of 0.5-1:1, or in the range of 1:0.5-1, or in the range of 0.5-1:2, or in the range of 2:0.5-1, or in the range of 0.8-1:1, or in the range of 1:0.8-1. In one embodiment, the phospholipid content of the MBV comprises a phosphatidylcholine (PC) to phosphatidyl inositol (PI) ratio of about 1:1. In specific embodiments, the phospholipid content of the MBV comprises a phosphatidylcholine (PC) to phosphatidyl inositol (PI) ratio of about 0.9:1.

In some embodiments, the total phospholipid content of the MBV is 15%, 10%, 9%, 8%, 7%, 6%, 5%, 4% or less, or about 5%-10%, 5%-15%, 10%-15%, or 8%-12% of sphingomyelin (SM). In specific embodiments, the total phospholipid content of the MBV is 10% or less of sphingomyelin (SM). In some embodiments, the total phospholipid content of the is 15% or less of sphingomyelin (SM), 14% or less of sphingomyelin, 13% or less of sphingomyelin, 12% or less of sphingomyelin, 11% or less of sphingomyelin, 10% or less of sphingomyelin, 9% or less of sphingomyelin, 8% or less of sphingomyelin, 7% or less of sphingomyelin, 6% or less of sphingomyelin, 5% or less of sphingomyelin, or 4% or less of sphingomyelin.

In some embodiments, the total phospholipid content of the MBV 20%, 19%, 18%, 17%, 16%, 15%, 14%, 13%, 12%, 11%, or 10% or less, or about 10%-20%, 15%-20%, 14%-18%, or 12%-16% of phosphatidylethanolamine (PE). In specific embodiments, the total phospholipid content of the MBV is 20% or less of phosphatidylethanolamine (PE).

In some embodiments, the total phospholipid content of the MBV is 5%, 10%, 12%, 15%, 18%, 20%, 25%, or 30% or greater, or about 5%-30%, 10%-20%, 10-25%, 15%-25%, or 12%-18% of phosphatidylinositol (PI). In specific embodiments, MBV include a phospholipid content 15% or greater of phosphatidylinositol (PI).

In specific embodiments, the total phospholipid content of the MBV comprises 15% or more phosphatidylinositol, 20% or less phosphatidylethanolamine, and 10% or less sphingomyelin. In specific embodiments, the total phospholipid content of the MBV is 15% or more phosphatidylinositol and 20% or less phosphatidylethanolamine. In specific embodiments, the total phospholipid content of the MBV is 15% or more phosphatidylinositol and 10% or less sphingomyelin. In specific embodiments, the total phospholipid content of the MBV comprises 20% or less phosphatidylethanolamine and 10% or less sphingomyelin. In specific embodiments, the total phospholipid content of the MBV is more than 15% phosphatidylinositol, 20% or less phosphatidylethanolamine, 10% or less sphingomyelin, and at least 55% of phosphatidylinositol and phosphatidylcholine in combination. In one embodiment, the total phospholipid content of the MBV is at least 55% phosphatidylcholine (PC) and phosphatidyl inositol (PI) in combination and 10% or less sphingomyelin (SM). In specific embodiments, the total phospholipid content of the MBV is at least 55% of phosphatidylinositol and phosphatidylcholine in combination and more than 15% phosphatidylinositol. In specific embodiments, the total phospholipid content of the MBV is 55% of phosphatidylinositol and phosphatidylcholine in combination and 20% or less phosphatidylethanolamine.

The MBV may also include lysyl oxidase (Lox). Generally, nanovesicles derived from the ECM have a higher Lox content than exosomes. Lox is expressed on the surface of MBV. Nano-LC MS/MS proteomic analysis can be used to detect Lox proteins. Quantification of Lox can be performed (see, e.g., Hill R C, et al., Mol Cell Proteomics. 2015; 14(4):961-73, incorporated herein by reference in its entirety).

In certain embodiments, the MBV comprise one or more miRNA. In specific non-limiting examples, the MBV comprise one, two, or all three of miR-143, miR-145 and miR-181. MiR-143, miR-145 and miR-181 are known in the art.

The miR-145 nucleic acid sequence is provided in MiRbase Accession No. MI0000461, incorporated herein by reference. A miR-145 nucleic acid sequence is CACCUUGUCCUCACGGUCCAGUUUUCCCAGGAAUCCCUUAGAUGCUAAGAUGGGGA UUCCUGGAAAUACUGUUCUUGAGGUCAUGGUU (SEQ ID NO: 1). An miR-181 nucleic acid sequence is provided in miRbase Accession No. MI0000269, incorporated herein by reference. A miR-181 nucleic acid sequence is: AGAAGGGCUAUCAGGCCAGCCUUCAGAGGACUCCAAGGAACAUUCAACGCUGUCGG UGAGUUUGGGAUUUGAAAAAACCACUGACCGUUGACUGUACCUUGGGGUCCUUA (SEQ ID NO: 2). The miR-143 nucleic acid sequence is provided in NCBI Accession No. NR_029684.1, Mar. 30, 2018, incorporated herein by reference. A DNA encoding an miR-143 nucleic acid sequence is: GCGCAGCGCC CTGTCTCCCA GCCTGAGGTG CAGTGCTGCA TCTCTGGTCA GTTGGGAGTC TGAGATGAAG CACTGTAGCT CAGGAAGAGA GAAGTTGTTC TGCAGC (SEQ ID NO: 3).

Following administration, the MBV maintain expression of F4/80 (a macrophage marker) and CD-11b on macrophages in the subject. Nanovesicle treated macrophages are predominantly F4/80+Fizz1+indicating an M2 phenotype.

The MBV disclosed herein can be formulated into compositions for pharmaceutical delivery, and used in bioscaffolds and devices. The MBV are disclosed in PCT Publication No. WO 2017/151862, which is incorporated herein by reference.

### Isolation of MBV from the ECM

To produce MBV, ECM can be produced by any cells of interest, or can be utilized from a commercial source, see above. The MBV can be produced from the same species as, or a different species than, the subject being treated. In some embodiments, these methods include digesting the ECM with an enzyme to produce digested ECM. In specific embodiments, the ECM is digested with one or more of pepsin, elastase, hyaluronidase, collagenase a metalloproteinase, and/or proteinase K. In a specific non-limiting example, the ECM is digested with only elastase and/or a metalloproteinase. In another non-limiting example, the ECM is not digested with collagenase and/or trypsin and/or proteinase K. In other embodiments, the ECM is treated with a detergent. In further embodiments, the method does not include the use of enzymes. In specific non-limiting examples, the method utilizes chaotropic agents or ionic strength to isolate MBV such as salts, such as potassium chloride. In additional embodiments, the ECM can be manipulated to increase MBV content prior to isolation of MBV. Methods for the isolation of MBV are disclosed for example, in Quijano et al., Tissue Engineering, part C, doi.org/10.1089/ten.TEC.2020.0243, Oct. 3, 2020, incorporated herein by reference.

In some embodiments, the ECM is digested with an enzyme. The ECM can be digested with the enzyme for about 12 to about 48 hours, such as about 12 to about 36 hours. The ECM can be digested with the enzyme for about 12, about 24 about 36 or about 48 hours. In one specific non-limiting example, the ECM is digested with the enzyme at room temperature. However, the digestion can occur at about 4° C., or any temperature between about 4° C. and 25° C. Generally, the ECM is digested with the enzyme for any length of time, and at any temperature, sufficient to remove collagen fibrils. The digestion process can be varied depending on the tissue source. Optionally, the ECM is processed by freezing and thawing, either before or after digestion with the enzyme. The ECM can be treated with detergents, including ionic and/or non-ionic detergents.

The digested ECM is then processed, such as by centrifugation, to isolate a fibril-free supernatant. In some embodiments the digested ECM is centrifuged, for example, for a first step at about 300 to about 1000 g. Thus, the digested ECM can be centrifuged at about 400 g to about 750 g, such as at about 400 g, about 450 g, about 500 g or about 600 g. This centrifugation can occur for about 10 to about 15 minutes, such as for about 10 to about 12 minutes, such as for about 10, about 11, about 12, about 14, about 14, or about 15 minutes. The supernatant including the digested ECM is collected.

The MBV include Lox. In some embodiments, methods for isolating such MBV include digesting the extracellular matrix with elastase and/or metalloproteinase to produce digested extracellular matrix, centrifuging the digested extracellular matrix to remove collagen fibril remnants and thus to produce a fibril-free supernatant, centrifuging the fibril-free supernatant to isolate the solid materials, and suspending the solid materials in a carrier.

In some embodiments, digested ECM also can be centrifuged for a second step at about 2000 g to about 3000 g. Thus, the digested ECM can be centrifuged at about 2,500 g to about 3,000 g, such as at about 2,000 g, 2,500 g, 2,750 g or 3,000 g. This centrifugation can occur for about 20 to about 30 minutes, such as for about 20 to about 25 minutes, such as for about 20, about 21, about 22, about 23, about 24, about 25, about 26, about 27, about 28, about 29 or about 30 minutes. The supernatant including the digested ECM is collected.

In additional embodiments, the digested ECM can be centrifuged for a third step at about 10,000 to about 15,000 g. Thus, the digested ECM can be centrifuged at about 10,000 g to about 12,500 g, such as at about 10,000 g, 11,000 g or 12,000 g. This centrifugation can occur for about 25 to about 40 minutes, such as for about 25 to about 30 minutes, for example for about 25, about 26, about 27, about 28, about 29, about 30, about 31, about 32, about 33, about 34, about 35, about 36, about 37, about 38, about 39 or about 40 minutes. The supernatant including the digested ECM is collected.

One, two or all three of these centrifugation steps can be independently utilized. In some embodiments, all three centrifugation steps are utilized. The centrifugation steps can be repeated, such as 2, 3, 4, or 5 times. In one embodiment, all three centrifugation steps are repeated three times.

In some embodiments, the digested ECM is centrifuged at about 500 g for about 10 minutes, centrifuged at about 2,500 g for about 20 minutes, and/or centrifuged at about 10,000 g for about 30 minutes. These step(s), such as all three steps are repeated 2, 3, 4, or 5 times, such as three times. Thus, in one non-limiting example, the digested ECM is centrifuged at about 500 g for about 10 minutes, centrifuged at about 2,500 g for about 20 minutes, and centrifuged at about 10,000 g for about 30 minutes. These three steps are repeated three times. Thus, a fibril-free supernatant is produced.

The fibril-free supernatant is then centrifuged to isolate the MBV. In some embodiments, the fibril-free supernatant is centrifuged at about 100,000 g to about 150,000 g. Thus, the fibril-free supernatant is centrifuged at about 100,000 g to about 125,000 g, such as at about 100,000 g, about 105,000 g, about 110,000 g, about 115,000 g or about 120,000 g. This centrifugation can occur for about 60 to about 90 minutes, such as about 70 to about 80 minutes, for example for about 60, about 65, about 70, about 75, about 80, about 85 or about 90 minutes. In one non-limiting example, the fiber-free supernatant is centrifuged at about 100,000 g for about 70 minutes. The solid material is collected, which is the MBV. These MBV then can be re-suspended in any carrier of interest, such as, but not limited to, a buffer.

In further embodiments the ECM is not digested with an enzyme. In these methods, ECM is suspended in an isotonic saline solution, such as phosphate buffered saline. Salt is then added to the suspension so that the final concentration of the salt is greater than about 0.1 M. The concentration can be, for example, up to about 3 M, for example, about 0.1 M salt to about 3 M, or about 0.1 M to about 2M. The salt can be, for example, about 0.1M, 0.15M, 0.2M, 0.3M, 0.4 M, 0.7 M, 0.6 M, 0.7 M, 0.8M, 0.9M, 1.0 M, 1.1 M, 1.2 M, 1.3 M, 1.4 M, 1.5M, 1.6 M, 1.7 M, 1.8M, 1.9 M, or 2M. In some non-limiting examples, the salt is potassium chloride, sodium chloride or magnesium chloride. In other embodiments, the salt is sodium chloride, magnesium chloride, sodium iodide, sodium thiocyanate, a sodium salt, a lithium salt, a cesium salt or a calcium salt.

In some embodiments, the ECM is suspended in the salt solution for about 10 minutes to about 2 hours, such as about 15 minutes to about 1 hour, about 30 minutes to about 1 hour, or about 45 minutes to about 1 hour. The ECM can be suspended in the salt solution for about 15, 20, 25, 30, 35, 40, 45, 50, 55, 60, 65, 70, 75, 80, 85, 90, 95, 100, 105, 110, 115 or 120 minutes. The ECM can be suspended in the salt solution at temperatures from 4° C. to about 50° C., such as, but not limited to about 4° C. to about 25° C. or about 4° C. to about 37° C. In a specific non-limiting example, the ECM is suspended in the salt solution at about 4° C. In other specific non-limiting examples, the ECM is suspended in the salt solution at about 22° C. or about 25° C. (room temperature). In further non-limiting examples, the ECM is suspended in the salt solution at about 37° C.

In some embodiments, the method includes incubating an extracellular matrix at a salt concentration of greater than about 0.4 M; centrifuging the digested extracellular matrix to remove collagen fibril remnants, and isolating the supernatant; centrifuging the supernatant to isolate the solid materials; and suspending the solid materials in a carrier, thereby isolating MBV from the extracellular matrix.

Following incubation in the salt solution, the ECM is centrifuged to remove collagen fibrils. In some embodiments, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes. The supernatant is then collected.

In additional embodiments, the supernatant then can be centrifuged for a third step at about 100,000 to about 150,000 g. Thus, the digested ECM can be centrifuged at about 100,000 g to about 125,000 g, such as at about 100,000 g, 110,000 g or 120,000 g. This centrifugation can occur for about 30 minutes to about 2.5 hour, such as for about 1 hour to about 3 hours, for example for about 30 minutes, about 45 minutes, about 60 minutes, about 90 minutes, or about 120 minutes (2 hours). The solid materials are collected and suspended in a solution, such as buffered saline, thereby isolating the MBV.

In yet other embodiments, the ECM is suspended in an isotonic buffered salt solution, such as, but not limited to, phosphate buffered saline. Centrifugation or other methods can be used to remove large particles (see below). Ultrafiltration is then utilized to isolate MBV from the ECM, particles between about 10 nm and about 10,000 nm, such as between about 10 and about 1,000 nm, such as between about 10 nm and about 300 nm.

In specific non-limiting examples, the isotonic buffered saline solution has a total salt concentration of about 0.164 mM, and a pH of about 7.2 to about 7.4. In some embodiments, the isotonic buffered saline solution includes 0.002 M KCl to about 0.164 M KCL, such as about 0.0027 M KCl (the concentration of KCL in phosphate buffered saline). This suspension is then processed by ultracentrifugation.

Following incubation in the isotonic buffered salt solution, the ECM is centrifuged to remove collagen fibrils. In some embodiments, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes.

Microfiltration and centrifugation can be used and combined to remove large molecular weight materials from the suspension. In one embodiment, large size molecule materials, such as more than 200 nm are removed using microfiltration. In another embodiment, large size materials are removed by the use of centrifugation. In a third embodiment both microfiltration and ultracentrifugation are used to remove large molecular weight materials. Large molecular weight materials are removed from the suspended ECM, such as materials greater than about 10,000 nm, greater than about 1,000 nm, greater than about 500 nm, or greater than about 300 nm.

The effluent for microfiltration or the supernatant is then subjected to ultrafiltration. Thus, the effluent, which includes particle of less than about 10,000 nm, less than about 1,000 nm, less than about 500 nm, or less than about 300 nm is collected and utilized. This effluent is then subjected to ultrafiltration with a membrane with a molecular weight cutoff (MWCO) of 3,000 to 100,000. 100,000MWCO was used in the example.

### Methods for Treating Autoimmune Disorders

Methods are also disclosed herein for treating an autoimmune disorder (such as rheumatoid arthritis, scleroderma, ulcerative colitis, pemphigus, pemphigoid, Crohn's disease, psoriasis, psoriatic arthritis, multiple sclerosis, and/or systemic lupus erythematosus, among others) in a subject in need thereof. These methods include selecting a subject in need of treatment (such as in a subject with an autoimmune disorder, such as an rheumatoid arthritis, scleroderma, ulcerative colitis, pemphigus, pemphigoid, Crohn's disease, psoriasis, psoriatic arthritis, multiple sclerosis, and/or systemic lupus erythematosus) and administering to the subject a therapeutically effective amount of MBV (such as by administering a pharmaceutical preparation that includes a therapeutically effective amount of MBV) to decrease the autoimmune response, thereby treating the autoimmune disorder. In some examples, the MBV can be administered systemically. In specific examples, the MBV are administered by IV administration. In other examples, the autoimmune disease is rheumatoid arthritis. In other embodiments, the MBV are administered locally. In specific non-limiting examples, the autoimmune disease is psoriasis.

The subject can be a veterinary subject or a human. In a non-limiting example, the subject is a human. The subject can be a mammal. The subject can be avian or a domestic pet, such as a cat, dog or rabbit. The subject can be a non-human, primate (such as simians), or livestock, including swine, ruminants, horses, and poultry. The methods include selecting a subject in need of treatment to decrease an autoimmune response and administering to the subject a therapeutically effective amount of MBV (such as by administering a pharmaceutical preparation that includes a therapeutically effective amount of MBV). In some embodiments, the MBV can be administered systemically. In other embodiments, the MBV can be administered locally. The MBV can be derived from the same or a different species than the subject in need of decreased autoimmune activity. The MBV can be autologous.

The methods disclosed herein can result in a decrease in autoimmune activity in a subject. In some examples, signs or symptoms of the autoimmune disorder are reduced or eliminated. For example, the methods herein can result in complete or partial remission of the autoimmune disorder in a subject. In some examples, the methods herein can be used to reduce or eliminate flare-ups or relapse of the autoimmune disorder in a subject. In some examples, the methods herein can be used to reduce or eliminate the frequency or intensity of flare-ups of the autoimmune disorder in a subject. In some embodiments, the methods herein can be used to prevent progression of an autoimmune disorder in a subject.

Administration of the MBV (or pharmaceutical preparation that includes the MBV) can reduce or eliminate signs or symptoms of the autoimmune disorder in a subject for an extended period of time. In some embodiments, the subject experiences a therapeutic effect from a treatment course of MBV that lasts for a prolonged period of time. Preferably, the MBV are administered systemically. For example, this prolonged period of time may be a period of time beginning at the start of a course of treatment and ending 1 month, 2 months, 3 months, 4 months, 5 months, or 6 months thereafter. For example, this prolonged period of time may be a period of time beginning at the end of a course of treatment and ending 1 month, 2 months, 3 months, 4 months, 5 months, or 6 months thereafter. A therapeutic effect experienced by the subject may be (i) a reduction in the severity of the symptoms of the autoimmune disorder for the period of time as compared to the severity of the symptoms prior to the treatment course, (ii) remission of the autoimmune disorder or its symptoms for the period of time, (iii) prevention of flare up or relapse of the autoimmune disease during the period of time, (iv) reduction in the severity of symptoms experienced during a flare-up or relapse during the period of time compared to the severity of symptoms experienced prior to the treatment course, (v) reduction in the frequency of relapse or flare-up during the period of time as compared to frequency of relapse or flare up prior to the treatment course, or (vi) absence of signs of disease progression during the period of time after completing the treatment course. For any given autoimmune disorder, a reduction or improvement in the severity of symptoms or remission of the disorder can be measured according to relevant clinical indicia for a particular disorder and relevant clinical objective standards, for example, a scoring system for a particular disease or disorder. For example, a patient may experience a reduction in a clinical score for a disease or disorder during the time period indicative of improvement of the disorder as a result of the treatment course with MBV as compared to the score prior to the treatment course. The patient may experience a reduction in an autoimmune disorder score during the period of time as compared to the score prior to treatment and the score remains below the score prior to treatment during the period of time.

A subject may be administered 1 or more administrations of MBV constituting a course of treatment. A course of treatment is preferably administered systemically. A course of treatment may be administration of MBV 1 time per week for 4 weeks, 1 time per week for 3 weeks, 1 time per week for 2 weeks, 1 time per week for 1 week (i.e., only 1 administration), 2 times per week for 4 weeks, 2 times per week for 3 weeks, 2 times per week for 2 weeks, 2 times per week for 1 week, 3 times per week for 4 weeks, 3 times per week for 3 weeks, 3 times per week for two weeks, 3 times per week for 1 week, 4 times per week for 1 week, 4 times per week for two weeks, four times per week for three weeks, or 4 times per week for four weeks.

In one embodiment, a subject receives an initial treatment course 1 time per week for 4 weeks, 1 time per week for 3 weeks, 1 time per week for 2 weeks, 1 time per week for 1 week (i.e., only 1 administration), 2 times per week for 4 weeks, 2 times per week for 3 weeks, 2 times per week for 2 weeks, 2 times per week for 1 week, 3 times per week for 4 weeks, 3 times per week for 3 weeks, 3 times per week for two weeks, 3 times per week for 1 week, 4 times per week for 1 week, 4 times per week for two weeks, four times per week for three weeks, or 4 times per week for four weeks, followed by a maintenance course after 1 month, after 2 months, after 3 months, after 4 months, after 5 months, after 6 months, after 7 months, after 8 months, after 9 months, after 10 months, after 11 months, or after 12 months from completion of the initial treatment course. In one embodiment, the patient receives a maintenance course 6 months after the treatment course. The maintenance course may be the same or different than the treatment course. A maintenance course may be administration of MBV 1 time per week for 4 weeks, 1 time per week for 3 weeks, 1 time per week for 2 weeks, 1 time per week for 1 week (i.e., only 1 administration), 2 times per week for 4 weeks, 2 times per week for 3 weeks, 2 times per week for 2 weeks, 2 times per week for 1 week, 3 times per week for 4 weeks, 3 times per week for 3 weeks, 3 times per week for 2 weeks, 3 times per week for 1 week, 4 times per week for 1 week, 4 times per week for 2 weeks, 4 times per week for 3 weeks, or 4 times per week for 4 weeks. A maintenance course may be administered every 3 months, every 6 months, every 9 months, or every year. In one embodiment, a maintenance course is administered every 6 months.

According to some embodiments, a subject is administered 1×10⁶ to 1×10¹² MBV per kg of body weight per administration. In another embodiment, a subject is administered 1×10⁷ to 1×10¹¹ MBV per kg of body weight per administration. In another embodiment, a subject is administered 1×10⁷ to 1×10⁸ MBV per kg of body weight per administration. In another embodiment, a subject is administered 1×10⁸ to 1×10¹⁰ MBV per kg of body weight per administration. In another embodiment, a subject is administered 1×10⁹ to 1×10¹⁰ MBV per kg of body weight per administration. In another embodiment, a subject is administered 1×10⁶ to 1×10⁸ MBV per kg of body weight per administration. In another embodiment, a subject is administered 1×10⁷ to 1×10⁹ MBV per kg of body weight per administration. In another embodiment, a subject is administered 1×10⁸ to 1×10¹¹ MBV per kg of body weight per administration. In another embodiment, a subject is administered 1×10⁹ to 1×10¹¹ MBV per kg of body weight per administration. In another embodiment, a subject is administered 1×10⁶ to 1×10¹⁴ MBV per kg of body weight per administration. In another embodiment, a subject is administered 1×10¹² to 1×10¹⁴ MBV per kg of body weight per administration. In one embodiment, administration of MBV according to any of the aforementioned amounts is by systemic administration.

According to one embodiment, a subject is administered a course of treatment with MBV when the subject experiences a flare-up of symptoms associated with an autoimmune disorder. According to one embodiment, a subject is administered a course of treatment when the subject experiences progression of the autoimmune disease or disorder after remission. According to one embodiment, a subject is administered a course of treatment with MBV to prevent a flare-up or relapse of the autoimmune disease or disorder. For example, to prevent a flare-up or relapse, the subject is administered a course of treatment every 3 months, every 4 months, every 5 months, every 6 months, every 7 months, every 8 months, every 9 months, every 10 months, every 11 months, or once per year.

A variety of autoimmune disorders are included (such as a chronic autoimmune disorder). In some embodiments, the autoimmune disorder affects or primarily affects the skin, the respiratory system, the reproductive system, the cardiovascular system, or the nervous system. In some embodiments, the subject can have Addison's disease, alopecia areata, ankylosing spondylitis, anti-phospholipid antibody syndrome, autoimmune hepatitis, Celiac disease, Crohn's disease, Goodpasture's Syndrome, Grave's disease, Guillain-Barre syndrome, Hashimoto's thyroiditis, immune thrombocytopenia, IgA Nephropathy, inflammatory bowel disease (IBD), multiple sclerosis, myasthenia gravis, pemphigoid, pemphigus, polyglandular autoimmune syndrome type 2, psoriasis, psoriatic arthritis, rheumatoid arthritis, scleroderma, Sjogren's syndrome, systemic lupus erythematosus, Takayasu's arteriosus, type 1 diabetes, ulcerative colitis, or undifferentiated connective tissue disease (UCTD).

In some examples, the autoimmune disorder is a non-ocular autoimmune disorder. In some embodiments, the autoimmune disorder affects or primarily affects the skin, the respiratory system, the reproductive system, the cardiovascular system, or the nervous system. In some embodiments, the subject can have Addison's disease, alopecia areata, ankylosing spondylitis, anti-phospholipid antibody syndrome, autoimmune hepatitis, Celiac disease, Crohn's disease, Goodpasture's Syndrome, Grave's disease, Guillain-Barre syndrome, Hashimoto's thyroiditis, immune thrombocytopenia, IgA Nephropathy, multiple sclerosis, myasthenia gravis, pemphigoid, pemphigus, polyglandular autoimmune syndrome type 2, psoriasis, psoriatic arthritis, Sjogren's syndrome, systemic lupus erythematosus, Takayasu's arteriosus, type 1 diabetes, or undifferentiated connective tissue disease (UCTD). In some examples, the autoimmune disorder is not rheumatoid arthritis, scleroderma, or ulcerative colitis.

In some embodiments, the subject has rheumatoid arthritis (RA). In some examples, the methods include administering a therapeutically effective amount of MBV (such as by administering a pharmaceutical preparation that includes a therapeutically effective amount of MBV) to a subject having RA, thereby treating the RA. Preferably, the MBV are administered systemically, for example, by intravenous administration. The MBV may also be administered systemically, for example, by intraperitoneal, intramuscular, oral, enteral, parenteral, intranasal, rectal, sublingual, buccal, subcutaneous, or sublabial administration. The methods can include selecting a subject with RA. A variety of techniques can be used to identify RA in a subject. For example, RA can be identified using imaging (such as to identify synovial fluid from a joint, bone erosions, osteopenia near the joint, soft tissue swelling, and abnormally small joint space, subluxation, for example using X-ray, MRI, or ultrasound), blood tests (such as to identify rheumatoid factor (RF), anti-citrullinated protein antibodies (ACPAs, for example, as measured by anti-CCP antibodies, such as using an ELISA), erythrocyte sedimentation rate (ESR), C-reactive protein, full blood count, kidney function, liver enzyme levels, or antinuclear antibody/ANA), and the 2010 ACR/EULAR Rheumatoid Arthritis Classification Criteria (Aletaha et al., 2010 rheumatoid arthritis classification criteria: an American College of Rheumatology/European League Against Rheumatism collaborative initiative, Annals of the Rheumatic Diseases, 69 (9): 1580-8 (2010), incorporated herein by reference in its entirety). In some embodiments, the MBV are administered systemically to treat the RA. In specific examples, the MBV are administered by IV administration. In other examples, the MBV are administered by oral administration. In other embodiments, the MBV are administered by intramuscular, subcutaneous, or intraperitoneal administration. In some embodiments, the methods can decrease the severity or frequency of flare-ups of RA. For example, in some embodiments, the subject experiences a therapeutic effect in treating RA from a treatment course of MBV that lasts for a prolonged period of time. Preferably, the MBV are administered systemically. For example, this prolonged period of time may be a period of time of 1 month, 2 months, 3 months, 4 months, 5 months, or 6 months from the beginning of a course of treatment. For example, this prolonged period of time may be a period of time of 1 month, 2 months, 3 months, 4 months, 5 months, or 6 months from the end of a course of treatment. A therapeutic effect experienced by the subject may be (i) a reduction in the severity of the symptoms of RA for the period of time as compared to the severity of the symptoms prior to the treatment course, (ii) remission of the RA or its symptoms for the period of time, (iii) prevention of an RA flare-up or relapse during the period of time, (iv) reduction in the severity of symptoms experienced during an RA flare-up or relapse during the period of time compared to the severity of symptoms experienced prior to the treatment course, (v) reduction in the frequency of RA relapse or flare-up during the period of time as compared to frequency of relapse or flare-up prior to the treatment course, or (vi) absence of signs of RA disease progression during the period of time after completing the treatment course. For RA, a reduction or improvement in the severity of symptoms or remission of the disorder can be measured according to relevant clinical indicia and relevant clinical objective standards, for example, a scoring system such as the DAS28 for RA. For example, in one embodiment, the subject experiences a decreased arthritis score over a period of 1 month, two months, or three months from the start of a course of treatment as compared to the subject's arthritis score prior to the course of treatment, where the decreased score is maintained beyond the end of the course of treatment. In some embodiments, administration of MBV leads to remission of RA within 1 month, 2 months, 3 months, 4 months, 5 months, or 6 months from the time of beginning a course of treatment, wherein the remission is maintained beyond the end of the course of treatment. In some embodiments, administration of MBV leads to a decrease in a subject's DAS28 score below 5.1 during a period of time from the beginning of a course of treatment and ending 1 month, 2 months, 3 months, 4 months, 5 months, or 6 months thereafter In some embodiments, administration of a course of MBV treatment leads to a decrease in a subjects DAS28 score below 3.2 (low disease activity) during a period of time from the beginning of a course of treatment and ending 1 month, 2 months, 3 months, 4 months, 5 months, or 6 months thereafter. In some embodiments, administration of MBV leads to a decrease in a subjects DAS28 score below 2.6, i.e., the administration achieves remission during a period of time from the beginning of a course of treatment and ending 1 month, 2 months, 3 months, 4 months, 5 months, or 6 months thereafter. In one embodiment, the subject experiences a decrease in the DAS28 score during a period of time measured from the beginning of a course of treatment and ending 1 month, 2 months, 3 months, 4 months, 5 months, or 6 months thereafter. In some embodiments, the decrease in the DAS28 score is maintained beyond the end of the course of treatment for a period of 1 month, 2 months, 3 months, 4 months, 5 months, 6 months or more. In one embodiment, the decrease in DAS28 score is achieved via systemic administration of MBV. In one embodiment, the therapeutic effect in treating RA extends beyond the duration of the treatment course, for example, by one month, two months, three months, four months, five months, six months or more.

In some embodiments, the subject has scleroderma. In some examples, the methods include administering a therapeutically effective amount of MBV (such as by administering a pharmaceutical preparation that includes a therapeutically effective amount of MBV), thereby treating the scleroderma. Preferably, the MBV are administered systemically, for example, by intravenous administration. A variety of scleroderma types can be treated using the disclosed methods, such as localized scleroderma, for example, localized morphea, morphea-lichen sclerosus et atrophicus overlap (LSA), generalized morphea, atrophoderma of pasini and pierini, pansclerotic morphea, morphea profunda, linear scleroderma, and systemic scleroderma, for example, CREST syndrome and progressive systemic sclerosis. In some embodiments, the MBV are administered systemically to treat the scleroderma. In specific examples, the MBV are administered by IV or oral administration. The MBV may also be administered by intraperitoneal, subcutaneous, or intramuscular administration. The methods can decrease the severity or frequency of flare-ups of scleroderma. The methods can also lead to remission of scleroderma.

The methods can include selecting a subject with scleroderma. A variety of techniques can be used to identify a subject with scleroderma. For example, testing for scleroderma can include clinical diagnosis (such as identifying areas of thickened skin, stiffness, fatigue, and poor blood flow to the fingers or toes upon cold exposure), blood tests (such as for elevated levels of immune factors, as known as antinuclear antibodies), pulmonary function tests (such as to measure lung function, for example, using an X-ray or a computed tomography (CT scan), such as to identify lung damage), electrocardiogram (such as to identify congestive heart failure or defective electrical activity of the heart), echocardiogram (such as to identify pulmonary hypertension or congestive heart failure), gastrointestinal tests (such as an endoscopy or manometry), or kidney tests (such as using blood tests to identify high levels of protein). In some embodiments, the MBV are administered systemically to treat the scleroderma. In specific examples, the MBV are administered by IV administration. In other examples, the MBV are administered by oral administration. In other embodiments, the MBV are administered locally, such as to the skin. The methods of administration can decrease the frequency of severity of flare-ups of scleroderma. The methods can also lead to remission of scleroderma. For example, the original or modified Rodnan score can be used to measure the severity of scleroderma. The Rodnan score is determined by scoring the severity of skin thickening of the 17 anatomic surface areas of the body from 0 to 3 and summing all surface scores. A surface score of 3 represents severely thick skin with the inability to pinch the skin into a fold. A surface score of 0 is healthy for adults, while children can have healthy scores of 0 or 1. In one embodiment, the subject experiences a reduction in a surface score to 0 or 1 as a result of MBV administration and experiences remission. In another embodiment, a subject experiences a reduction in surface score to 2 or less (3 is indicative of severe disease) as a result of MBV administration. In one embodiment, the subject experiences a decrease in Rodnan score from the time of MBV administration that remains decreased over 1 month, two months, or three months from administration In one embodiment, this decrease is achieved via systemic administration of MBV. In one embodiment, the therapeutic effect in treating scleroderma extends beyond the duration of the treatment course, for example, by one month, two months, three months, four months, five months, six months or more.

In some embodiments the subject has ulcerative colitis. In some examples, the methods include administering a therapeutically effective amount of MBV (such as by administering a pharmaceutical preparation that includes a therapeutically effective amount of MBV), thereby treating the ulcerative colitis. Preferably, the MBV are administered systemically, for example, by intravenous administration. The methods can include selecting a subject with ulcerative colitis. A variety of techniques can be used to identify a subject ulcerative colitis. For example, testing for ulcerative colitis can include a complete blood count (such as to identify anemia or thrombocytosis), electrolyte or kidney function tests (such as to identify hypokalemia, hypomagnesemia, or pre-renal failure, liver function tests (such as to identify primary sclerosing cholangitis), X-ray, urinalysis, stool culture (such as to identify parasites or infectious agents), erythrocyte sedimentation rate or C-reactive protein measurement (such as to identify inflammation), or sigmoidoscopy (such as to identify ulcers in the large intestine. In some examples, the clinical colitis activity index can be used to assess the severity of the ulcerative colitis. In some embodiments, the MBV are administered systemically to treat the ulcerative colitis. In specific examples, the MBV are administered by IV administration. In other examples, the MBV are administered by oral administration. In some examples, the MBV are administered by intraperitoneal, subcutaneous, or intramuscular administration. The MBV can be administered locally, such as to the gut. The methods can decrease the severity or frequency of flare-ups of ulcerative colitis. The methods can also lead to clinical remission and endoscopic remission of ulcerative colitis disease. For example, in some embodiments, administration of the MBV results in a decrease in the Mayo Score or Ulcerative Colitis Disease Activity Index (UCDAI) for ulcerative colitis as compared to the score prior to treatment. In one embodiment, a patient experiences a reduction in Mayo score to 2 or less and experiences remission. In another embodiment, a patient experiences a reduction in Mayo score to 5 or less. In another embodiment, a patient experiences a reduction in Mayo score to less than 10. In one embodiment, the subject experiences a decrease in the Mayo score or UCDAI score measured from the time of beginning a course of MBV treatment that remains decreased over 1 month, 2 months, or 3 months thereafter. In one embodiment, this decrease is achieved via systemic administration of MBV. In one embodiment, the therapeutic effect in treating ulcerative colitis extends beyond the duration of the treatment course, for example, by one month, two months, three months, four months, five months, six months or more.

In some embodiments, the subject has Crohn's disease. In some examples, the methods include administering a therapeutically effective amount of MBV (such as by administering a pharmaceutical preparation that includes a therapeutically effective amount of MBV), thereby treating the Crohn's disease. Preferably, the MBV are administered systemically, for example, by intravenous administration. In some examples, the MBV are administered by intraperitoneal, subcutaneous, or intramuscular administration. A variety of types of Crohn's disease can be treated using the disclosed methods, including ileocolic Crohn's, Crohn' colitis, Gastroduodenal Crohn's, and Jejunoileitis. Crohn's disease can be treated that is caused by an agent, such as Crohn's disease caused by immune system dysfunction (for example, autoimmunity or impaired innate immunity), genetic factors, changes in gut bacteria, and environmental factors. The methods can include selecting a subject with Crohn's disease. A variety of techniques can be used to identify a subject with Crohn's disease. For example, testing for Crohn's disease can include endoscopy (such as a colonoscopy), imaging (such as using a barium follow-through X-ray, CT scans, and MRI scans), and blood tests (such as to identify an iron, a vitamin D, or a vitamin B12 deficiency; erythrocyte sedimentation rate (ESR); and C-reactive protein levels). In some embodiments, the MBV are administered systemically to treat the Crohn's disease. In specific examples, the MBV are administered by IV administration. In other examples, the MBV are administered by oral administration. The MBV can be administered locally, such as to the gut. The methods can decrease the severity or frequency of flare-ups of Crohn's disease. The methods can also lead to clinical remission and endoscopic remission of Crohn's disease. For example, in some embodiments, administration of the MBV results in a decrease in the Crohn's Disease Activity Index (CDAI) as compared to the score prior to treatment. In one embodiment, a patient experiences a reduction in CDAI score to below 150 and experiences remission. In another embodiment, a patient experiences a reduction in CDAI score to below 450 or less (450 or greater is indicative of severe disease). In another embodiment, a patient experiences a fall of at least 70 CDAI points (indicative of therapeutic response) as a result of MBV treatment. For example, In another embodiment, a patient experiences a fall of at least 70 CDAI points (indicative of therapeutic response) as a result of MBV treatment from the time of MBV administration that remains decreased over 1 month, 2 months, or 3 months or more from administration. In one embodiment, the subject experiences a decrease in the CDAI score from the time of MBV administration that remains decreased over 1 month, 2 months, or 3 months from administration. In one embodiment, this decrease is achieved via systemic administration of MBV. In one embodiment, the therapeutic effect in treating Crohn's disease extends beyond the duration of the treatment course, for example, by one month, two months, three months, four months, five months, six months or more.

In some embodiments, the subject has pemphigus. In some examples, the methods include administering a therapeutically effective amount of MBV (such as by administering a pharmaceutical preparation that includes a therapeutically effective amount of MBV), thereby treating the pemphigus. Preferably, the MBV are administered systemically, for example, by intravenous administration. In some examples, the MBV are administered by intraperitoneal, subcutaneous, or intramuscular administration. In some examples, the MBV are administered locally by topical cutaneous administration. A variety of pemphigus types can be treated using the disclosed methods, such as pemphigus vulgaris, pemphigus foliaceus, IgA pemphigus, or paraneoplastic pemphigus. The methods can include selecting for a subject with pemphigus. A variety of techniques can be used to identify a subject with pemphigus. For example, testing for pemphigus can include clinical diagnosis (such as by lesions at the eye and mucous membrane of the oral cavity), skin or mucous membrane biopsy (such as to identify intraepidermal vesicles caused by acantholysis), and an ELISA on a blood sample or a direct immunofluorescence on the skin biopsy (such as to identify anti-desmoglein autoantibodies). In some embodiments, the MBV are administered systemically to treat the pemphigus. In specific examples, the MBV are administered by IV administration. In other examples, the MBV are administered by oral administration. In other embodiments, the MBV are administered locally to the skin. The methods can decrease the severity or frequency of flare-ups of pemphigus. The methods can also lead to remission of pemphigus. A reduction in severity of symptoms can be based on a decrease in the PDAI (pemphigus Disease Index) or a decrease in the Autoimmune Bullous Skin Disorder Intensity Score (ABSIS) from the time of MBV administration. Moderate disease is PDAI<15 or ABSIS<17, significant disease is PDAI between 15 and 44 or ABSIS between 17 and 53, and extensive disease is PDAI greater than 45 or ABSIS greater than 53. In one embodiment, the subject experiences a reduction in PDAI score to below 15 or ABSIS score to below 17 and experiences remission. In another embodiment, the subject experiences a reduction in reduction in PDAI score to below 45 or ABSIS score to below 53 or less (PDAI score 45 or greater or ABSIS score 53 or greater is indicative of severe disease). In one embodiment, the subject experiences a decrease in the PDAI score from the time of MBV administration that remains decreased over 1 month, 2 months, or 3 months from administration. In one embodiment, this decrease is achieved via systemic administration of MBV. In one embodiment, the therapeutic effect in treating pemphigus extends beyond the duration of the treatment course, for example, by one month, two months, three months, four months, five months, six months or more.

In other embodiments, the subject has pemphigoid. In some examples, the methods include administering a therapeutically effective amount of MBV (such as by administering a pharmaceutical preparation that includes a therapeutically effective amount of MBV), thereby treating the pemphigoid. Preferably, the MBV are administered systemically, for example, by intravenous administration. In some examples, the MBV are administered by intraperitoneal, subcutaneous, or intramuscular administration. In some examples, the MBV are administered locally by topical cutaneous administration. A variety of pemphigoid types can be treated using the disclosed methods, such as IgG-mediated pemphigoid, for example, gestational, bullous, and cicatricial pemphigoid, as well as IgA-mediated pemphigoid, for example, IgA-mediated immunobullous diseases. The methods can include selecting a subject with pemphigoid. A variety of techniques can be used to select a subject that has pemphigoid. For example, testing for pemphigoid can include clinical diagnosis (such as to identify tense blisters and erosions on skin without another identifiable cause; desquamative gingivitis or mucositis involving oral, ocular, nasal, genital, anal, pharyngeal, laryngeal, and/or esophageal mucosae; or unexplained pruritus, pruritic eczematous eruptions, or urticarial plaques), histopathology (such as to identify lesional tissue, for example, using punch biopsy with haemotoxylin and eosin (H&E) staining), direct immunofluorescence (DIF; such as to identify tissue-bound antibodies in biopsy specimens), indirect immunofluorescence (such as to identify circulating antibodies targeting the antigens at the basement membrane zone, for example, using a saliva sample), and antigen-specific serologic testing (such as to identify autoantibodies against NC16A, BP180, BP230, laminin 332, or type VII collagen, for example using an ELISA. In some embodiments, the MBV are administered systemically to treat the pemphigoid. In specific examples, the MBV are administered by IV administration. In other examples, the MBV are administered by oral administration. In other embodiments, the MBV are administered locally to the skin. The methods can decrease the severity of frequency of flare-ups of pemphigoid. The methods can also lead to remission of pemphigoid. A reduction in severity of symptoms can be based on a decrease in the PDAI (pemphigus Disease Index) or a decrease in the Autoimmune Bullous Skin Disorder Intensity Score (ABSIS) from the time of MBV administration. Moderate disease is PDAI<15 or ABSIS<17, significant disease is PDAI between 15 and 44 or ABSIS between 17 and 53, and extensive disease is PDAI greater than 45 or ABSIS greater than 53. In one embodiment, the subject experiences a reduction in PDAI score to below 15 or ABSIS score to below 17 and experiences remission. In another embodiment, the subject experiences a reduction in reduction in PDAI score to below 45 or ABSIS score to below 53 or less (PDAI score 45 or greater or ABSIS score 53 or greater is indicative of severe disease). In one embodiment, the subject experiences a decrease in the PDAI or ABSIS score from the time of MBV administration that remains decreased over 1 month, 2 months, or 3 months from administration. In one embodiment, this decrease is achieved via systemic administration of MBV. In one embodiment, the therapeutic effect in treating pemphigoid disease extends beyond the duration of the treatment course, for example, by one month, two months, three months, four months, five months, six months or more.

In further embodiments, the subject has psoriasis. In some examples, the methods include administering a therapeutically effective amount of MBV (such as by administering a pharmaceutical preparation that includes a therapeutically effective amount of MBV), thereby treating the psoriasis. In some embodiments, methods are disclosed for treating psoriasis in a subject in need thereof that include administering to the subject a pharmaceutical preparation comprising a therapeutically effective amount of isolated MBV derived from extracellular matrix, thereby treating the psoriasis in the subject. The pharmaceutical preparation can be administered systemically. The pharmaceutical composition can be administered locally, such as topically tothe skin of the subject. In some embodiments, the pharmaceutical composition is administered to one or more plaques on the skin of the subject. Various types of psoriasis are included, such as plaque, guttate, inverse, pustular, and erythrodermic psoriasis. The methods can include selecting a subject that has psoriasis. A variety of techniques can be used to identify psoriasis in a subject. For example, testing for psoriasis can include clinical diagnosis (such as by identifying scaly, erythematous plaques, papules, or patches of skin that may be painful and itch) or a skin biopsy or scraping (such as to identify clubbed epidermal projections that interdigitate with dermis, epidermal thickening, abnormal skin cells from the most superficial skin layer, or inflammatory infiltrates). In some embodiments, the MBV are administered systemically to treat the psoriasis. In specific examples, the MBV are administered by IV administration. In other examples, the MBV are administered by oral administration. In other embodiments, the MBV are administered locally to skin or lesions. The methods can decrease the frequency or severity of flare-ups of psoriasis. The methods can lead to remission of psoriasis. A reduction in severity of symptoms or remission can be based on a decrease in the PASI score (Psoriasis Area and Severity Index), wherein multiple criteria are scored between 0 and 4, and the criteria are summed. Mild psoriasis is a PASI score between 0 and 5, moderate psoriasis is a PASI score between 5 and 12, severe psoriasis is a PASI score between 12 and 20, and very severe psoriasis is a PASI score greater than 20. In one embodiment, the subject experiences PASI75, an improvement of 75% or greater in PASI score from baseline, indicative of therapeutic response, as a result of a course of MBV treatment. In one embodiment, the subject experiences a reduction in PASI score to about 0 and experiences remission as a result of a course of MBV treatment. In another embodiment, the subject experiences a reduction in PASI score to below 20 or less (20 or greater is indicative of severe disease) as a result of a course of MBV treatment. In one embodiment, a subject experience a decrease in PASI score from the time of beginning a course of MBV treatment that remains decreased over 1 month, 2 months, or 3 months or more from administration. In one embodiment, this decrease is achieved via systemic administration of MBV. In one embodiment, the therapeutic effect in treating psoriasis extends beyond the duration of the treatment course, for example, by one month, two months, three months, four months, five months, six months or more.

In more embodiments, the subject has psoriatic arthritis. In some examples, the methods include administering a therapeutically effective amount of MBV (such as by administering a pharmaceutical preparation that includes a therapeutically effective amount of MBV), thereby treating the psoriatic arthritis. Preferably, the administration of MBV is by Various types of psoriatic arthritis can be treated using the disclosed methods such as oligoarticular, polyarticular, arthritis mutilans, arthritis mutilans, spondyloarthritis, and distal interphalangeal predominant. The methods can include selecting for a subject with psoriatic arthritis. A variety of techniques can be used to identify a subject with psoriatic arthritis. For example, testing for psoriatic arthritis can include clinical diagnosis (such as to identify family history of psoriasis or psoriatic arthritis, onycholysi, distal Interphalangeal articulations of hand, enthesitis, or dactylitis) blood tests (such as to identify a negative result for rheumatoid factor), and X-ray (such as to identify degenerative joint changes). In some embodiments, the MBV are administered systemically to treat the psoriatic arthritis. In specific examples, the MBV are administered by IV administration. In other examples, the MBV are administered by or oral administration. In other embodiments, the MBV are administered locally, such as intra-articularly. The methods can decrease the frequency or severity of flare-ups of psoriatic arthritis. The methods can lead to remission of psoriatic arthritis. A decrease in Disease Activity Index for Psoriatic Arthritis (DAPSA) score can be used to determine response. In one embodiment, the subject experiences a reduction in DAPSA score to below 4 and experiences remission as a result of a course of MBV treatment. In some embodiments, the subject experiences a reduction in DAPSA score of 50%, 75%, or 85%, indicative of therapeutic response as a result of a course of MBV treatment. In another embodiment, the subject experiences a reduction in DAPSA score to below 28 (28 or higher is indicative of high Disease Activity) as a result of a course of MBV treatment. In one embodiment, the subject experiences a decrease in the DAPSA score as a result of MBV administration that remains decreased over 1 month, 2 months, or 3 months from beginning a course of MBV treatment. In one embodiment, this decrease is achieved via systemic administration of MBV. In one embodiment, the therapeutic effect in treating psoriatic arthritis extends beyond the duration of the treatment course, for example, by one month, two months, three months, four months, five months, six months or more.

In some embodiments, the subject has multiple sclerosis. In some examples, the methods include administering a therapeutically effective amount of MBV (such as by administering a pharmaceutical preparation that includes a therapeutically effective amount of MBV), thereby treating the multiple sclerosis. Preferably, the MBV are delivered by systemic administration, for example, intravenous administration. In some examples, the MBV are administered by intraperitoneal, subcutaneous, or intramuscular administration. A variety of multiple sclerosis types can be treated using the disclosed methods, such as relapsing-remitting, secondary-progressive, primary progressive, and clinically isolated syndrome multiple sclerosis. The methods can include selecting a subject with multiple sclerosis. A variety of techniques can be used to identify a subject with multiple sclerosis. For example, testing for multiple sclerosis can include clinical diagnosis (such as by physical, mental, and psychiatric symptoms, for example, double vision, blindness in one eye, muscle weakness, trouble with sensation, or coordination), imaging (such as to identify areas of demyelination, for example, by lesions or plaques), lumbar puncture (such as to identify oligoclonal bands of IgG, for example, in cerebrospinal fluid, such as by electrophoresis), visual- and sensory-evoked potentials, and biopsy. In some embodiments, the MBV are administered systemically to treat the multiple sclerosis. In specific examples, the MBV are administered by IV administration. In other embodiments, the MBV are administered by oral administration. The methods can decrease the frequency or severity of flare-ups of multiple sclerosis. The methods can lead to remission of multiple sclerosis. The methods can also prevent disease progression. A decrease in Expanded Disability Status Score (EDSS) can be used to determine response. In one embodiment, a patient experiences a reduction in EDSS score to 1.5 or below, indicative of disability remission, as a result of a course of MBV treatment. In another embodiment, a subject's ability to walk 25 feet improves, as a result of a course of MBV treatment. In another embodiment, a patient experiences a reduction in EDSS score to below 8.5 or less (8.5 or greater is indicative of very severe disability). In one embodiment, the subject experiences a decrease in the EDSS score as a result of MBV administration that remains decreased over 1 month, 2 months, 3 months, 4 months, 5 months, or 6 months from beginning a course of MBV treatment. In one embodiment, this decrease is achieved via systemic administration of MBV. In one embodiment, the therapeutic effect in treating MS extends beyond the duration of the treatment course, for example, by one month, two months, three months, four months, five months, six months or more.

In more embodiments, the subject has systemic lupus erythematosus (SLE). In some examples, the methods include administering a therapeutically effective amount of MBV (such as by administering a pharmaceutical preparation that includes a therapeutically effective amount of MBV), thereby treating the SLE. Preferably, the MBV are administered by systemic administration, for example, by intravenous administration. In some examples, the MBV are administered by intraperitoneal, subcutaneous, or intramuscular administration. Systemic lupus erythematosus due to a variety of causes can be treated using the disclosed methods, such as SLE due to genetic factors, drug reactions, or other lupus (such as discoid, cutaneous lupus). The methods can include selecting a subject with SLE. A variety of techniques can be used to select subjects with SLE. For example, testing for SLE can include serologic tests (such as for antinuclear antibody (ANA), anti-extractable nuclear antigen (anti-ENA), anti-dsDNA, anti-U1 RNP (which also appears in systemic sclerosis and mixed connective tissue disease), SS-A (or anti-Ro), and SS-B (or anti-La), complement system levels, electrolyte levels and kidney function, liver enzyme levels, complete blood count, and the lupus erythematosus (LE) cell test. In some embodiments, the MBV are administered systemically to treat the SLE. In specific examples, the MBV are administered by IV administration. In other embodiments, the MBV are administered by oral administration. The methods can decrease the severity or frequency of flare-ups of SLE. The methods can lead to remission of SLE. Disease severity can be measured by the SLE Disease Activity Index (SLEDAI) or BILAG score. In one embodiment, the subject experiences a reduction in SLEDAI score to 3 or below or a BILAG score to D or E, indicative of remission as a result of a course of MBV treatment. In one embodiment, the subject experiences a reduction in SLEDAI score of 4 or more or a reduction of BILAG score to C or below, indicative of therapeutic response, as a result of a course of MBV treatment. In another embodiment, a subject experiences a reduction in SLEDAI score to below 7.5 or less or reduction of BILAG score from A to B (a SLEDAI score of 7.5 or greater or a BILAG score of A is indicative of severe disease), as a result of a course of MBV treatment. In one embodiment, a subject experiences a decrease in the SLEDAI or BILAG score as a result of MBV administration that remains decreased over 1 month, 2 months, 3 months, 4 months, 5 months, or 6 months from the time of beginning a course of MBV treatment. In one embodiment, this decrease is achieved via systemic administration of MBV. In one embodiment, the therapeutic effect in treating MS extends beyond the duration of the treatment course, for example, by one month, two months, three months, four months, five months, six months or more.

The methods can include selecting for a subject with autoimmune encephalitis. In some examples, the methods include administering a therapeutically effective amount of MBV (such as by administering a pharmaceutical preparation that includes a therapeutically effective amount of MBV), thereby treating the autoimmune encephalitis. A variety of techniques can be used to select subjects with autoimmune encephalitis. For example, selecting subjects with encephalitis can include a brain scan (such as using MRI) to determine inflammation; EEG (such as monitoring brain activity, in which encephalitis will produce abnormal signal), lumbar puncture (spinal tap), blood test, urine analysis, and polymerase chain reaction (PCR) testing of the cerebrospinal fluid to detect the presence of viral DNA (such as to identify viral encephalitis). In some embodiments, the MBV are administered systemically to treat the autoimmune encephalitis. In specific examples, the MBV are administered by IV or oral administration. In some embodiments, the MBV are administered by systemic administration, for example, by intravenous administration. In some examples, the MBV are administered by intraperitoneal, subcutaneous, or intramuscular administration. In other embodiments, the MBV are administered locally to the brain, such as by intracerebral injection. In some embodiments, the methods of administration can decrease the frequency or severity of flare-ups of autoimmune encephalitis or can decrease inflammation associated with autoimmune encephalitis such that a patient is no longer has autoimmune encephalitis or the autoimmune encephalitis is in remission. In one embodiment, the therapeutic effect in treating autoimmune encephalitis extends beyond the duration of the treatment course, for example, by one month, two months, three months, four months, five months, six months or more.

Administration can be systemic. Exemplary routes of systemic administration include, but are not limited to, intravenous administration, oral administration, enteral administration, parenteral administration, intranasal administration, rectal administration, sublingual administration, buccal administration, sublabial administration, intraperitoneal administration, transdermal, transmucosal, subcutaneous, or intramuscular administration. In specific, non-limiting examples, the systemic administration is intravenous administration.

Administration may be local. Exemplary routes of local administration include intraarticular injection, topical cutaneous administration, intrathecal administration and intradermal administration, or by direct injection or application onto or into a tissue or organ of interest.

Dosage treatment may be a single dose schedule or a multiple dose schedule to ultimately deliver 1×10⁶ to 1×10¹² MBV (i.e., an absolute number of vesicles) per kg body weight per administration. Administration may be provided as a single administration, a periodic bolus or as continuous infusion, such as by continuous release for a specific period from a sustained-release drug or drug delivery device. The subject may be administered as many doses as appropriate. If multiple doses are administered, administration can be intermittent. In example embodiments, administration (such as systemic administration, for example, intravenous administration, or any other route of administration) of a therapeutically effective amount of MBV can be performed once, or can be performed repeatedly, such as 1, 2, 3, 4, 5, 6, 7, 8, 9, or 10 times. In example embodiments, administration can be performed biweekly, weekly, every other week, monthly, or every 2, 3, 4, 5, or 6 months. In other embodiments, only a single administration is required to achieve therapeutic benefit. In other embodiments, only one course of treatment is required to achieve therapeutic benefit for 1 month, 2 months, 3 months, 4 months, 5 months, or 6 months from beginning the course of treatment. In other embodiments, only one course of treatment is required to achieve therapeutic benefit lasting 1 month, 2 months, 3 months, 4 months, 5 months, or 6 months from ending the course of treatment.

Individual doses are typically not less than an amount required to produce a measurable effect on the subject, and may be determined based on the pharmacokinetics and pharmacology for absorption, distribution, metabolism, and excretion (“ADME”) of the subject composition or its by-products and, thus, based on the disposition of the composition within the subject. This includes consideration of the route of administration as well as dosage amount, which can be adjusted for local and systemic (for example, intravenous) applications. Effective amounts of dose and/or dose regimen can readily be determined empirically from preclinical assays, from safety and escalation and dose range trials, individual clinician-patient relationships, as well as in vitro and in vivo assays. Generally, these assays will evaluate the autoimmune disorder (such as rheumatoid arthritis, scleroderma, ulcerative colitis, pemphigus, Crohn's disease, psoriasis, psoriatic arthritis, sclerosis, or systemic lupus erythematosus).

A therapeutically effective amount of MBV can be suspended in a pharmaceutically acceptable carrier (such as in a pharmaceutical preparation), for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. Other agents can be added to the compositions, such as preservatives and anti-bacterial agents. These compositions can be administered locally or systemically, such as intravenously.

Pharmaceutical preparations that include a therapeutically effective amount of MBV can be formulated in unit dosage form, suitable for individual administration of precise dosages. The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated. In example embodiments, treatment with the MBV results in a decrease or a reduction in a sign or symptom of an autoimmune disorder (such as rheumatoid arthritis, scleroderma, ulcerative colitis, pemphigus, Crohn's disease, psoriasis, psoriatic arthritis, sclerosis, or systemic lupus erythematosus) present at the time of administration.

Administration of MBV results in a therapeutic benefit to a subject. The therapeutic benefit can vary, for example, as a function of time and/or intensity. In example embodiments, the subject experiences a therapeutic benefit for prolonged period of time after administration of the MBV. For example, the subject can experience a therapeutic benefit lasting a time period of at least 3 days, at least 1 week, at least 2 weeks, at least 3 weeks, at least 4 weeks, at least 1 month, at least 2 months, at least 3 months, at least 4 months, at least 5 months, at least 6 months, or more.

In some embodiments, the subject experiences a therapeutic benefit from the administration of MBV lasting a time period of at least 2 weeks, at least 3 weeks, at least 4 weeks, at least 1 month, at least 2 months, or at least 3 months. In more embodiments, the therapeutic benefit from the administration lasts at least one week beyond completion of a course of treatment. In yet other embodiments, the therapeutic benefit from the administration last at least two weeks beyond completion of a course of treatment. In further embodiments, the therapeutic benefit from the administration lasts at least one month beyond completion of a course of treatment. In some embodiments, the therapeutic benefit from the administration lasts at least two months beyond completion of a course of treatment. In further embodiments, the therapeutic benefit from the administration last at least three months or more beyond completion of a course of treatment. In further embodiments, the therapeutic benefit from the administration last at least six months or more beyond completion of a course of treatment. In yet other embodiments, the therapeutic benefit is a reduction in a symptom of the disorder present at the time of administration.

In some examples, treatment with the MBV can result in a decrease or a reduction in autoimmune activity in a subject over the level of autoimmune activity prior to administration of the MBV. In some examples, treatment with MBV can result in remission of the autoimmune disorder. In some examples, treatment with MBV can result in a reduction or elimination of flares of signs or symptom of an autoimmune disorder (such as rheumatoid arthritis, scleroderma, ulcerative colitis, pemphigus, Crohn's disease, psoriasis, psoriatic arthritis, sclerosis, or systemic lupus erythematosus). For example, treatment with MBV can result in a reduction or elimination of flares or signs or symptoms of an autoimmune disorder for a prolonged time period beyond completion of a course of treatment (such as of at least 3 days, at least 1 week, at least 2 weeks, at least 3 weeks, at least 4 weeks, at least 1 month, at least 2 months, at least 3 months, or at least 6 months). The treatment may result in remission of the disease or disorder.

The subject can be administered additional therapeutic agents, in the same or different composition or pharmaceutical preparation. In example embodiments, the subject has an autoimmune disorder (such as rheumatoid arthritis, scleroderma, ulcerative colitis, pemphigus, Crohn's disease, psoriasis, psoriatic arthritis, sclerosis, or systemic lupus erythematosus), and the subject is administered additional therapeutic agents, such as anti-inflammatories and/or immunosuppressing drugs can be administered.

In some embodiments, the method includes the step of detecting that a therapeutic benefit has been achieved. Measures of therapeutic efficacy will be applicable to the particular disease being modified, and a person of skill in the art will recognize the appropriate detection methods to use to measure therapeutic efficacy. The subject can be evaluated for response using any methods known in the art. In example embodiments, the subject has an autoimmune disorder (such as rheumatoid arthritis, scleroderma, ulcerative colitis, pemphigus, Crohn's disease, psoriasis, psoriatic arthritis, sclerosis, or systemic lupus erythematosus), and the therapeutic response in a subject can be measured by an antinuclear antibody test (ANA) or specific autoantibodies produced in certain autoimmune types, an examination for inflammation in the body, and the like.

Exemplary methods are disclosed below.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Differentiating Matrix Bound Vesicles (MBV) and Extracellular Vesicles (EV) Through Lipidomics and RNA Sequencing

Matrix-bound nanovesicles (MBV) have been reported as an integral component of ECM bioscaffolds. Although liquid-phase extracellular vesicles (EV) have been the subject of intense investigation, their similarity to MBV is limited to size and shape. This example utilized LC-MS-based lipidomics and redox lipidomics to conduct a detailed comparison of liquid-phase EV and MBV phospholipids. Combined with comprehensive RNA sequencing and bioinformatic analysis of the intravesicular cargo, this example shows that MBV are a distinct and unique subpopulation of EV, and a distinguishing feature of ECM-based biomaterials.

This example identifies similarities and differences between liquid phase (i.e., exosomes) and matrix bound forms (i.e., MBV) of EV. However, given that EV present in biological fluids and MBV present in native tissue ECM and ECM-based biomaterials represent heterologous populations secreted from multiple cell sources, a direct comparative in-vivo analysis between these putative EV populations is problematic. As an alternative to using body fluid or tissue-derived vesicles, ECM and conditioned media produced in-vitro by cultured cells can be isolated (Fitzpatrick et al., Biomater Sci., 3, 12-24 (2015). This approach offers several advantages such as the use of a single cell type source thereby obviating any doubts regarding vesicle origin; the ability to selectively harvest vesicles from either liquid or solid phase compartments; and the ability to control the cell culture environment and thus also control vesicle composition and cargo.

### Materials and Methods

Preparation of in vitro cell-derived ECM: Human bone marrow stem cells (BMSC), human adipose stem cells (ASC) and human umbilical cord stem cells (UCSC) ECM plates were provided by StemBioSys (San Antonio, Texas) and prepared according to a published protocol (Lai et al., Stem cells and development 19, 1095-1107 (2010)). Briefly, human BMSC, human ASC, or human UCSC were seeded onto a 75 cm²-cell culture flask coated with human fibronectin (1 h at 37° C.) at a cell density of 3,500 cells/cm² and cultured in α-MEM medium supplemented with 20% fetal bovine serum (FBS) and 1% penicillin-streptomycin for 14 days. The medium was refreshed the day after initial seeding and then every 3 days. At day 7, ascorbic acid 2-phosphate (Sigma Aldrich) was added to the medium at a final concentration of 50 μM. At day 14, plates were decellularized using 0.5% Triton in 20 mM ammonium hydroxide for 5 min, rinsed two times with Hank's Balanced Salt Solution containing both calcium and magnesium (HBSS+/+), and once with ultra-pure H₂O. Murine NIH 3T3 fibroblast cells were seeded onto a 75 cm²-cell culture flask at a cell density of 3,500 cells/cm² and cultured in DMEM medium supplemented with exosome-depleted FBS (G. V. Shelke, et al, Journal of extracellular vesicles 3, 24783 (2014)), 1% penicillin-streptomycin and ascorbic acid 2-phosphate (Sigma Aldrich) at a final concentration of 50 μM for 7 days. At day 7, the supernatant from cultured 3T3 fibroblast cells was collected, and the plated culture was washed 3 times with PBS, decellularized using 0.5% Triton in 20 mM ammonium hydroxide for 5 min, and then rinsed three times with ultra-pure H₂O.

Isolation of MBV and liquid-phase EV: MBV were isolated (L. Huleihel et al., Science advances 2, e1600502 (2016)). Briefly, the decellularized ECM was enzymatically digested with 100 ng/ml Liberase DL (Roche) in buffer (50 mM Tris pH 7.5, 5 mM CaCl₂, 150 mM NaCl) for 1 hr at 37° C. The cell culture supernatant containing the liquid-phase EV and the digested ECM containing the MBV were subjected to differential centrifugation at 500 g (10 min), 2500 g (20 min), and 10,000 g (30 min), and the supernatant passed through a 0.22 μm filter (Millipore). The clarified supernatant containing the liberated MBV or liquid-phase EV was then centrifuged at 100,000×g (Beckman Coulter Optima L-90K Ultracentrifuge) at 4° C. for 70 min to pellet the vesicles. The vesicle pellets were then washed and resuspended in 1×PBS, and stored at −20° C. until further use.

Preparation of urinary bladder matrix (UBM): UBM was prepared from market-weight pigs (Tissue Source; LLC, Lafayette, IN) (L. Huleihel et al., Science advances 2, e1600502 (2016)). Briefly, the tunica serosa, muscularis externa, submucosa, and muscularis mucosa were removed by mechanical delamination, and the urothelial cells of the tunica mucosa were dissociated from the basement membrane by washing with deionized water. The remaining basement membrane and the lamina propria (collectively referred to as UBM) were decellularized by agitation in 0.1% peracetic acid with 4% ethanol for 2 h at 300 rpm followed by phosphate-buffered saline (PBS) and type 1 water washes. UBM was then lyophilized and milled using a Wiley Mill with a #60 mesh screen.

Scanning Electron Microscopy (SEM): UBM was fixed in cold 2.5% glutaraldehyde for 24 hours followed by three 30 minute washes in 1×PBS. Samples were then dehydrated in a graded alcohol series (30%, 50%, 70%, 90%, 100% ethanol) for 30 minutes per wash, and then placed in 100% ethanol overnight at 4° C. Samples were washed 3 additional times in 100% ethanol for 30 minutes each and critical point dried using a Leica EM CPD030 Critical Point Dryer (Leica Microsystems, Buffalo Grove, IL, USA) with carbon dioxide as the transitional medium. Samples were then sputter-coated with a 4.5 nm thick gold/palladium alloy coating using a Sputter Coater 108 Auto (Cressington Scientific Instruments, UK) and imaged with a JEOL JSM6330f scanning electron microscope (JEOL, Peabody, MA, USA)

Transmission Electron Microscopy (TEM): TEM imaging was conducted on MBV or liquid-phase EV loaded on carbon-coated grids and fixed in 4% paraformaldehyde (L. Huleihel et al., Science advances 2, e1600502 (2016)). Grids were imaged at 80 kV with a JEOL 1210 TEM with a high-resolution Advanced Microscopy Techniques digital camera. Size of the MBV was determined from representative images using JEOL TEM software.

Nanoparticle Tracking Analysis (NTA): Particle size and concentration of the liquid-phase EV and the MBV were calculated using a Nanosight (NS300) instrument equipped with fast video capture and particle-tracking software. Samples were diluted 1:500 to a final volume of 1000 μl using particle-free water. A syringe pump was used to dispense the sample into the system. Measurements were performed from three captures of 45 seconds each sample. For the video processing and particle calculation, the detection threshold was adjusted to 4. Data is presented as concentration vs. particle size for each of the evaluated samples.

RNA isolation: Total RNA was isolated from 3T3 cells, liquid-phase EV and MBV using the RNeasy mini kit (Qiagen) according to the manufacturer's instructions. Before RNA isolation, liquid-phase EV and MBV samples were treated with RNase A (10 μg/ml) at 37° C. for 30 min to degrade any contaminating RNA. RNA quantity was determined using NanoDrop spectrophotometer, and its quality was determined by Agilent Bioanalyzer 2100 (Agilent Technologies).

RNA sequencing and bioinformatic analysis: The miRNA library preparation was initiated with 100 ng of each sample, and the QIAseq™ miRNA Library Kit (Qiagen) following manufacturer's instructions. Briefly, mature miRNAs were ligated to adapters on their 3′ and 5′ ends. The ligated miRNAs were then reverse transcribed to cDNA using a reverse transcription (RT) primer with a unique molecular indices (UMI). The cDNA was then cleaned up to remove adapter primers, followed by amplification of the library with a universal forward primer and one of 48 reverse primers that assigns a sample index. A pre-sequencing quality control was performed using the Agilent RNA ScreenTape System. Next Generation Sequencing was performed on a NextSeq 500 instrument with a loading concentration of 2.5 μM. Bioinformatic analysis was conducted by Genevia Technologies (Tampere, Finland). The quality of the sequencing reads was inspected using FastQC software. TrimGalore! [Version 0.4.5;] was used to remove the adapter sequences, with default settings, on all the samples. All reads were shortened to 21 bases, the typical size of micro-RNAs, using the fastx_trimmer software (FASTX Toolkit by Hannon Lab; Version 0.0.14). The reads of each sample were then aligned against the corresponding reference genome (hg38, GRCm38). Tables of miRNA counts across samples were created using the software bowtie [Version 1.2.2] and miRDeep2 [Version 0.0.8]. In this process, precursor-miRNA and mature-miRNA sequences for each species involved in the study were taken from miRbase. Counts of mature miRNAs were obtained by taking the median of all precursor miRNAs associated with them. The counts of mature miRNAs of all samples were normalized using DESeq2. To ensure data quality before further analyses, principal component analysis (PCA) was performed and the results were visualized using ggplot2, separately for murine and human samples. Normalization of mature miRNA data and statistical testing between sample groups was performed with DESeq2. P values were corrected for multiple testing using Benjamini-Hochberg method. miRNAs with adjusted p value<0.05 and absolute log 2 fold change>1 were considered as significantly differentially expressed. Tables of differentially expressed miRNAs were annotated with their targets and their confidences using the mirTARbase database of experimentally tested miRNA-target interactions. Differentially expressed miRNAs were also annotated with predicted targets using the R package miRNAtap. miRNAtap aggregates the miRNA target predictions from five different databases (PicTar, DIANA, TargetScan, miRanda, miRDB) and calculates an overall miRNA target score. The minimum amount of database sources required for a potential miRNAtarget interaction to be included into the annotations was 3.

Ingenuity pathway analysis (IPA): Ingenuity Pathway Analysis software (Version 01-14) was used for functional analysis of differentially expressed (DE) miRNAs. miRNA targets were identified using the IPA Core Analysis. The filter was set to Experimentally Observed findings to obtain information about significantly enriched molecular and cellular functions and physiological system development functions that were affected by the miRNAs.

qPCR validation: Reverse transcription (RT) and quantitative polymerase chain reaction (qPCR) were performed using the TaqMan® Advanced miRNA Assays Protocol (Applied Biosystems). Briefly, 10 ng total RNA were used with The TaqMan® Advanced miRNA cDNA Synthesis Kit (Applied Biosystems, Cat No. A28007) to synthetize and adapt a 3′-poly(A) tail to the miRNAs. Universal RT primers recognizing the poly(A) tail were used to synthetize the cDNA in the RT reaction, followed by a miR-AMP step, using miR-AMP forward and reverse universal primers, to increase the number of cDNA molecules. The qPCR was made on a QuantStudio™ system machine using the TaqMan® Fast Advanced Master Mix (Applied Biosystems, Cat No. 4444556) and specific TaqMan® Advanced miRNA Assays (Applied Biosystems, Cat No. A25576) recognizing mmu-miR-163-5p, mmu-miR-27a-5p, mmu-miR-92a-1-5p, mmu-miR-451a, mmu-miR-93-5p, and mmu-miR-99b-5p. Fold change expression on the MBV sample was calculated for each of the specific targets using Liquid-phase EV as a reference.

Immunoblot and silver stain assays: Liquid-phase EV and MBV, derived from three separate cultures of 3T3 fibroblasts, were respectively pooled and quantified by nanotracking particle analysis. For both immunoblot and silverstain analysis, an equal number of vesicles for both the liquid-phase EV and MBV samples were loaded onto the gel. 21×10¹¹ MBV or liquid-phase EV were mixed with 2× Laemmli buffer (R&D Systems) containing 5% R mercaptoethanol (Sigma-Aldrich), resolved on a 4 to 20% gradient SDS-PAGE (Bio-Rad), and then transferred onto a PVDF membrane. Membranes were incubated overnight with the following primary antibodies: rabbit anti-CD63, rabbit anti-CD81, rabbit anti-CD9, and rabbit anti-Hsp70, at 1:1000 dilution (System Biosciences). Membranes were washed three times for 15 min each before and after they were incubated with goat anti-rabbit secondary antibody, at 1:5,000 dilution (System Biosciences). The washed membranes were exposed to chemiluminescent substrate (Bio-Rad) and then visualized using a ChemiDoc Touch instrument (Bio-Rad). Silver staining of gels was performed using the Silver Stain Plus Kit (Bio-Rad) according to the manufacturer's instruction and visualized using a ChemiDoc Touch instrument (Bio-Rad).

LC/MS analysis of phospholipids: Lipids were extracted from 3T3 cells, exosomes and MBV by Folch procedure (J. Folch, et al, J biol Chem 226, 497-509 (1957)). MS analysis of phospholipids and their oxygenated products was performed on an Orbitrap™ Fusion™ Lumos™ mass spectrometer (ThermoFisher) (Y. Y. Tyurina et al., ACS nano 5, 7342-7353 (2011)). Briefly, phospholipids were separated on a normal phase column (Luna 3 μm Silica (2) 100Å, 150×2.0 mm, (Phenomenex)) at a flow rate of 0.2 ml/min on a Dionex Ultimate 3000 HPLC system. The column was maintained at 35° C. The analysis was performed using gradient solvents (A and B) containing 10 mM ammonium acetate. Solvent A contained propanol:hexane:water (285:215:5, v/v/v) and solvent B contained propanol:hexane:water (285:215:40, v/v/v). All solvents were LC/MS grade. The column was eluted for 0-23 min with a linear gradient from 10% to 32% B; 23-32 min using a linear gradient of 32-65% B; 32-35 min with a linear gradient of 65-100% B; 35-62 min held at 100% B; 62-64 min with a linear gradient from 100% to 10% B followed by and equilibration from 64 to 80 min at 10% B. Spectra were acquired in negative ion mode. Deuterated phospholipids were used as internal standards (Avanti Polar Lipids). Three technical replicates for each sample were run to evaluate reproducibility. Analysis of LC/MS data was performed using software package Compound Discoverer™ (ThermoFisher) with an in-house generated analysis workflow and non-oxidized/oxidized phospholipid database. Lipids were further filtered by retention time and confirmed by fragmentation mass spectrum.

LC/MS Analysis of free fatty acids and their oxidation products: Free fatty acids were analyzed by LC/MS using a Dionex Ultimate™ 3000 HPLC system coupled on-line to Q-Exactive hybrid quadrupole-orbitrap mass spectrometer (ThermoFisher Scientific, San Jose, CA) (Y. Y. Tyurina et al., Nature chemistry 6, 542 (2014).). Briefly, fatty acids and their oxidative derivatives were separated by a C18 column (Accliam PepMap RSLC, 300 μm 15 cm, Thermo Scientific) using a gradient of solvents (A: Methanol (20%)/Water (80%) (v/v) and B: Methanol (90%)/Water (10%) (v/v) both containing 5 mM ammonium acetate. The column was eluted at a flow rate of 12 μL/min using a linear gradient from 30% solvent B to 95% solvent B over 70 min, held at 95% B from 70 to 80 min followed by a return to initial conditions by 83 min and re-equilibration for an additional 7 min. Spectra were acquired in negative ion mode. Analytical data were acquired and analyzed using Xcalibur software. A minimum of three technical replicates for each sample was run to increase the reproducibility.

### Results

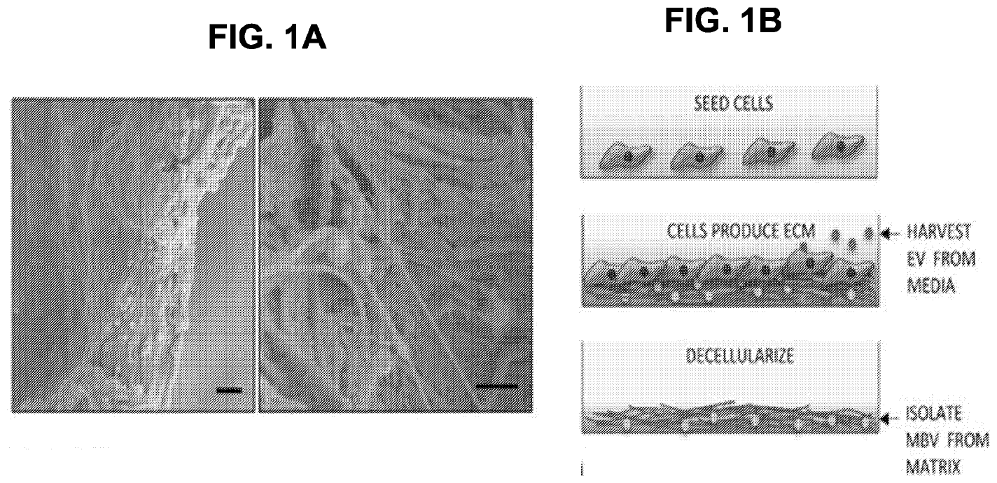

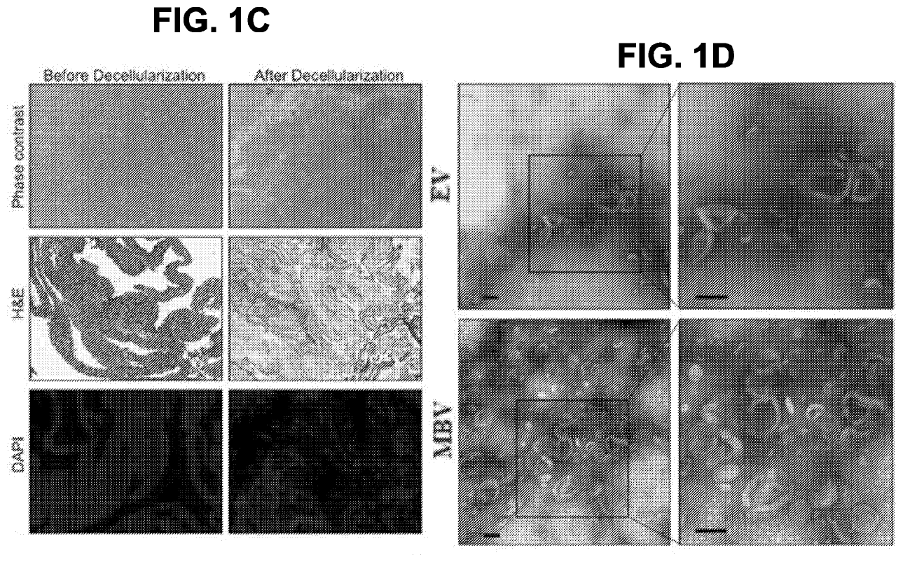

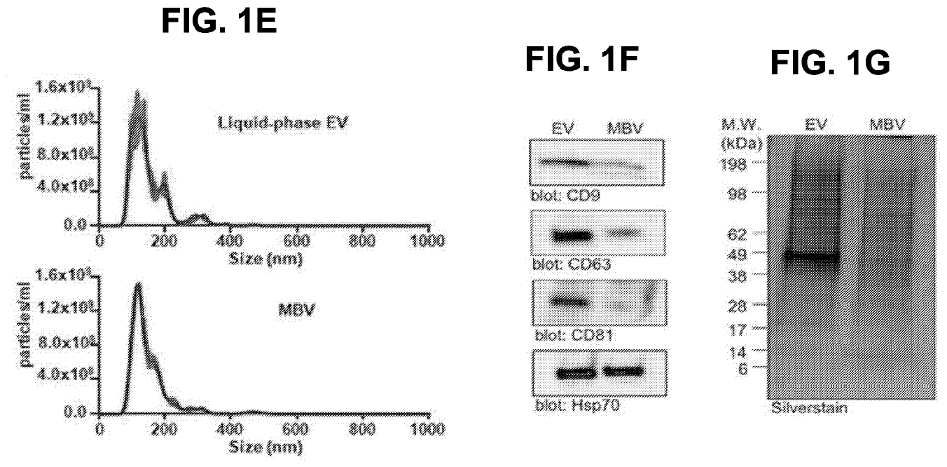

Isolation of liquid-phase EV and matrix bound nanovesicles: Scanning electron microscopy (SEM) was performed to provide high-resolution, high-magnification imaging of MBV embedded within an ECM bioscaffold derived from porcine urinary bladder matrix (UBM). SEM images revealed discrete spheres approximately 100 nm in diameter dispersed throughout the collagen fibers (FIG. 1A). To examine if MBV deposited into a solid ECM substrate are a unique class of extracellular vesicle separate from EV secreted into a liquid-phase, an in-vitro 3T3 fibroblast cell culture model that allows for selective harvesting of vesicles from a liquid-phase or solid-phase extracellular compartment was used (FIG. 1B). Representative images from phase contrast microscopy, and H&E and DAPI stained sections showed that no residual cells or intact nuclei were visible after decellularization of the cell culture plate (FIG. 1C). TEM imaging of liquid-phase EV harvested from the cell culture supernatant and MBV isolated from decellularized ECM (FIG. 1D) showed that these two populations of vesicles shared a similar morphology. Moreover, nanoparticle tracking analysis (NTA) distribution plots showed similar vesicle size of both liquid-phase EV and MBV, with the majority of vesicles having a diameter<200 nm (FIG. 1E). To determine whether MBV contained markers commonly attributed to exosomes, immunoblot analysis was performed for CD63, CD81, CD9, and Hsp70 (J. Lötvall et al. (Taylor & Francis, 2014)). Results showed that in contrast to liquid-phase EV, the MBV showed a marked decrease in CD63, CD81, CD9. MBV expressed levels of CD9 and CD81 that were barely detectable in the immunoblot assay and markedly decreased relative to the levels expressed in EV. MBV also displayed significantly lower expression of CD63 than was observed in EV. (FIG. 1F). In other words, liquid phase EV (i.e., exosomes) are enriched in expressed levels of CD63, CD81, CD9 as compared to MBV. Furthermore, silver staining of electrophoretically-separated proteins showed that MBV contained a protein cargo that was distinctly different than the liquid-phase EV (FIG. 1G), suggesting that MBV may be a unique subpopulation of nanovesicles.

miRNA is selectively packaged into liquid-phase EV and MBV derived from 3T3 fibroblasts: Comprehensive next generation RNA-sequencing (RNA-seq) was employed to catalog differentially expressed miRNA in MBV and liquid-phase EV relative to the 3T3 fibroblast parent cell from which these vesicles were derived. Bioanalyzer analysis revealed the absence of 18S and 28S ribosomal RNA, and an enrichment of small RNA molecules (<200 nt) in total RNA isolated from liquid-phase EV and MBV. However, the small RNA size distribution from liquid phase EV was much broader than MBV with a marked enrichment of small RNA molecules between 100-200 nt in liquid-phase EV (FIG. 2A). Analysis was focused on differential miRNA signatures by conducting next generation sequencing of miRNA libraries generated from the parental cellular RNA, the liquid-phase EV, and the MBV isolates (n=3 per group). Principal component analysis (PCA) showed that within respective groups, the replicate miRNA profiles clustered close to one another (FIG. 2B).

Extensive differences in miRNA content were observed between the parental cell and the liquid-phase EV and MBV isolates. Overall, 28 (50.91%) miRNAs were found to be differentially expressed in MBV compared to liquid-phase EV by at least two-fold (FIG. 2C). Additionally, respective liquid-phase EV or MBV and the parental cellular miRNA profiles were clearly distinct (FIG. 2B, FIG. 2C). To validate the results of miRNA sequencing, RT-qPCR was conducted to detect 3 upregulated miRNAs (miR-163-5p, miR-27a-5p, miR-92a-1-5p) and 3 downregulated miRNAs (miR-451a, miR-93b-5p, miR-99b-5p) in MBV compared to liquid phase EV isolated from 3T3 fibroblasts (FIG. 2D). The results showed that the levels of miR-163-5p, miR-27a-5p and miR-92a-1-5p were upregulated, and miR-451a, miR-93b-5p and miR-99b-5p were downregulated in MBV compared to liquid-phase EV, thereby corroborating the results from the miRNA sequencing data. Ingenuity Pathway Analysis (IPA) of differentially enriched miRNAs in MBV compared to liquid-phase EV showed a strong association with organ and system development and function. In contrast, miRNA differentially enriched in liquid-phase EV compared to MBV were associated with pathways involved in cellular growth, development, proliferation and morphology (FIG. 2E).

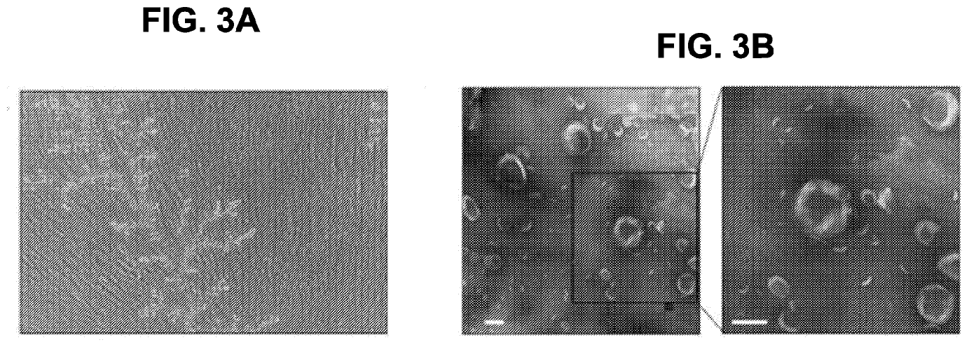

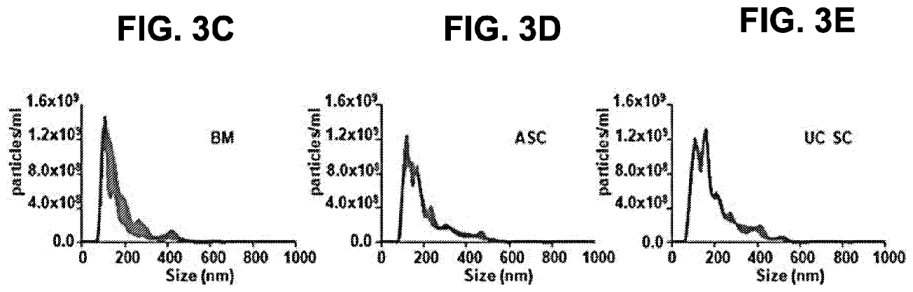

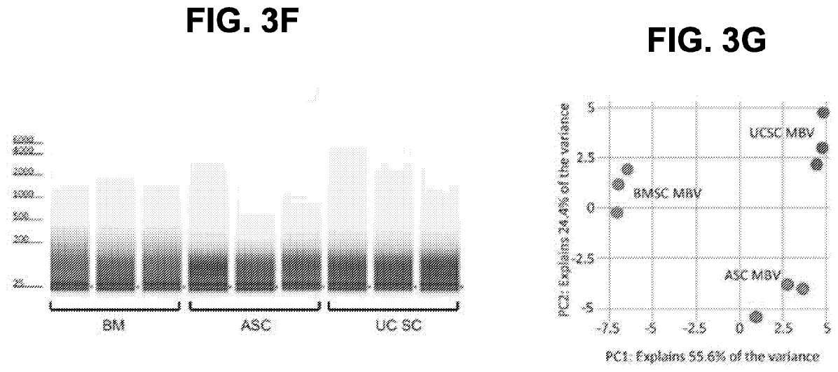

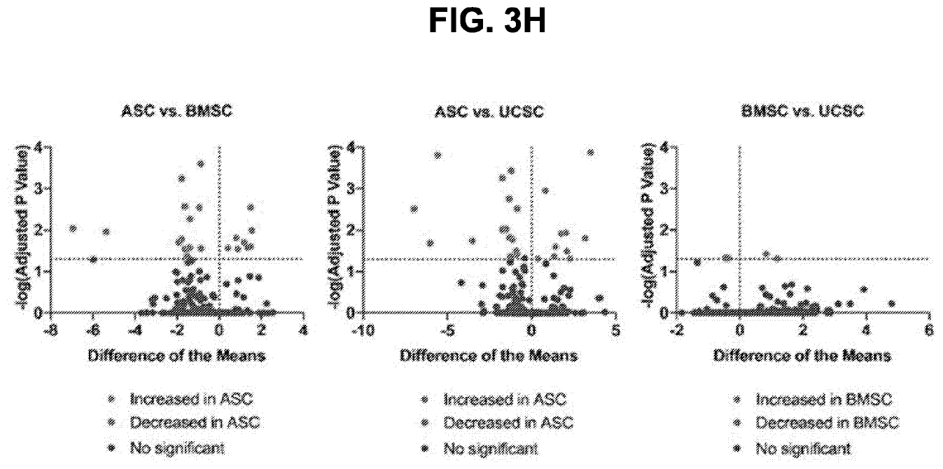

MBV miRNA content is unique to the cellular origin: Results with the 3T3 fibroblast cell model showed selective packaging of miRNA within MBV deposited in the ECM compared to liquid-phase EV secreted into the cell culture supernatant. To determine if MBV miRNA cargo is unique to the cellular origin, the miRNA composition of MBV isolated from ECM produced in-vitro by bone marrow-derived stem cells (BMSC), adipose stem cells (ASC) and umbilical cord stem cells (UCSC) isolated from different human donors, were characterized and compared through next generation sequencing methods. A representative phase contrast microscopy image of a decellularized BMSC cell culture plate showed the absence of cells and the presence of branched fibrillar structures (FIG. 3A). TEM imaging of isolated MBV from a decellularized BMSC cell culture plate showed the characteristic morphology attributed to extracellular vesicles (FIG. 3B). Furthermore, nanoparticle tracking analysis showed similar distribution plots between BMSC, ASC, and UCSC-derived MBV, with the majority of vesicles having a diameter<200 nm (FIGS. 3C-3E). After isolation of total RNA from these samples, bioanalyzer analysis showed the absence of ribosomal RNA and an enrichment of small RNA molecules (<200 nt) (FIG. 3F). miRNA libraries were generated from the samples (BMSC, n=3 human donors; ASC, n=3 human donors; UCSC, n=3 human donors) and subjected to miRNA sequencing. A principal component analysis showed that samples clustered primarily by the cell type from which they were derived (FIG. 3G). Despite the use of three separate human donors for each cell type used to generate the MBV samples, the principal component analysis showed a high degree of homogeneity in the miRNA profile within the respective groups (FIG. 3G). In addition, volcano plots showed that fewer miRNAs were found differentially expressed between BMSC and UCSC-derived MBV than between BMSC-ASC and UCSC-ASC.

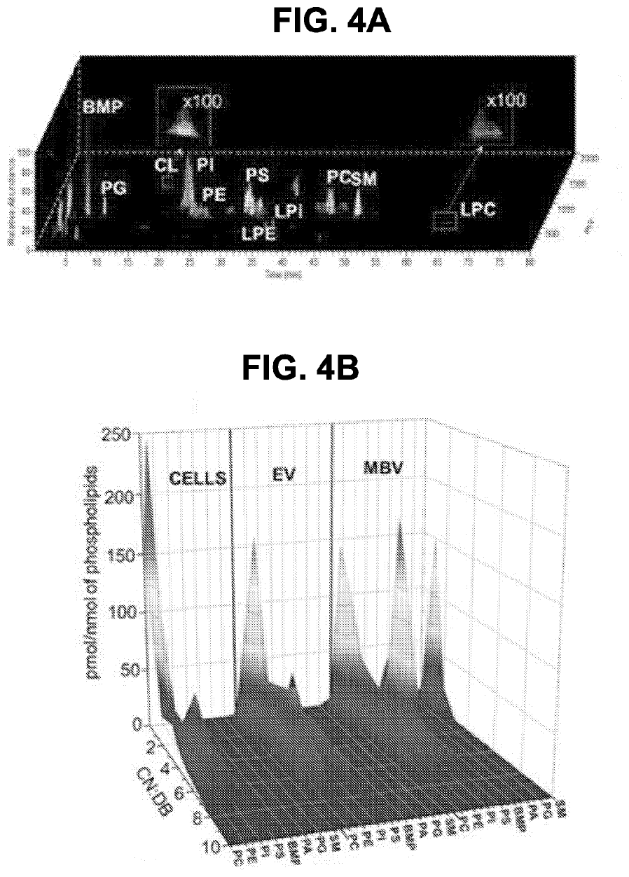

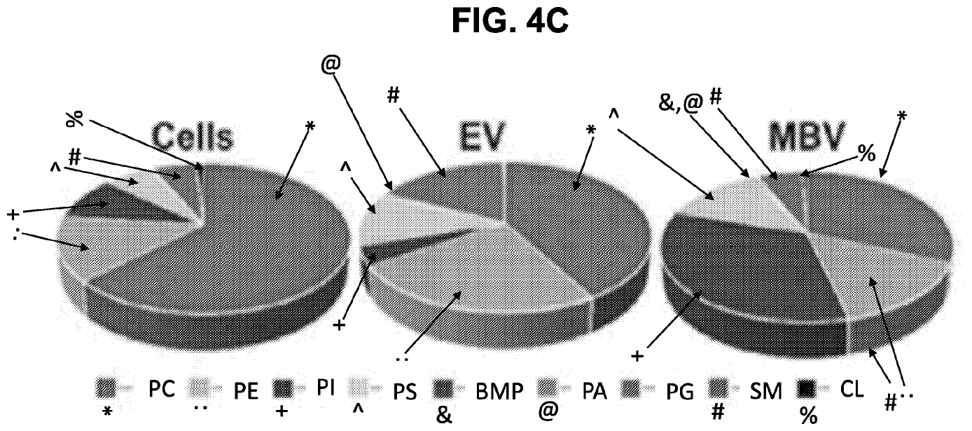

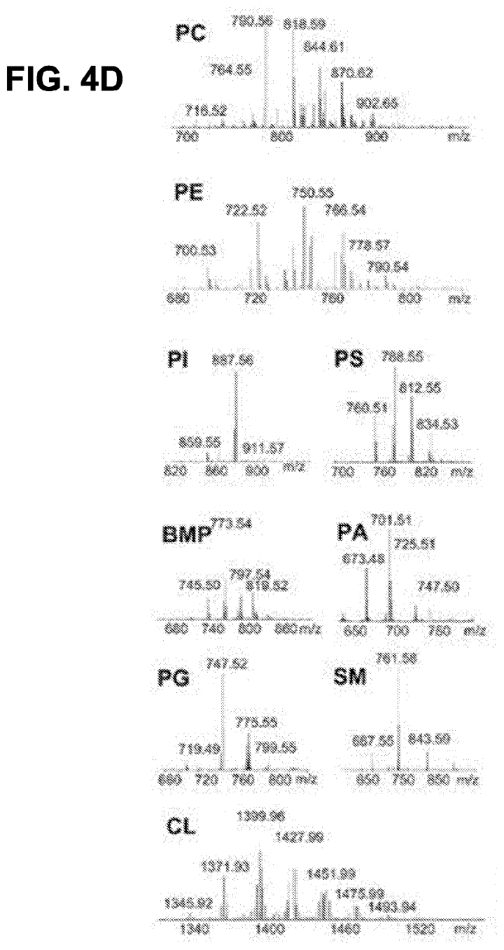

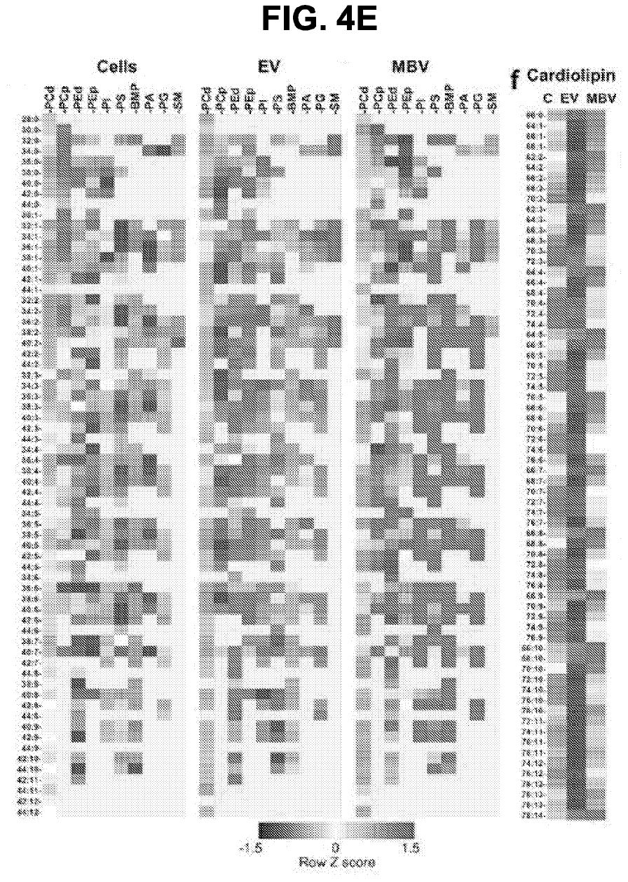

Phospholipid profiles of liquid-phase EV, MBV, and parent cells: Several studies have characterized the lipid composition of EV (T. Skotland, et al, Journal of lipid research 60, 9-18 (2019)). However, there is no data on phospholipid composition of MBV. LC-MS based global lipidomics and redox lipidomics analyses were therefore conducted to comparatively evaluate the phospholipid composition of MBV and liquid-phase EV compared to their 3T3 fibroblast parent cells (FIG. 4A, FIG. 4D). Nine major phospholipid classes were detected across the three types of samples, with the total number of detected molecular species of 536 distributed between the following major classes: bis-monoacylglycerophosphate (BMP)—59 species, phosphatidylglycerol (PG)—37 species, cardiolipin (CL)—117 species, phosphatidylinositol (PI)—33 species, phosphatidylethanolamine (PE)—102 species, phosphatidylserine (PS)—45 species, phosphatidic acid (PA)—26 species, phosphatidylcholine (PC)—107 species, and sphingomyelin (SM)—10 species (FIG. 4D). In terms of their content of polyunsaturated fatty acid (PUFA) residues, PE, PI, PC and PS represented the major reservoir of these polyunsaturated PL species containing four-seven double bonds (FIG. 4B). These PUFA phospholipids represent the likely precursors of the signaling lipid mediators. The formation of the mediators occurs via the catalytic oxygenation of PUFA phospholipids by 5-lipoxygenase or 15-lipoxygenase to yield oxygenated phospholipids that are subsequently hydrolyzed by one of specialized phospholipases A2 to release oxygenated fatty acids (lipid mediators) (Z. Zhao et al., Endocrinology 151, 3038-3048 (2010); Y. Y. Tyurina et al., Journal of leukocyte biology, (2019)). In addition, oxidized PUFA phospholipids act as signaling molecules coordinating many intracellular processes and cell responses, including apoptosis, ferroptosis and inflammation (Y. Y. Tyurina et al., Antioxidants & redox signaling 29, 1333-1358 (2018).). Significant differences in molecular speciation of these phospholipids and their relative contents were observed between liquid-phase EV and MBV (FIG. 4E). With a notable exception of SM, arachidonic acid (AA)- and docosahexaenoic acid (DHA)-residues were detected in all phospholipids (FIG. 4E). For many of the phospholipids, the amounts were significantly higher in MBV vs liquid-phase EV and parent cells (FIG. 4E), which identify MBV as a rich reservoir of PUFA-phospholipids. PUFA phospholipids can be hydrolyzed by PLA₂ resulting in the release of free PUFA and LPL (V. D. Mouchlis, et al, Biochimica et Biophysica Acta (BBA)-Molecular and Cell Biology of Lipids 1864, 766-771 (2019)). The former can be further utilized by two major oxygenases, COX and LOX to produce lipid mediators with pro- or anti-inflammatory capacities (Y. Y. Tyurina et al., Redox (phospho) lipidomics of signaling in inflammation and programmed cell death. Journal of leukocyte biology, (2019); C. A. Rouzer, et al., Chemical reviews 103, 2239-2304 (2003).; H. Kuhn, et al., Biochimica et Biophysica Acta (BBA)-Molecular and Cell Biology of Lipids 1851, 308-330 (2015)). This finding qualifies MBV as potential precursors for synthesis of these lipid mediators dependently on the cell/tissue context (Y. Y. Tyurina et al., Journal of leukocyte biology, (2019).). Quantitatively, MBV were enriched in PI, PS, PG and BMP (FIG. 4C and Table 2). The phospholipid content shown in FIG. 4C is also provided in Table 1.

TABLE 1
Phospholipid Content as Percent of Total Phospholipids

| | Cells | EV | MBV |
|---|---|---|---|
| PC, phosphatidylcholine | 62.95 | 41.54 | 31.38 |
| PE, phosphatidylethanolamine | 13.84 | 24.47 | 14.75 |
| PI, phosphatidylinositol | 9.6 | 4.21 | 33.58 |
| PS, phosphatidylserine | 6.49 | 11.18 | 12.68 |
| BMP, bis-monoacylglycerophosphate | 0.13 | 0.13 | 0.55 |
| PA, phosphatidic acid | 0.11 | 0.64 | 0.42 |
| PG, phosphatidylglycerol | 0.04 | 0.02 | 0.11 |
| SM, sphingomyelin | 5.87 | 17.64 | 5.87 |
| CL, cardiolipin | 0.97 | 0.17 | 0.66 |

In contrast, the content of PE, PA and SM was higher in liquid-phase EV. PC was a predominant phospholipid in cells and liquid-phase EV. The content of a unique mitochondrial phospholipid, cardiolipin (CL), was significantly lower in liquid-phase EV compared to MBV and parent cells (FIG. 4F). Because CL is a unique mitochondria-specific phospholipid localized predominantly in the inner mitochondrial membrane (M. Schlame, et al., Biochimica et Biophysica Acta (BBA)-Molecular and Cell Biology of Lipids 1862, 3-7 (2017)), this finding represents a possible link of the MBV biogenesis with the mitochondrial compartment of cells. Plasmalogen phospholipids (or ether phospholipids) are structurally different from diacyl-phospholipids (or ester-phospholipids) (M. Schlame, et al., Biochimica et Biophysica Acta (BBA)-Molecular and Cell Biology of Lipids 1862, 3-7 (2017)). In plasmalogens, vinyl ether bond is linking the sn-1 saturated or monounsaturated chain to the glycerol backbone of phospholipids (N. E. Braverman, et al., Biochimica et Biophysica Acta (BBA)-Molecular Basis of Disease 1822, 1442-1452 (2012)). It has been shown that ether lipids, PE and PC plasmalogens, can facilitate membrane fusion (P. E. Glaser, et al., Biochemistry 33, 5805-5812 (1994)) and increase membrane thickness of extracellular vesicles (X. Han, et al., Biochemistry 29, 4992-4996 (1990); T. Rog, et al., Biochimica et Biophysica Acta (BBA)-Biomembranes 1858, 97-103 (2016)), and therefore may play a role in nanovesicle uptake by cells. Detailed MS/MS analysis showed a high level of ether PE and PC species (plasmalogens) in both liquid-phase EV and MBV. These species were identified as PE-16:0p/20:4, PE-16:1p/20:4, PE-18:1p/20:4, PE-18:1p/22:6 and PC-16:0p/20:4, PC-18:0p/20:4, PC-20:0p/20:4, PC-18:0p/22:6, respectively (FIG. 4E).

TABLE 2
Contents of cardiolipin, phosphatidic acid, phosphatidylglycerol and
bis-monoglycerophosphate in MBV, exosomes and parent 3T3 cells.

| | 3T3 cells | Liquid-phase EV | MBV |
|---|---|---|---|
| Cardiolipin, CL | 0.97 ± 0.06 | 0.17 ± 0.01* | 0.66 ± 0.20* |
| Phosphatidic acid, PA | 0.11 ± 0.01 | 0.64 ± 0.29* | 0.42 ± 0.21 |
| Phosphatidylglycerol, PG | 0.04 ± 0.01 | 0.02 ± 0.02* | 0.11 ± 0.02** |
| bis-monoglycerophosphate, BMP | 0.13 ± 0.01 | 0.13 ± 0.04 | 0.55 ± 0.12#* |

Data are presented as pmol/nmol of phospholipids, means ± s.d.,
*p < 0.05 vs cells,
#p < 0.05 vs liquid-phase EV.

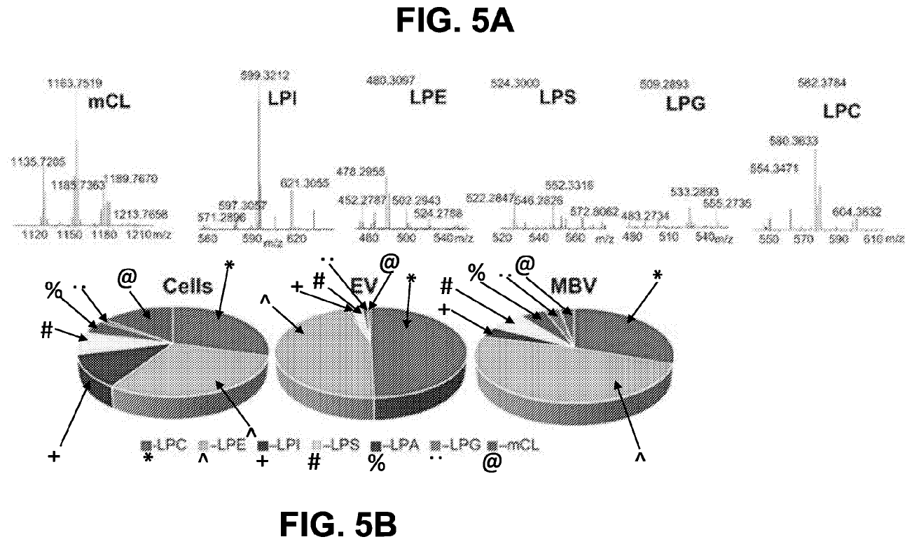

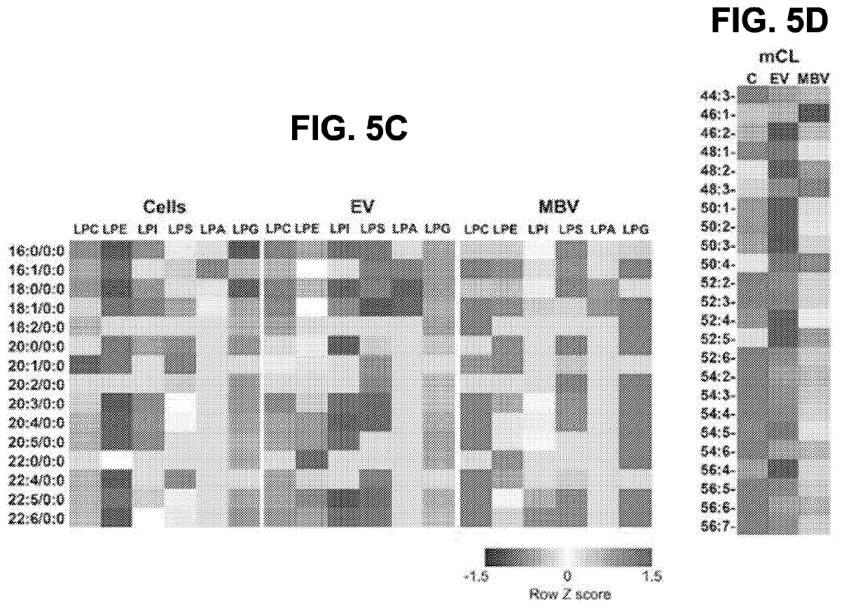

Lysophospholipid profiles of liquid-phase EV, MBV, and parent cells: Lysophospholipids (LPL), hydrolytic metabolites of phospholipids created by phospholipases A, are bioactive signaling molecules that modulate a variety of physiological responses, including macrophage activation (R. Ray, et al., Blood 129, 1177-1183 (2017)) inflammation and fibrosis (A. M. Tager et al., Nature medicine 14, 45 (2008)), tissue repair and remodeling (K. Masuda, et al., The FEBS journal 280, 6600-6612 (2013)), and wound healing (K. M. Hines et al., Analytical chemistry 85, 3651-3659 (2013)), among others. LC-MS analysis showed that LPL were present in all three types of samples albeit with their total content in MBV and liquid-phase EV being 1.7-1.8 times greater compared to the parent cells. More specifically, seven classes of LPL have been identified: lysophosphatidylethanolamine (LPE), lysophosphatidylcholine (LPC), lysophosphatidylserine (LPS) lysophosphoinositol (LPI), lysophosphatidic acid (LPA), lysophosphatidylglycerol (LPG) and monolysocardiolipin (mCL) (FIG. 5A). MBV were enriched in LPE, LPA and LPG compared to parent cells (FIG. 5B). The content of LPI and mCL was significantly lower in MBV and liquid-phase EV vs cells. The content of LPA and LPG was significantly higher in MBV compared to EV. The levels of mLCL and LPI in MBV were 3- and 6.3 times higher than in EV but 3.3- and 1.9 times lower compared to cells (FIG. 5C, FIG. 5D). No significant changes in the contents of LPE, LPC and LPS between MBV and EV were found. The non-oxidizable molecular species containing 16:0, 16:1, 18:0 and 18:1 were the major types found in all LPL species detected (FIG. 5C). These findings suggest that high levels of lysophospholipids, bioactive molecules that are important for macrophage differentiation, tissue repair, remodeling and wound healing, is a characteristic feature of MBV.

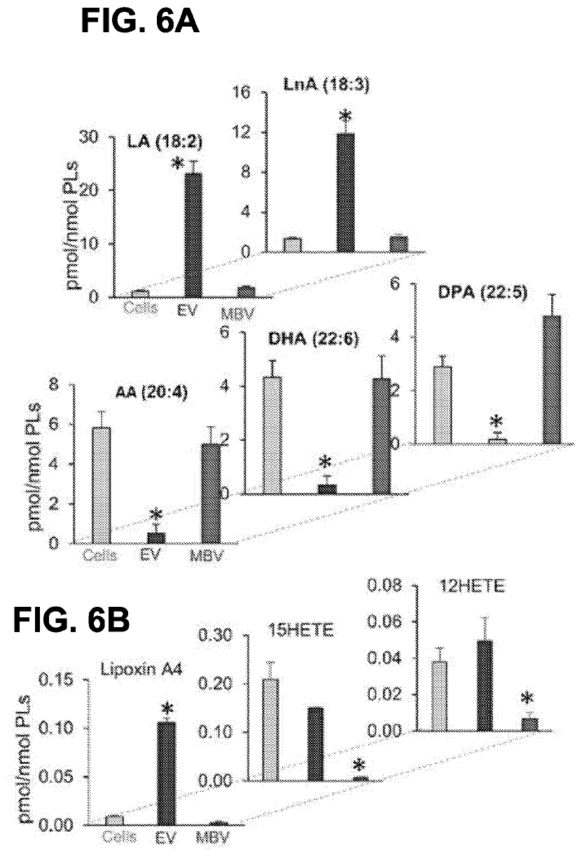

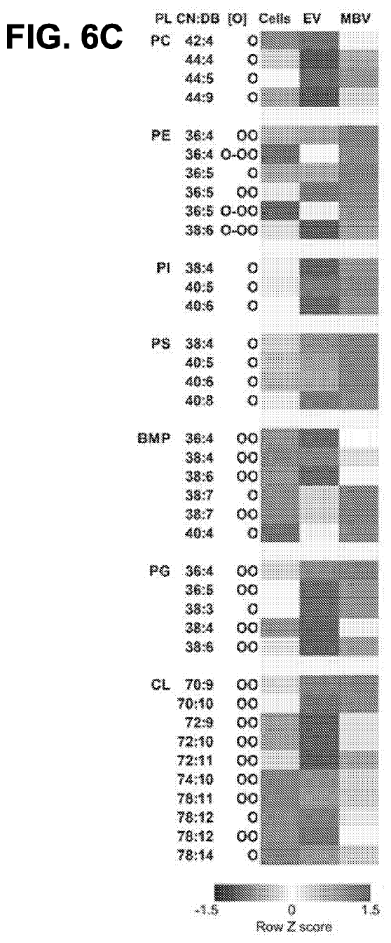

Analysis of free and oxygenated fatty acids of MBV and liquid-phase EV: As exposure of murine bone-marrow derived macrophages to MBV results in expression of M2-like markers, Fizz1 and ArgI, which are associated with a constructive macrophage phenotype (L. Huleihel et al., Science advances 2, e1600502 (2016)), LC/MS analysis of PUFA and their oxygenated products in MBV vs liquid phase EV and parent cells was performed. MBV were strongly enriched in arachidonic (20:4, AA), docosahexaenoic (22:6, DHA) and docosapentaenoic (22:5, DPA) fatty acids (FIG. 6A). In other words, MBV represent a reservoir of substrates for the biosynthesis of signaling lipid mediators by the respective enzymatic mechanisms—COXs and LOXs. In liquid phase EV, the major PUFA were linoleic (18:2) and linolenic (18:3) acids (FIG. 6A).

As extracellular vesicles contain enzymatic machinery for biosynthesis of AA-derived lipid mediators (E. Boilard, Journal of lipid research 59, 2037-2046 (2018)), redox lipidomics analysis of oxygenated fatty acids was performed. Higher levels of AA metabolites such as 12-HETE, 15-HETE, lipoxin A₄ were found in liquid-phase EV vs MBV (FIG. 6B). In the context of tissue repair, lipoxin A₄ (LXA₄) and D-series resolvin D1 (RvD1)—produced by 12/15-LOX from arachidonic (20:4, AA) and docosahexaenoic (22:6, DHA) acids—stimulate macrophage activation to the M2-like phenotype (C. N. Serhan, The American journal of pathology 177, 1576-1591 (2010)). Finally, oxidized phospholipids containing oxygenated AA and DHA in MBV and liquid-phase EV were characterized. The levels of oxygenated species were higher in MBV than liquid-phase EV, where PS, PI and PC were represented by mono-oxygenated species. BMP, PG and CL contained singly- and doubly-oxygenated AA- and DHA-residues; triply-oxygenated PUFA were found only in PE (FIG. 6C). Overall, lipidomics and oxidative lipidomics results show that the levels of free AA, DHA and DPA and PUFA-containing phospholipids as well as their oxidatively modified molecular species are higher in MBV compared to those in liquid-phase EV. MBV, but not liquid-phase EV, were enriched in PUFA non-oxygenated and oxygenated phospholipids and therefore represent a potential reservoir of oxidized and oxidizable esterified PL species, representing a potential source of lipid mediators activated by different phospholipases dependent on the pro-/anti-inflammatory contexts of the extracellular environment.

The above-mentioned LC-MS-based lipidomics and redox lipidomics studies were undertaken to conduct a detailed comparison of liquid-phase EV and MBV phospholipids. Combined with comprehensive RNA sequencing and bioinformatic analysis of the intravesicular cargo, these data reflect that MBV are a distinct and unique subpopulation of EV, distinct from liquid phase EV (i.e., exosomes), and a distinguishing feature of ECM-based biomaterials, with similarities limited to size and shape of the vesicles.

Herein, vesicle populations were fractionated based on their compartmentalization into either the liquid-phase cell culture medium or the solid-phase ECM substrate. In terms of composition, MBV isolated from the ECM of 3T3 fibroblasts contained a differential miRNA and lipid signature compared with liquid-phase EV and with the parent cell. These data are suggestive of a scenario in which molecular sorting occurs during vesicle biogenesis to specifically distribute miRNA and lipids to vesicles destined for different extracellular locations. Moreover, the cell's capacity to differentiate between a liquid interface and a solid substrate, and to selectively deposit tailored subpopulations of vesicles with distinct lipid signatures into these disparate compartments, provides evidence for a different and independent membrane biogenesis of MBV from the biogenesis of EV secreted into a liquid-phase. Considering that MBV were shown to be integrated within the dense fibrillar network of the extracellular matrix, MBV should be secreted by cells in concert with ECM components during matrix deposition during tissue development and homeostasis, and during dynamic matrix remodeling following injury. Furthermore, given that the ECM is a complex mixture of proteins, proteoglycans and glycosaminoglycans arranged in a tissue-specific 3D architecture (Hussey et al., Nature Review Materials, 3(7):159-173, 2018), MBV cargo and lipid content should also be unique to the tissue and cellular origin. MBV isolated from ECM bioscaffolds derived from anatomically distinct source tissue have differential miRNA signatures (Huleihel et al., Science Advances, 2, e1600502, 2016). Results from the present study further show that MBV isolated from ECM produced in vitro by bone marrow-derived stem cells, adipose stem cells and umbilical cord stem cells derived from different human donors contained a distinctive miRNA signature specific to the cell source. In addition, fewer miRNAs were found differentially expressed between BMSC and UCSC-derived MBV than between BMSC-ASC and UCSC-ASC, a finding that may be attributed to tissue-specific differentiation potentials of adipose stem cells (L. Xu et al., Stem cell research & therapy 8, 275 (2017)). These findings further underline the cell-specific features of MBV miRNA profiles, which was not significantly affected by the intrinsic variability of donors. However, given that the three human donors were all male, further studies to determine sex-related variations in the miRNA cargo of MBV from the stem cells samples are warranted. Importantly, principal component analysis showed a high degree of batch-to-batch consistency of the miRNA cargo from MBV deposited by specific cell types isolated from different human donors, supporting MBV and ECM biomaterial manufacturing as research tools or clinical therapeutics. The present study establishes that MBV integrated into the matrix are a unique subpopulation of EV. In addition, MBV showed a marked decrease in proteins commonly attributed to exosomes (e.g., CD63, CD81, CD9).

In contrast to EV that are secreted into body fluids and readily available for cell-cell communication, MBV embedded within tissue ECM are stably associated with the matrix and can only been isolated following degradation of the ECM material (Huleihel et al., Science advances 2, e1600502 (2016)). The requirement for matrix degradation to release MBV may partially define their mechanism of action, including those related to their capacity to generate pro-resolving lipid mediators. Because MBV remain intact and attached to ECM even after decellularization, the molecular speciation of their constituent phospholipids likely facilities such MBV-ECM interactions. Using LC-MS-based lipidomics and redox lipidomics approaches, detailed characterization of the molecular speciation of MBV phospholipids, lysophospholipids and the oxygenated and non-oxygenated PUFA were performed. High levels of lysophospholipids, bioactive molecules that are important for macrophage differentiation, tissue repair, remodeling and wound healing, is a characteristic feature of MBV. In addition, as fusogenic lipids, lysophospholipids can facilitate the transfer of the vesicular contents to intracellular targets. MBV, but not liquid-phase EV, were enriched in PUFA non-oxygenated and oxygenated phospholipids and therefore represent a potential reservoir of oxidized and oxidizable esterified PL species. Notably, PUFA-enriched MBV are an important source of lipid mediators activated by different phospholipases dependent on the pro-/anti-inflammatory context of the extracellular environment.

### Example 2: Use of MBV for Treatment of Pristane-Induced Arthritis

Urinary bladder matrix was prepared using the methodologies as described in Example 1.

MBV were isolated from laboratory produced porcine UBM by enzymatic digestion with Liberase TL (highly purified Collagenase I and Collagenase II) in buffer (50 mM Tris pH7.5, 5 mM CaCl₂), 150 mM NaCl) for 24 hours at room temperature on an orbital rocker. Digested ECM was then subjected to centrifugation at 10,000×g for 30 minutes to remove ECM debris. The clarified supernatant containing the liberated MBV was then centrifuged at 100,000×g (Beckman Coulter Optima L-90K Ultracentrifuge) at 4° C. for 2 hours to pellet the MBV.

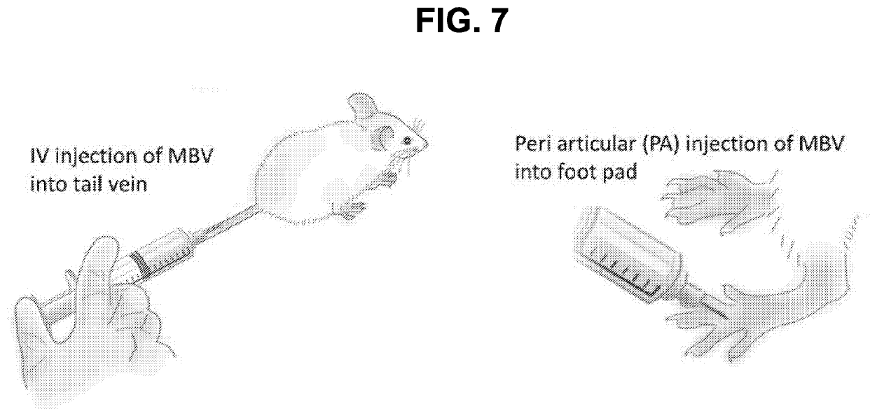

The Pristane-induced arthritis model in rats has been established as a clinically-relevant animal model for studying rheumatoid arthritis (Tuncel et al. PLoS One. 2016; 11(5):e0155936). Pristane induced arthritis was induced in 8-week-old, female, Sprague-Dawley rats by an intradermal injection of 300 μL Pristane (2,6,10,14-tetramethypentadecane) at the dorsal side of the tail, 1 cm distal to the base on Day 0 of the study. Negative control animals did not receive an intradermal injection of Pristane on Day 0. A second dose of 300 μL pristane was administered intradermally, approximately 1 cm distally to the dorsal tail base on Day 4. Animals receiving Pristane were housed together in cages. Animals receiving Pristane were randomized into the following experimental groups: Pristane-only+PBS, Pristane+i.p. Methotrexate (MTX), Pristane+periarticular (p.a.) MBV, and intravenous (i.v.) MBV. A depiction of periarticular and intravenous MBV routes of administration is shown in FIG. 7.

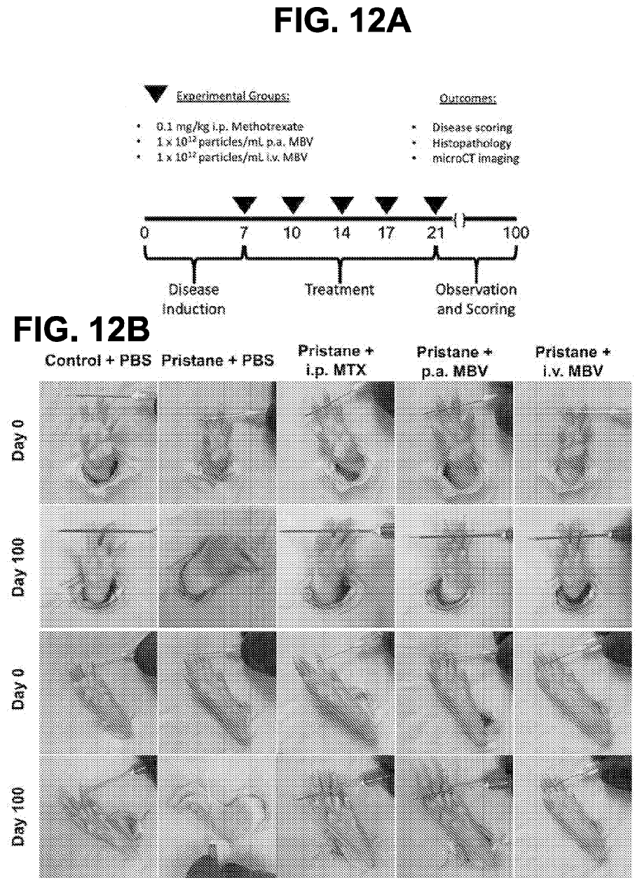

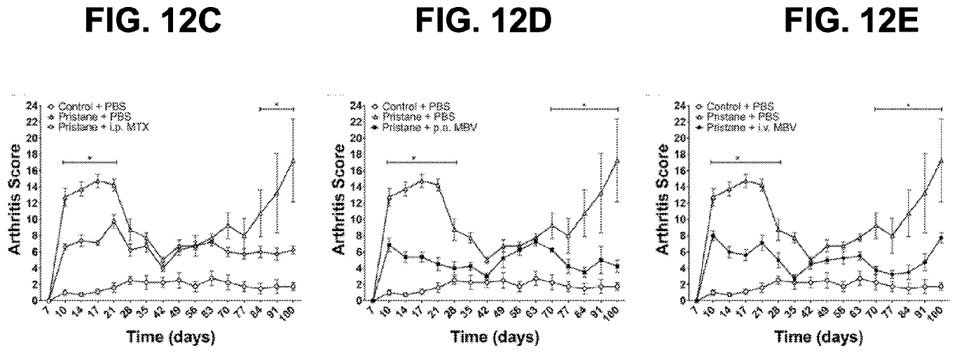

An arthritis score was determined on days 7, 10, 14, 17, 21, 28, and every week thereafter through an endpoint of 100 days for each animal. Photographs of each forepaw and hindpaw were taken as viewed from the volar and plantar perspectives, respectively. Qualitative arthritis severity was evaluated by two independent reviewers using a 60-point arthritis scoring criteria: 1 point was given for each inflamed knuckle or toe, and up to 5 points was assigned for an affected ankle (15 points per paw, 60 points per each rat). Animals designated as Pristane-only+PBS did not receive any treatment on days 7, 10, 14, 17, and 21. The Pristane+i.p. methotrexate animals received 0.1 mg/kg methotrexate in 1× sterile PBS (pH 7.4) delivered intra-peritoneally (i.p.) on days 7, 10, 14, 17, and 21. The Pristane+periarticular MBV animals received 25 μL of 500 μg/mL porcine-derived UBM MBV (1×10¹¹ particles/mL) delivered in the plantar and volar surfaces of hindpaws and forepaws, respectively. The intravenous MBV group received 100 μL of 500 μg/mL UBM MBV (1×10¹¹ particles/mL) delivered intravenously into the lateral tail vein of the animal. (FIG. 12A) Four animals in each group were assigned to a short-term study of 28 days and four animals were assigned to a 100 day study. Sample size was determined using previously published effect size of methotrexate with a predetermined alpha 0.05 and beta 0.80. Arthritis score was represented as mean+/−standard error of mean. Days 7-21 represent an n of 8 for each group and then days 28 and onward represent an n of 4 for each group. Differences in groups were analyzed using two-way analysis of variance with Tukey's post-hoc correction. Significance was determined prior to the study as p<0.05.

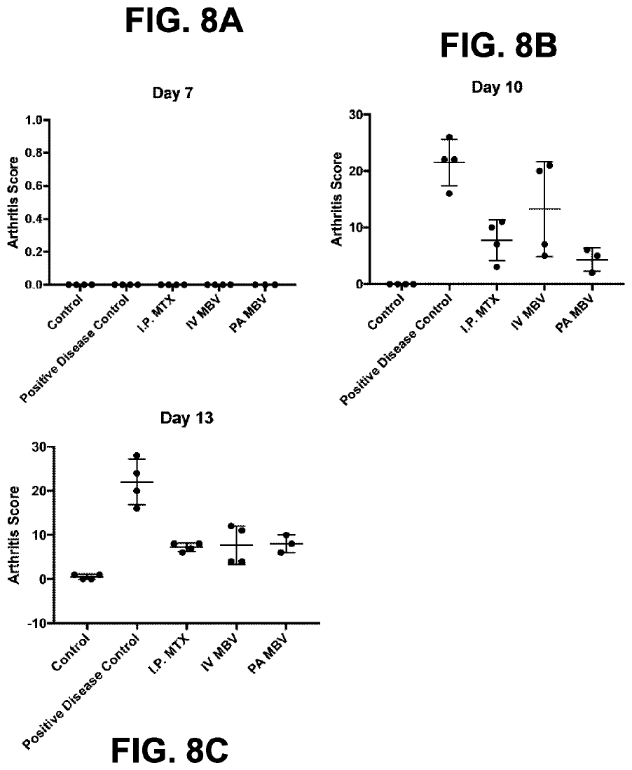

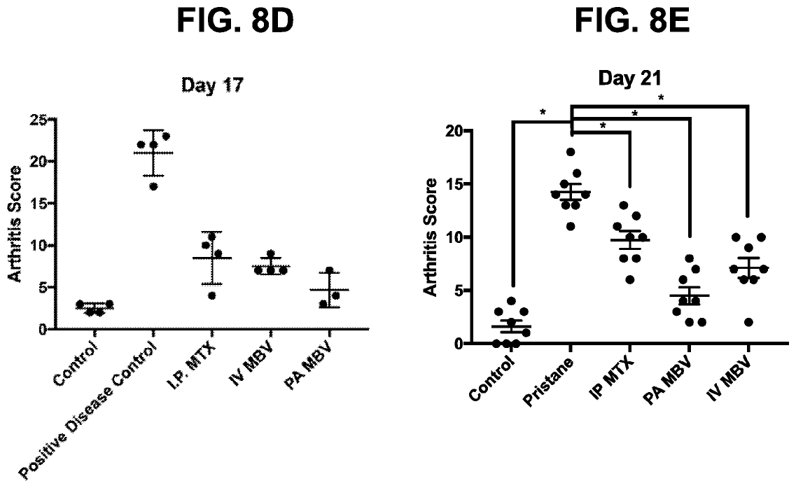

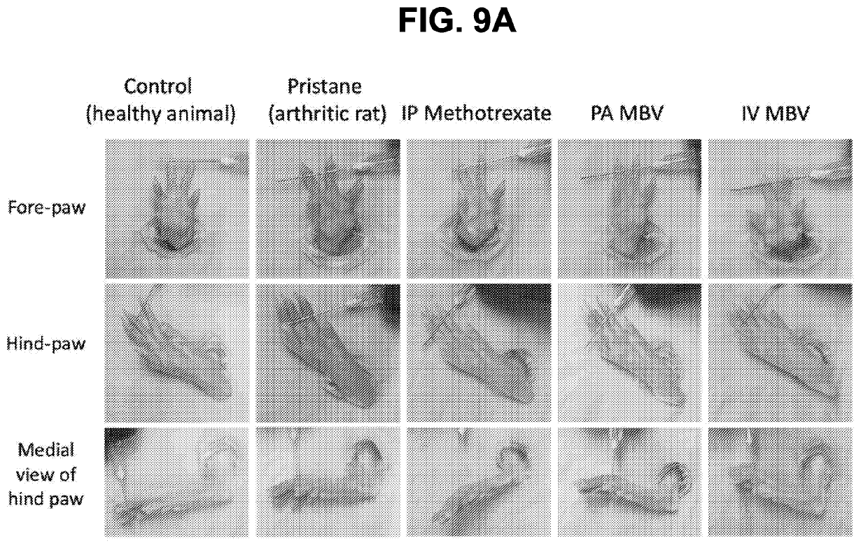

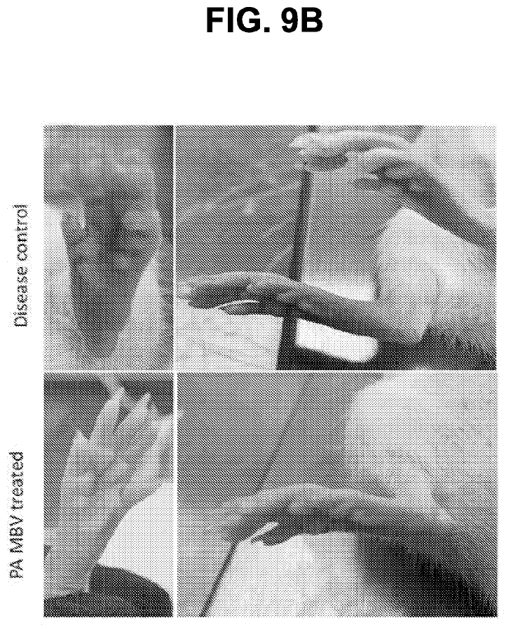

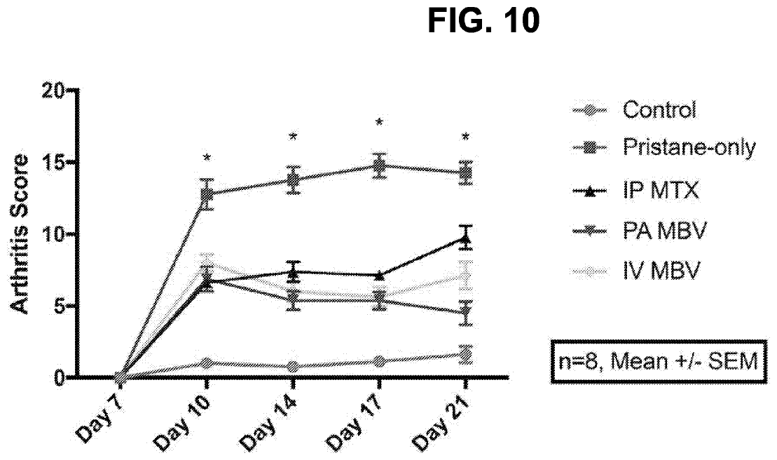

Administration of MBV, both through targeted PA (periarticular) and systemic IV administration, significantly reduced the severity of arthritis in the rats, displaying similar efficacy to the gold standard of care, methotrexate. While all rats displayed arthritis scores of 0 at Day 7 (FIG. 8A), as early as Day 10, high arthritis score was observed in Pristane-only rats, and all rats receiving treatment displayed lower arthritis scores (FIG. 8B). Surprisingly, beginning at Day 13, MBV treatment was as efficient as the gold-standard of arthritis treatment, methotrexate (FIG. 8C and FIG. 8D). By Day 21, MBV treated rats (both IV and PA treated) displayed lower arthritis scores than methotrexate treated rats (FIG. 8E). Photographs taken of the rat paws demonstrate differences in erythema and edema in Pristane-only and methotrexate and MBV treated rats (FIG. 9A-FIG. 9B). Average arthritis scores across treatment groups for the first 21 days of the experiment is shown in FIG. 10. While PA administration of MBV administration displayed a comparable reduction in arthritis score compared to Pristane-only rats, and a reduced arthritis score comparable to methotrexate treatment, it was unexpectedly discovered that intravenous administration had the same efficacy in reducing arthritis score as PA and methotrexate. While it was predicted that IV administration would result in a dilution of the potency of MBV and thus would have a limited, if any, effect on inflamed joints, this was not observed. Surprisingly, both PA and IV routes of MBV administration displayed comparable reductions in arthritis score compared to Pristane-only rats, and both reduced arthritis score comparably to methotrexate treatment. As peri-articular injection at a site of inflammation is painful and many joints in an individual may be inflamed, the unexpected finding that intravenous administration of MBV is just as effective as PA administration suggests that the systemic delivery route can be used for a less invasive yet equally effective therapeutic effect, requiring only a single injection per administration (rather than multiple injections per joint) and therefore being more comfortable for a patient.

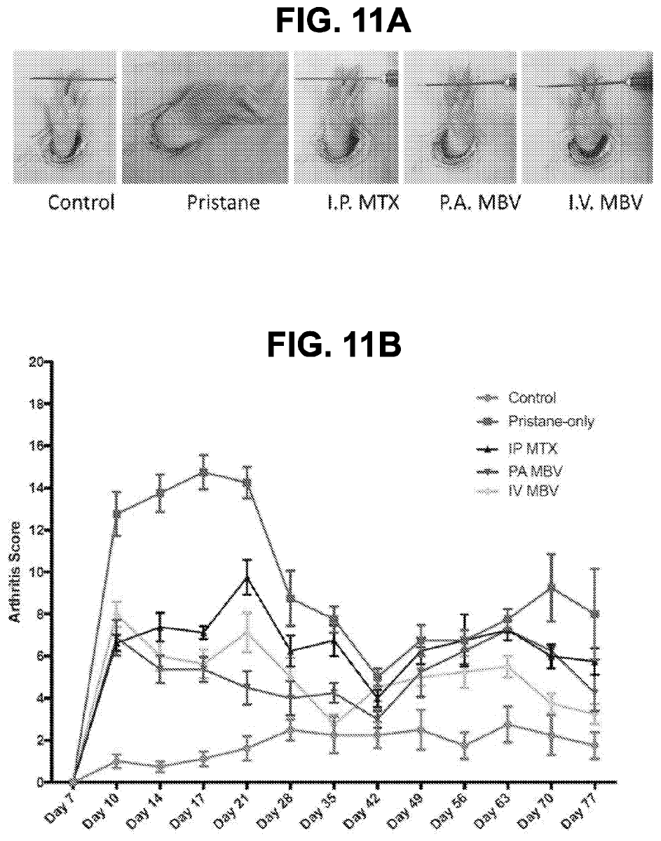

Unexpectedly, inflammatory recurrence was also decreased in rats receiving MBV both by IV and PA administration. Data collected through Day 77 of the experiment support MBV treatment as an efficient therapy for arthritis in chronic and relapsing phases of inflammation. As shown in the photographs in FIG. 11A, phenotypically, rat paws treated with PA or IV MBV display equivalent erythema and edema to those treated with methotrexate. PA and IV MBV reduce Pristane-induced arthritis clinical scoring in the chronic and relapsing phases of inflammation at equal efficacy to methotrexate, the gold standard of care for Rheumatoid Arthritis (FIG. 11B). Analysis of tissue from the rat model of rheumatoid arthritis resulted in tissue inflammation and decreased space between the tissue. In the MBV-treated samples, there was restoration of space and decreased inflammation comparable to that observed with methotrexate treatment, revealing the efficacy of MBV at both the organismal and tissue-levels.

In the acute phase of disease, designated as days 0-42, visual disease severity in the vehicle treated diseased animals (pristane+PBS) peaked at day 17, with a peak disease score of 14.8±0.8. Intra-peritoneal (i.p.) MTX treatment of diseased animals (pristane+i.p. MTX) reduced acute disease severity at days 10, 14, 17, and 21, with a peak disease score at day 21 (9.8±0.8) (FIG. 12C, p<0.05). The local, peri-articular (pristane+p.a. MBV) administration reduced disease severity at days 10, 14, 17, 21, and 28, with a peak disease score of 6.9±0.9 at day 10 (FIG. 12D, p<0.05). The systemic, intravenous (pristane+i.v. MBV) administration reduced disease severity at days 10, 14, 17, 21, and 28, with a peak disease score of 8.0±0.6 at day 10 (FIG. 12E, p<0.05). There were no significant differences among the three treatment groups in the acute phase (p>0.05); all three treatment groups were different than the disease-free control group (p>0.05). In summary, i.p. MTX, p.a. MBV, and i.v. MBV are equally effective in reducing pristane-induced RA disease severity in the acute phase of disease.

Since RA is a disease of a chronic relapsing-remitting phenotype, animals were observed after the acute phase to discern the long-term effect of MBV administration on chronic disease development. After day 28, through the completion of the study at day 100, no additional MTX or MBV treatments were administered. At the beginning of the chronic phase (day 42), disease severity had subsided and there was no difference among the following groups from days 42-63 (p>0.05): pristane+PBS, pristane+i.p. MTX, pristane+p.a. MBV, and pristane+i.v. MBV. At day 70, the pristane+PBS group began to develop a second disease flare-up that continued to rise until day 100, at which time the final disease severity score was 17.3±5.1. In contrast, the pristane+MTX, +p.a. MBV, and +i.v. MBV groups did not develop this upward trend through day 100. Further in contrast, the administration of MTX, p.a. MBV, and i.v. MBV resulted in a significant decrease in disease severity from days 84-100 for MTX (FIG. 12C, p<0.05), from days 70-100 for p.a. MBV (FIG. 12D, p<0.05), and from days 70-100 for i.v. MBV (FIG. 12E, p<0.05). While all three treatment conditions prevented a relapse in disease severity at day 100, there was no difference in disease scores among the three treatment groups (p>0.05). This data shows, surprisingly, that systemic administration is effective and non-toxic, and does not exhibit a dilution effect as was predicted.

The data suggest that MBV delivered locally and systemically prevent acute and chronic development of pristane-induced arthritis with comparable efficacy to methotrexate. Surprisingly, an initial treatment course of MBV, either systemically or locally, MBV can have a therapeutic effect in relieving arthritis symptoms for weeks to months after the initial treatment course of MBV ends, thereby reducing the severity or frequency of subsequent flares of rheumatoid arthritis symptoms or even eliminating them, resulting in remission. Further, it surprisingly was found that systemically administered MBV did not experience a dilution effect and were as effective as MBV locally administered periarticularly.

### Example 3: Matrix-Bound Nanovesicles Decrease Synovial Inflammatory Infiltration, Articular Cartilage Destruction, and Articular Proteoglycan Loss in Pristane-Induced Arthritis

Synovial inflammation, cartilage destruction, and proteoglycan loss are fundamental histopathologic changes that occur in RA disease progression. To determine the effect of MBV treatment on these histopathologic parameters, rat hind paws were collected at study termination for histopathologic imaging and analysis (day 100) from the animals from Example 2.

One hindpaw from each animal was used for histopathological analysis. Tissue specimens were fixed with 10% formalin in PBS, pH 7.4, decalcified using 5% formic acid, and embedded in paraffin wax. Sections were stained with hematoxylin and eosin (H&E) for examination by light microscopy for joint histology and pathology. Sections were stained with both a Toluidine Blue and an eosin counterstain for examination by light microscopy to assess proteoglycan composition of the articular cartilage.

Inflammation and joint damage of the tibiotalar joint were investigated by using an adapted three-parameter scoring system. Inflammation was scored on a scale of 0-3 (with 0 representing no inflammation and 3 a severe inflamed joint) depending on the relative proportion of inflammatory cells in the synovial tissue. Cartilage destruction was scored on a scale of 0-3, with results ranging from the appearance of dead chondrocytes and empty lacunae to complete loss of articular cartilage. Loss of proteoglycans in cartilage was scored on a scale of 0-3, and here results ranged from fully stained cartilage by toluidine blue staining to complete loss of articular cartilage. For each group a composite score was calculated for all parameters. Histology scores are represented as mean±standard error.

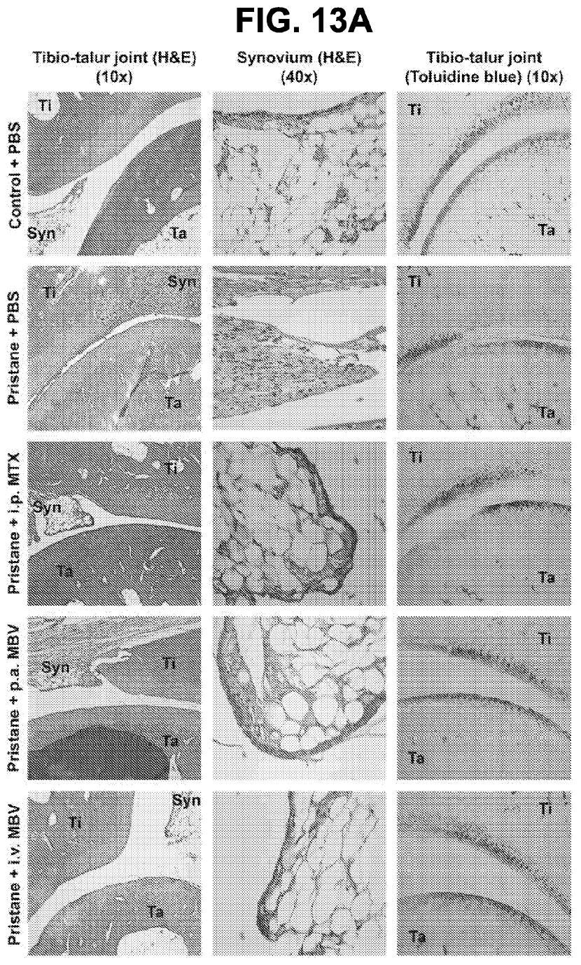

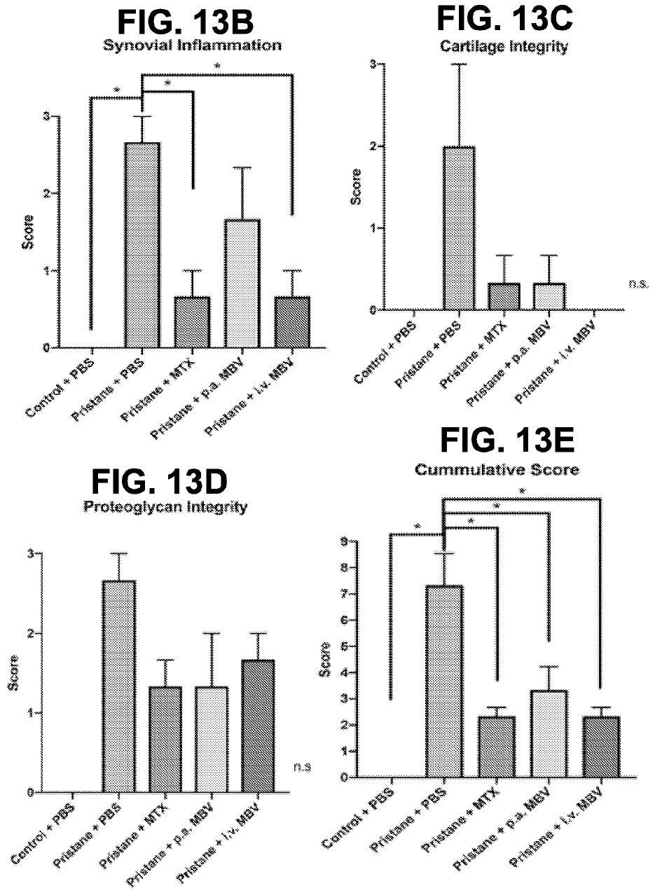

Vehicle-treated animals developed substantial joint pathology characterized by increased synovial inflammatory cell infiltration, articular cartilage degradation, and articular proteoglycan loss (FIG. 13A). Compared to the control+PBS group, the pristane+PBS group had an increase in synovial inflammation (2.7±0.3 vs 0.0±0.0), cartilage destruction (2.0±1.0 vs 0.0±0.0), and proteoglycan loss (2.7±0.3 vs 0.0±0.0) compared to the group of negative control animals (FIGS. 13A-13E, p<0.05). Compared to the pristane+PBS group (2.7±0.3), all treatment groups-Pristane+MTX (0.7±0.3), p.a. MBV (1.7±0.7), and i.v. MBV (0.7±0.3)—showed reduced synovial infiltration and inflammation by histologic scoring (FIGS. 13A-13E, p<0.05). While there was no significant difference between the pristane+PBS group and the three treatment groups regarding cartilage destruction and proteoglycan loss, all three treatment groups showed a reduction in these parameters (FIG. 13C and d., p>0.05). Compared to the pristane+PBS group, all treatment groups-again Pristane+MTX, p.a. MBV, and i.v. MBV-showed a reduction in the cumulative score of all three parameters (pristane+PBS (7.3±1.2) vs Pristane+MTX (2.3±0.3), and Pristane+i.v. MBV (2.3±0.3) (FIG. 2. e., p<0.05). No differences were observed among the three treatment groups across all three histologic parameters (FIGS. 12A-13E, p>0.05). This data shows that systemic administration is effective and non-toxic.

### Example 4: Matrix-Bound Nanovesicles Reduce Inflammatory Cell Infiltration and Promote Modulation of Pro-Inflammatory Synovial M1-Like Macrophages Toward Anti-Inflammatory M2-Like Macrophages

Using histologic sections from the previously mentioned samples, macrophage phenotype in the synovial tissue adjacent to the tibiotalar joint was assessed using immunohistochemistry. Paraffin-embedded tissue sections were deparaffinized using three progressive washes of xylene followed by rehydration using decreasing ethanol exchanges from 100% to 70% ethanol. Antigen retrieval was performed using commercially available DeCal solution per manufacturer's protocol (BioGenex). Sections were then blocked for one hour at room temperature using 5% bovine-serum albumin in 1× tris-buffered saline, pH 7.4. After block, sections were incubated ˜18 hours at 4° C. with the following primary antibodies and dilutions: goat anti-CD68 (1:100), rabbit anti-TNFalpha (1:100), and mouse anti-CD206 (1:100). Following primary antibody incubation, sections were incubated for 1 hour at room temperature with the following fluorescently-conjugated secondary antibodies: anti-rabbit ALEXAFLUOR® 300, anti-mouse ALEXAFLUOR® 488, and anti-goat ALEXAFLUOR® 594. Sections were counterstained with DRAQ5 nuclear stain and imaged.

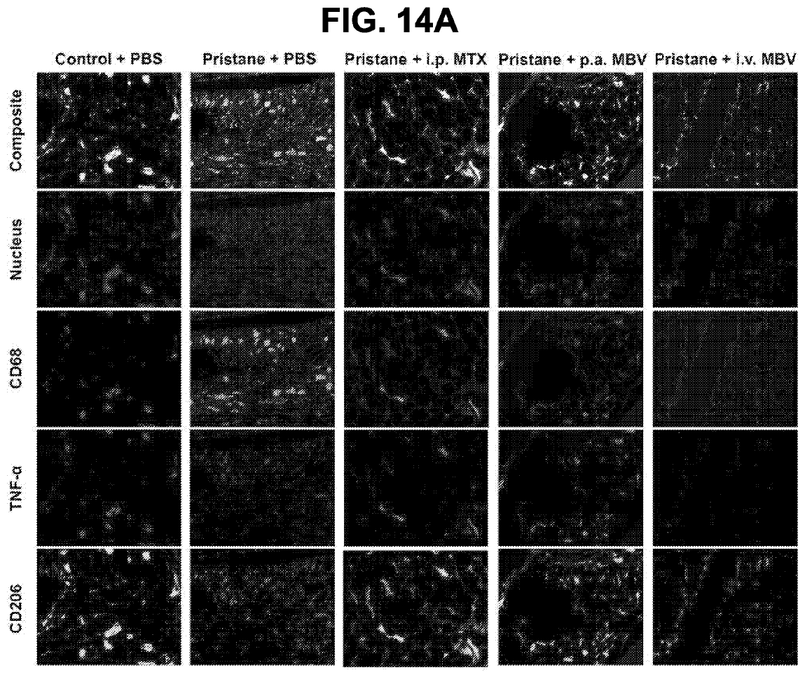

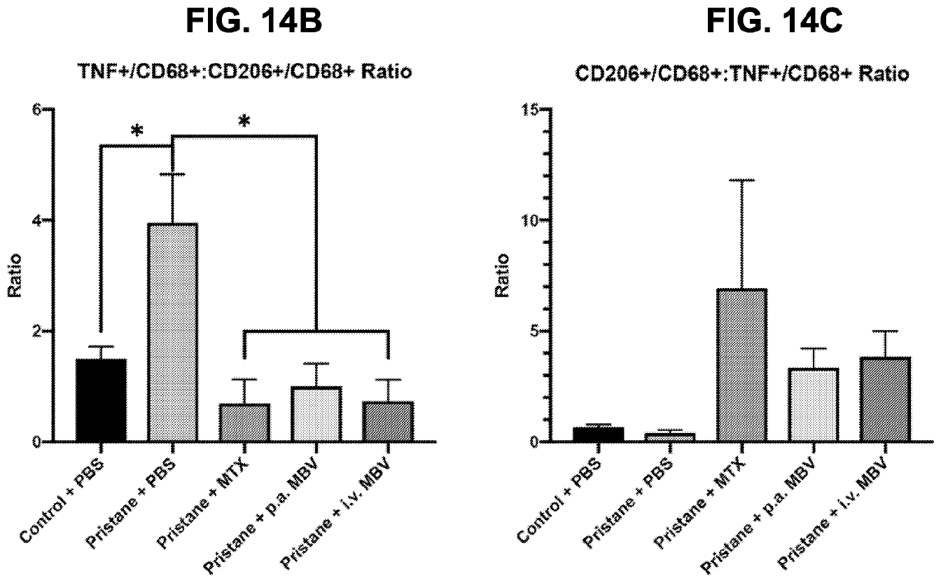

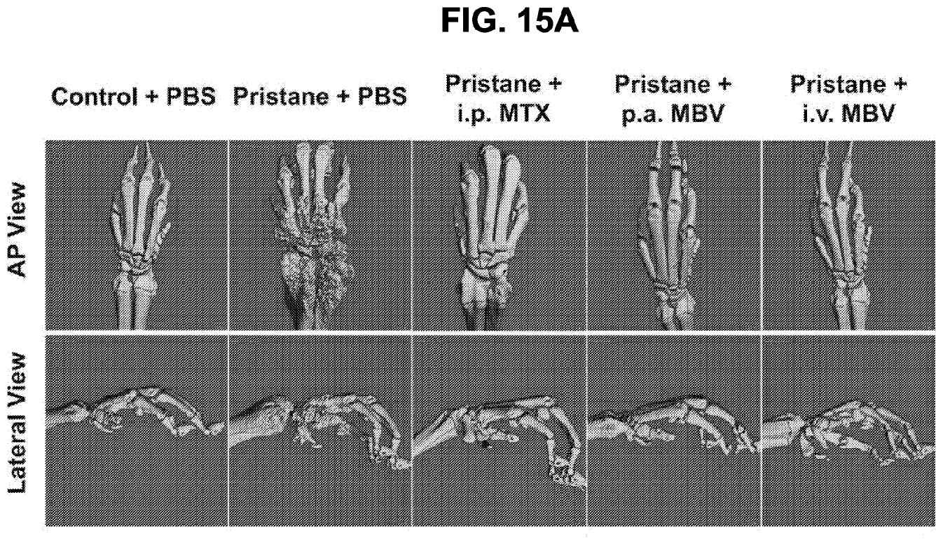

The disequilibrium between pro-inflammatory M1-like macrophages and M2-like macrophages is a key component of RA pathology. Compared to Control+PBS, Pristane+PBS increased the ratio of synovial TNF-alpha+/CD68+, M1-like macrophages relative to synovial CD206+/CD68+, M2 macrophages (3.9±0.9 vs 1.5±0.2, FIGS. 14A and 14B, p<0.05). The ratio of M1-like:M2-like macrophages was decreased compared to Pristane+PBS in the Pristane+MTX (0.7±0.4, FIGS. 14A and 14B, p<0.05), Pristane+p.a. MBV (1.0±0.4, FIGS. 14A and 14B, p<0.05), and Pristane+i.v. MBV (0.7±0.4, FIGS. 14A and 14B, p<0.05). There were no significant differences in the ratio of M1-like:M2-like macrophages among treatment conditions as well as no significant differences between the treatment conditions and the control+PBS group (FIGS. 14A and 14B, p>0.05). Regarding the ratio of M2-like:M1-like macrophages, there were no differences between the treatment groups, the Control+PBS group, and the Pristane+PBS group (FIGS. 14A and 14C). While not statistically significant, an increase in the ratio of M2-like:M1-like macrophages was observed in the synovium of animals in all three experimental treatment groups compared to Control+PBS and Pristane+PBS (FIGS. 14A and 14C).

### Example 5 Matrix-Bound Nanovesicles Prevent Adverse Bone Remodeling and Joint Destruction in the Pristane-Induced Rheumatoid Arthritis Model

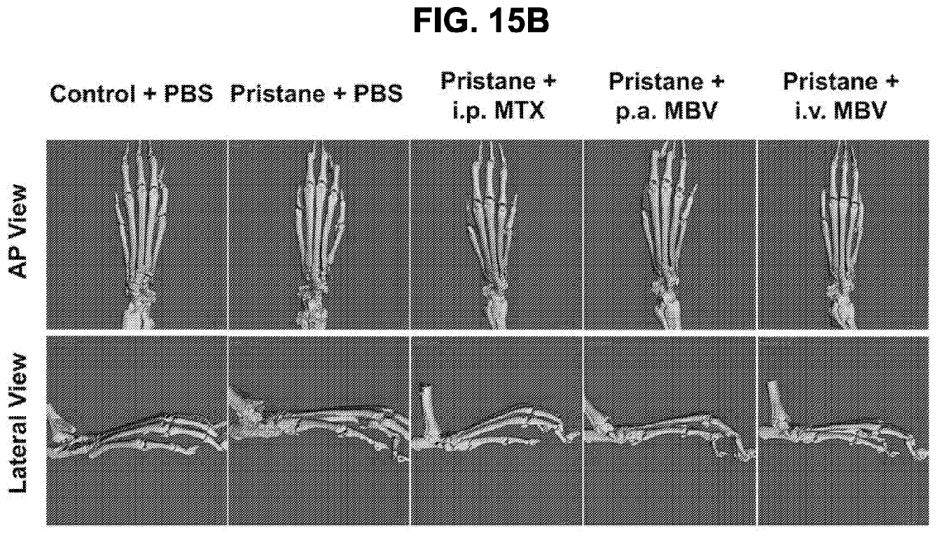

Micro-Computed Tomography (microCT) images were acquired of hindpaws after sacrifice at day 100 of the animals studied in Example 2. 3-D images were rendered using composite serial slice images and are shown in FIGS. 15A-B.

Compared to the pristane+PBS group, all three treatment groups substantially reduced qualitative bone damage and joint degeneration as observed by microCT imaging and 3-D reconstruction of the joints. In forepaws, i.p. MTX, p.a. MBV, and i.v. MBV administration substantially prevented adverse bone remodeling when compared to the vehicle control. These preventative changes occurred in both the fore- and hindpaws of animals. In the forepaw, damage was observed in articulation of the ulna and radius with the small carpal bones of the forepaw with minimal changes to the more distal interphalangeal joints and carpal-phalangeal joints (FIG. 15A). In the hindpaws, adverse remodeling was observed primarily at the articulation of the tibia and talus, as evident by fusion (syndesmosis) and absence of this joint on MicroCT (FIG. 15B). Again, this data shows that systemic administration of MBV significantly reduces qualitative bone and joint damage caused by RA. The effects were similar to periarticular administration of MBV, showing that surprisingly, there was no dilution effect in the systemic administration of MBV.

### Example 6: Use of Matrix Bound Vesicles (MBV) for Treatment of Collagen-Induced Arthritis

UBM and MBV derived from UBM are prepared using the methodologies as described in Example 2.

Collagen induced arthritis is induced in 8-week-old, female, Sprague-Dawley rats by a subcutaneous injection of 100 μL 200 μg/mL type II bovine collagen in emulsion with Freunds incomplete adjuvant at the dorsal side of the tail, 1 cm distal to the base on Day 0 of the study. Control animals do not receive an intradermal type II collagen emulsion on Day 0. A second dose of 100 μL 200 μg/mL type II collagen emulsion is administered subcutaneously, approximately 1 cm distally to the dorsal tail base on Day 7. Animals receiving type II collagen emulsion are housed together in cages. Animals receiving type II collagen emulsion are randomized into the following experimental groups: Type II collagen emulsion-only, Methotrexate, periarticular MBV, and intravenous MBV. Arthritis score is determined on days 7, 10, 14, 17, 21, 28, and every week thereafter through 100 days for each animal. Photographs of each forepaw and hindpaw are taken as viewed from the volar and plantar perspective, respectively. Arthritis is evaluated using a 60-point arthritis scoring criteria: 1 point is given for each inflamed knuckle or toe and up to 5 points are assigned for an affected ankle (15 points per paw, 60 points per each rat). Animals designated as Type II collagen emulsion-only do not receive any treatment on days 7, 10, 14, 17, and 21. The methotrexate animals receive 0.1 mg/kg methotrexate in 1× sterile PBS delivered intra-peritoneally on days 7, 10, 14, 17, and 21. The periarticular MBV animals receive 25 μL of 500 μg/mL porcine-derived, UBM MBV delivered in the plantar and volar surfaces of hindpaws and forepaws, respectively. The intravenous MBV group receive 100 μL of 500 μg/mL UBM MBV delivered intravenously into the lateral tail vein of the animal. Four animals in each group are assigned to a short-term study of 28 days and four animals are assigned to a 100-day study. Sample size is determined using previously published effect size of methotrexate with a predetermined alpha 0.05 and beta 0.80. Arthritis score is represented as mean+/−standard error of mean. Differences in groups are analyzed using two-way analysis of variance with Tukey's post-hoc correction. Significance was determined prior to the study with an alpha of 0.05.

The results show that animals treated with MBV demonstrate significantly decreased arthritis scores compared to untreated control animals, and the arthritis scores of MBV-treated animals is comparable to the arthritis scores of methotrexate-treated animals. Further, intravenous MBV treatment is found to be as efficacious as periarticular MBV treatment. These data demonstrate that MBV treatment for arthritis is equally efficacious to the gold standard of treatment for arthritis: methotrexate. Further, the data demonstrate that systemic administration of MBV has similar therapeutic efficacy as local administration in treating rheumatoid arthritis and that an initial administration of MBV, either systemically or locally, can have a therapeutic effect in relieving arthritis symptoms for weeks to months after the initial administration of MBV, thereby reducing the severity or frequency of subsequent flares of rheumatoid arthritis symptoms or even eliminating them.

### Example 7: Use of Matrix Bound Vesicles (MBV) for Treatment of Psoriasis

Preparation of urinary bladder matrix (UBM): UBM was prepared as previously described (Mase V J, et al. Orthopedics. 2010; 33(7):511). Porcine urinary bladders from market-weight animals were acquired from Tissue Source, LLC. Briefly, the tunica serosa, tunica muscularis externa, tunica submucosa, and tunica muscularis mucosa were mechanically removed. The luminal urothelial cells of the tunica mucosa were dissociated from the basement membrane by washing with deionized water. The remaining tissue consisted of basement membrane and subjacent lamina propria of the tunica mucosa and was decellularized by agitation in 0.1% peracetic acid with 4% ethanol for 2 hours at 300 rpm. The tissue was then extensively rinsed with PBS and sterile water. The UBM was then lyophilized and milled into particulate using a Wiley Mill with a #60 mesh screen.

Isolation of Matrix Bound Nanovesicles: MBV were isolated from laboratory produced porcine urinary bladder matrix (UBM) by enzymatic digestion with Liberase TL (highly purified Collagenase I and Collagenase II) in buffer (50 mM Tris pH7.5, 5 mM CaCl₂), 150 mM NaCl) for 24 h at room temperature on an orbital rocker. Digested ECM was then subjected to centrifugation at 10,000×g (30 min) to remove ECM debris. The clarified supernatant containing the liberated MBV was then centrifuged at 100,000×g (Beckman Coulter Optima L-90K Ultracentrifuge) at 4° C. for 2 hr to pellet the MBV.

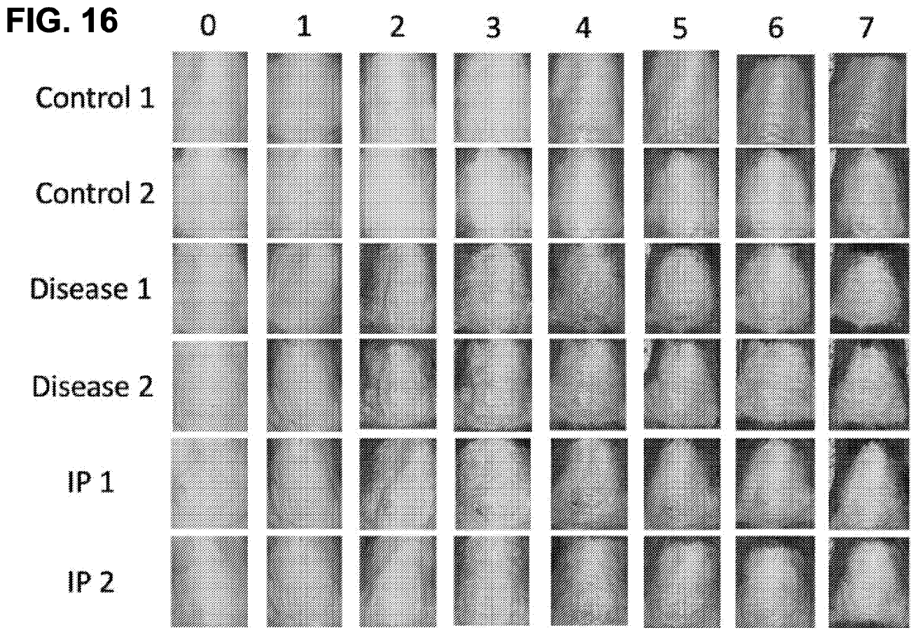

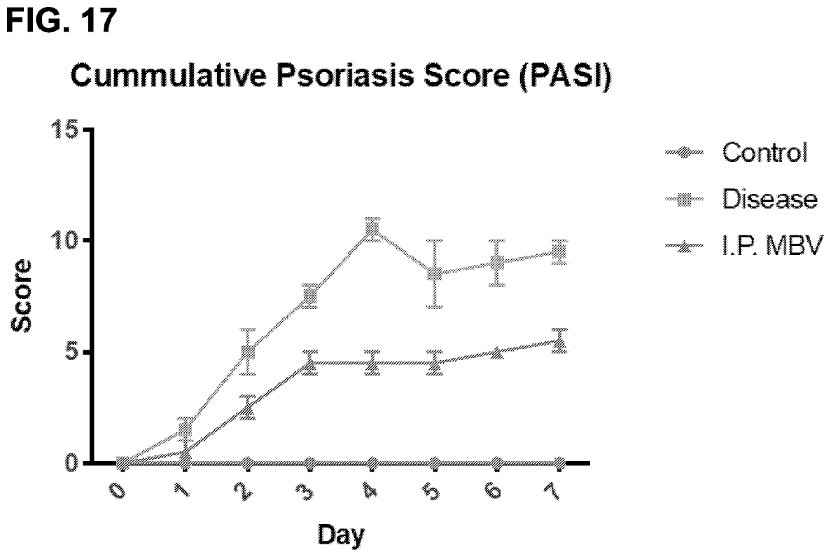

Induction of Psoriasis and Treatment Regimen: Psoriasis was induced in 8-week-old, female, C57/b16 mice by daily, topical application of 62.5 mg of 5% imiquimod cream to the shaved back and right pinna of mice for 7 days. Images of the shaved back and right pinna in the animals over 7 days is shown in FIG. 16. Control animals did not receive topical imiquimod throughout the study and instead received topical administration of petroleum jelly to a shaved back and right pinna. Treatment groups and therapeutic paradigms were divided down into prevention of psoriasis flares and management of existing flares. Those animals receiving preventative therapy received treatment between days 0-16 of the study and those animals receiving management therapy received treatment between days 7-16 of the study. Animals receiving intravenous MBV received 500 μg/mL porcine-derived, UBM MBV on each day of the study as designated by treatment timeline. Starting on day 0 through day 7, erythema, scaling, and thickness were scored by two independent reviewers using an objective scoring system modified from the clinical Psoriasis Area and Severity Index (PASI). A score of 0-4 was assigned for erythema and scaling with a score of 0=none, 1=slight, 2=moderate, 3=marked, 4=very marked. On each day, the thickness of the right ear pinna was measured using a micrometer and the thickness of the skin was scored based on the increase in the thickness compared with day—(1 for 20-40%, 2 for 40-60%, 3 for 60-80%, and 4 for >80%). The total score from each index was summed with a total scale for psoriatic inflammation represented on a 12-point total scale (0-12). Cumulative Psoriasis Scores (PASI) for the animals from days 0 through 7 are shown in FIG. 17.

Statistical analysis: Sample size was determined using previously published effect size of acitretin with a predetermined alpha 0.05 and beta 0.80. Psoriasis Area and Severity Index (PASI) score was represented as mean+/−standard error of mean. Differences in groups were analyzed using two-way analysis of variance with Tukey's post-hoc correction. Significance was determined prior to the study with an alpha of 0.05.

As demonstrated by the PASI scores and images of the animals provided, systemic administration of MBV reduces erythema and scaling in imiquimod-induced psoriasis. Additionally, systemic administration of MBV reduces skin thickness in imiquimod-induced psoriasis. PASI scores for MBV treated animals were significantly lower than untreated animals showing that MBV by systemic administration is a viable therapy for treating psoriasis.

### Example 8: MBV Do Not Produce an Immunosuppresive Effect

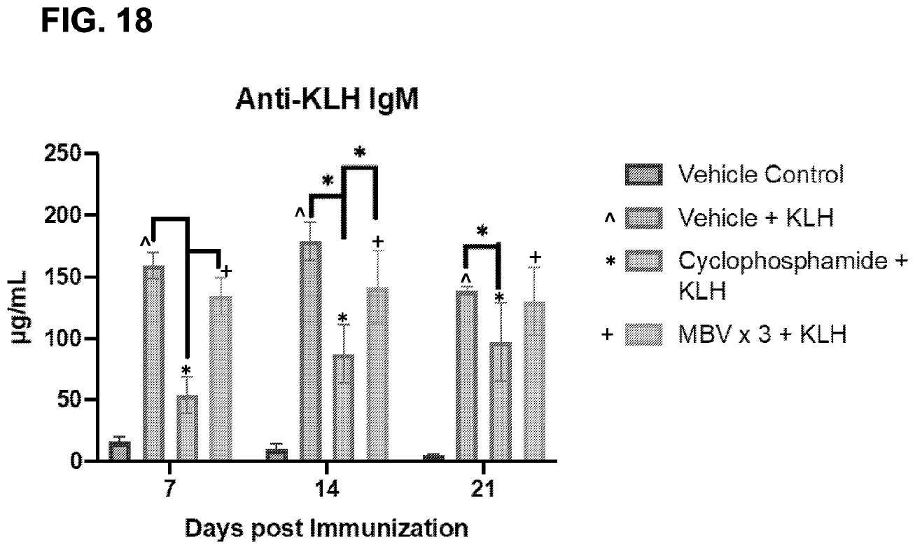

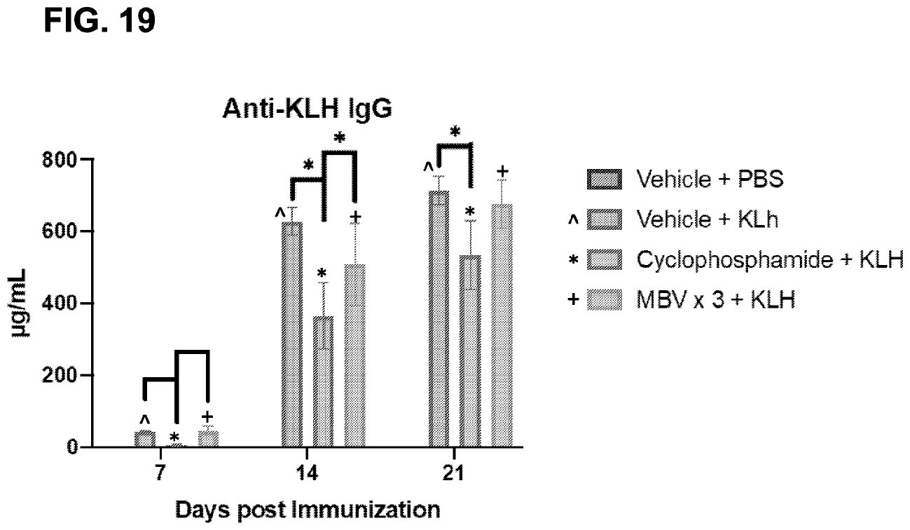

The Keyhold Limpet Hemocyanin (KLH) rat model of immunosuppression and immunotoxicity was used to assess immunotoxicity of MBV delivered systemically. 8-week-old Sprague-Dawley rats were separated into four separate groups: KLH control used to demonstrate normal anti-KLH response after immunization with KLH, Vehicle control used to control for any potential effects not related to treatment or KLH immunization, Cyclophosphamide positive control as a potent immunosuppressive, and an MBV treated group to assess the effect of MBV administration on systemic immunity. On Day 0, the cyclophosphamide treated animals received 200 mg/kg i.p. and the MBV treated animals received 1 mg/ml UBM MBV delivered intravenously on Day 0, 3, and 6. On Day 7, all groups besides the vehicle control were immunized with 0.4 mL of 1000 μg/ml reconstituted KLH in Freund's incomplete adjuvant i.p. On days 14, 21, and 28 (7, 14, and 21 days post-immunization), whole blood was collected from the lateral tail vein and serum was isolated for analysis of anti-KLH IgM and IgG. Anti-KLH IgM and IgG were assessed in the serum of all animals using ELISA. The results are shown in FIG. 18 (Anti-KLH IgM levels) and FIG. 19 (Anti-KLH IgG levels).

As shown in FIGS. 18 and 19, systemic administration of MBV before immunization with KLH does not affect the ability of a host animal to mount a normal IgG or IgM antibody response to the KLH antigen. Cyclophosphamide, a known immunosuppressant significantly reduces anti-KLH IgG and IgM levels at days 7, 14, and 21 compared to the Vehicle+KLH control. There is no significant difference between the Vehicle+KLH control and the MBV treated animals in the level of IgG or IgM produced.

These results demonstrate that systemic administration of MBV does not suppress a physiologic antibody immune response, and that in contrast to other standard treatments for autoimmune diseases that are immunosuppressive (like methotrexate and cyclophosphamide), MBV can be used to treat RA and other auto-immune diseases without suppressing the immune system. The side effects of immunosuppressive therapy, such as infection and cancer, can therefore be avoided by use of MBV to treat autoimmune diseases.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims.

1. A method of treating an autoimmune disorder in a subject in need thereof comprising administering to the subject via systemic administration a pharmaceutical preparation comprising a therapeutically effective amount of isolated matrix bound vesicles (MBV) derived from extracellular matrix, thereby treating the autoimmune disorder.

2. The method of claim 1, wherein the autoimmune disorder is a non-ocular autoimmune disorder.

3. The method of claim 1, wherein the autoimmune disorder is not rheumatoid arthritis, scleroderma, or ulcerative colitis.

4. The method of claim 1, wherein the autoimmune disorder is selected from Addison's disease, alopecia areata, ankylosing spondylitis, anti-phospholipid antibody syndrome, autoimmune hepatitis, Celiac disease, Crohn's disease, Goodpasture's Syndrome, Grave's disease, Guillain-Barre syndrome, Hashimoto's thyroiditis, immune thrombocytopenia, IgA Nephropathy, inflammatory bowel disease (IBD), multiple sclerosis, myasthenia gravis, pemphigoid, pemphigus, polyglandular autoimmune syndrome type 2, psoriasis, psoriatic arthritis, rheumatoid arthritis, scleroderma, Sjogren's syndrome, systemic lupus erythematosus, Takayasu's arteriosis, type 1 diabetes, ulcerative colitis, autoimmune encephalitis, or undifferentiated connective tissue disease (UCTD).

5.-16. (canceled)

17. The method of claim 1, wherein the subject experiences a therapeutic benefit for a prolonged period of time measured from the beginning of administration of the MBV or measured from the end of administration of the MBV.

18. The method of claim 17, wherein the subject experiences a therapeutic benefit beginning from the administration of MBV lasting a time period of at least 2 weeks, at least 3 weeks, at least 4 weeks, at least 1 month, at least 2 months, or at least 3 months.

19. The method of claim 17, wherein the subject experiences a therapeutic benefit measured from the end of administration of MBV lasting a time period of at least 2 weeks, at least 3 weeks, at least 4 weeks, at least 1 month, at least 2 months, or at least 3 months.

20.-23. (canceled)

24. The method of claim 1, wherein the systemic administration is selected from intravenous administration, oral administration, enteral administration, parenteral administration, intranasal administration, rectal administration, sublingual administration, buccal administration, sublabial administration, intraperitoneal administration, subcutaneous, or intramuscular administration.

25. (canceled)

26. The method of claim 1, wherein the MBV are administered in amount of 1×10⁶ to 1×10¹² MBV per kg of body weight per administration.

27. (canceled)

28. The method of claim 1, wherein the MBV are administered 1 time per week for 4 weeks, 1 time per week for 3 weeks, 1 time per week for 2 weeks, 1 time per week for 1 week, 2 times per week for 4 weeks, 2 times per week for 3 weeks, 2 times per week for 2 weeks, 2 times per week for 1 week, 3 times per week for 4 weeks, 3 times per week for 3 weeks, 3 times per week for two weeks, 3 times per week for 1 week, 4 times per week for 1 week, 4 times per week for two weeks, four times per week for three weeks, or 4 times per week for four weeks.

29. A method of treating psoriasis in a subject in need thereof comprising administering to the subject a pharmaceutical preparation comprising a therapeutically effective amount of isolated MBV derived from extracellular matrix, thereby treating the psoriasis in the subject.

30. The method of claim 29, comprising systemically administering the pharmaceutical preparation to the subject.

31. The method of claim 29, comprising locally administering the pharmaceutical preparation to the subject.

32. The method of claim 1, wherein a therapeutic benefit for the subject is a reduction in a symptom of the disorder present prior to administration of the MBV.

33. The method of claim 32, wherein a therapeutic benefit for the subject is a reduction in the level of inflammation in the subject over the level of inflammation prior to administration of the MBV.

34. The method of claim 32, wherein a therapeutic benefit is a remission of the disorder.

35. The method of claim 32, wherein the therapeutic benefit is a reduction in flares of the disorder's symptoms or elimination of flares of the disorder's symptoms during the time period.

36. The method of claim 1, wherein:
(i) the MBV do not express one or more of CD63, CD81, and/or CD9, or have barely detectable levels of CD63, CD81, and/or CD9; and/or
(ii) the MBV comprise:
(a) a phospholipid content comprising at least 55% phosphatidylcholine (PC) and phosphatidyl inositol (PI) in combination;
(b) a phospholipid content comprising 10% or less sphingomyelin (SM);
(c) a phospholipid content comprising 20% or less phosphatidylethanolamine (PE); and/or
(d) a phospholipid content comprising 15% or greater phosphatidylinositol (PI).

37. The method of claim 1, wherein the MBV are derived from extracellular matrix of urinary bladder, small intestine, heart, dermis, liver, kidney, uterus, brain, blood vessel, lung, bone, muscle, pancreas, stomach, spleen, colon, adipose tissue, or esophagus.

38. The method of claim 1, wherein the MBV are derived from urinary bladder matrix (UBM), small intestinal submucosa (SIS), or urinary bladder submucosa (UBS).

39. The method of claim 1, wherein the MBV are derived from extracellular matrix from a mammalian vertebrate selected from a human, monkey, pig, cow, or sheep.

40-46. (canceled)


SEQUENCE LISTING (3 sequences)

SEQ ID NO: 1 - RNA, 88 nt, Homo sapiens
caccuuguccucacgguccaguuuucccaggaaucccuuagaugcuaagauggggauuccuggaaauacuguucuugaggucaugguu

SEQ ID NO: 2 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 3 - DNA, 106 bp, Artificial Sequence
  DNA encoding miR-143
gcgcagcgccctgtctcccagcctgaggtgcagtgctgcatctctggtcagttgggagtctgagatgaagcactgtagctcaggaagagagaagttgttctgcagc